(19) United States
(12) Patent Application Publication
(10) Pub. No.: US 2024/0285580 A1
(43) Pub. Date: Aug. 29, 2024

(54) COMPOSITIONS AND METHODS FOR EXTENDED RELEASE CROMAKALIM THERAPY

(71) Applicants: Qlaris Bio, Inc., Dedham, MA (US); Mayo Foundation for Medical Education and Research, Rochester, MN (US)

(72) Inventors: Thurein M. Htoo, Westwood, MA (US); Barbara M. Wirostko, Park City, UT (US); Michael P. Fautsch, Rochester, MN (US)

(73) Assignees: Qlaris Bio, Inc., Dedham, MA (US); Mayo Foundation for Medical Education and Research, Rochester, MN (US)

(21) Appl. No.: 18/439,552

(22) Filed: Feb. 12, 2024

Related U.S. Application Data
(63) Continuation of application No. PCT/US2022/040197, filed on Aug. 12, 2022.
(60) Provisional application No. 63/232,603, filed on Aug. 12, 2021.

(51) Int. Cl. A61K 31/4025; A61K 47/50

(52) U.S. Cl. CPC A61K 31/4025; A61K 47/50

(57) ABSTRACT

Extended-release pharmaceutical compositions comprising a biodegradable microparticle, nanoparticle or other polymeric formulation of Formula I, Formula IL, or Formula III or a pharmaceutically acceptable salt thereof are provided with advantageous properties for in vivo delivery to a patient in need thereof. The extended-release formulations overcome historic obstacles in cromakalim, including levcromakalim, delivery.

### CROSS-REFERENCE TO RELATED APPLICATIONS

This application is a continuation of International Patent Application No. PCT/US2022/040197, filed in the U.S. Receiving Office on Aug. 12, 2022, which claims the benefit of U.S. Provisional Application No. 63/232,603, filed Aug. 12, 2021. The entirety of each of these applications is incorporated by reference for all purposes.

### FIELD OF THE INVENTION

This application is in the field of medical therapy and provides new pharmaceutical compositions and methods of use comprising certain extended-release formulations of cromakalim, including levcromakalim, and their pharmaceutically acceptable salts.

### STATEMENT REGARDING FEDERALLY SPONSORED RESEARCH OR DEVELOPMENT

This invention was made with government support under Grant No. R01 EY21727 awarded by the National Institutes of Health. The government has certain rights in the invention.

### BACKGROUND OF THE INVENTION

Cromakalim and its use as an anti-hypertensive was first described in European Patent EP 0120428B1 assigned to the Beecham Group, Inc. Disclosures of cromakalim's effects on intraocular pressure and glaucoma were reported in PCT Application WO 89/10757; Lin et al., “Effects of Cromakalim and Nicorandil on Intraocular Pressure after Topical Administration in Rabbit Eyes” Journal of Ocular Pharmacology and Therapeutics, 1995, 11, 195; and, Roy Chowdhury et al., “Ocular Hypotensive Effects of the ATP-Sensitive Potassium Channel Opener Cromakalim in Human and Murine Experimental Model Systems” PLOS One, 2015, 10, e0141783.

Cromakalim and diazoxide were reported to lower blood pressure in Quast, U. et al. J Pharmacol Exp Ther 1989, 250, 261. Additionally, publications by Chowdhury et al. and Roy Chowdhury et al. describe the use of diazoxide and nicorandil (“ATP-Sensitive Potassium (KATP) Channel Openers Diazoxide and Nicorandil Lower Intraocular Pressure” IOVS, 2013, 54, 4894 and “ATP-Sensitive Potassium (KATP) Channel Activation Decreases Intraocular Pressure in the Anterior Chamber of the Eye” IOVS, 2011, 52, 6435). Cromakalim placed in membrane patches from rabbit mesenteric arterial smooth muscle cells increases the open-state probability (Pₒₚₑₙ) of single K_{ATP} channels more than 9-fold in the presence of ATP (Brayden, J. E. et al., Blood Vessels, 1991, 28, 147). Other ATP-sensitive potassium channel openers include pinacidil and minoxidil sulfate, which act as vasodilators in vitro and in vivo.

Cromakalim exists as a mixture of diastereomers in the trans-configuration (a mixture of (3R,4S) and (3S,4R) diastereomers):

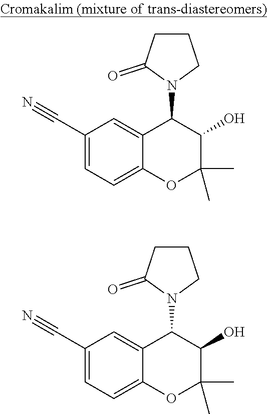 [SMILES: CC1(C)Oc2ccc(C#N)cc2[C@@H](N2CCCC2=O)[C@@H]1O ; CC1(C)Oc2ccc(C#N)cc2[C@H](N2CCCC2=O)[C@H]1O]

The (3S,4R)-diastereomer is also referred to as (−)-cromakalim or levcromakalim and the (3R,4S)-diastereomer is also referred to as (+)-cromakalim or dexcromakalim:

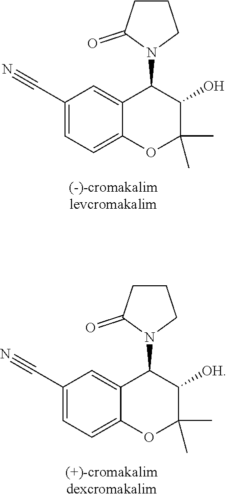 [SMILES: CC1(C)Oc2ccc(C#N)cc2[C@@H](N2CCCC2=O)[C@@H]1O ; CC1(C)Oc2ccc(C#N)cc2[C@@H](N2CCCC2=O)[C@@H]1O]

The majority of cromakalim's reported activity stems from the (3S,4R)-diastereomer, levcromakalim (Ashwood et al. Synthesis and Antihypertensive Activity of 4-(Cyclic Amido)-2H-1-benzopyrans” J. Med. Chem. 1986, 29, 2194 and Attwood et al. “Synthesis of Homochiral Potassium Channel Openers: Role of the Benzopyranyl 3-Hydroxyl Group in Cromakalim and Pyridine N-Oxides in Determining the Biological Activities of Enantiomers” Bioorg. Med. Chem. Lett. 1992, 2, 229).

Additional publications describing the use of cromakalim and levcromakalim prodrugs for the treatment of medical disorders include WO2021/158992 and WO2021/119503 assigned to Qlaris Bio, Inc. and Mayo Foundation for Medical Education and Research.

While cromakalim has established activity as a potassium channel opener and vasodilator, it is substantially insoluble in water. The lipophilicity of cromakalim has limited its usefulness for certain in vivo applications. Cromakalim is often solubilized with DMSO or cremophor, which is also used for the non-water-soluble drug taxol. Cremophor in particular has toxic side effects.

Cromakalim has been tested in human clinical trials which were terminated due to lack of activity, most likely due to poor bioavailability. See for example Donnelly, et. al., “Clinical studies with the potassium channel activator cromakalim in normotensive and hypertensive subjects”, Journal of Cardiovascular Pharmacology, November 1990; 16(5) 790-795.

Given the potential yet unrealized therapeutic benefits of cromakalim, it would be beneficial to have additional methods, compositions and formulations to increase the bioavailability and activity of cromakalim, especially levcromakalim, in vivo which enables additional medical therapy.

### SUMMARY OF THE INVENTION

The present invention provides new medical uses and extended-release formulations for cromakalim, prodrugs of cromakalim, and pharmaceutically acceptable salts thereof of Formula I, II or III:

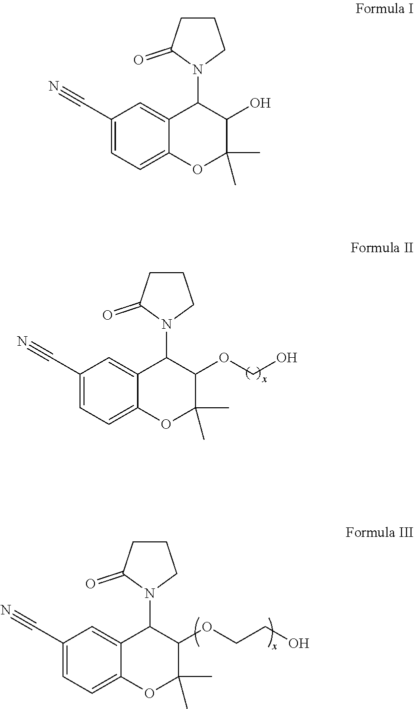

Pharmaceutically acceptable salts of cromakalim (Formula I) include:

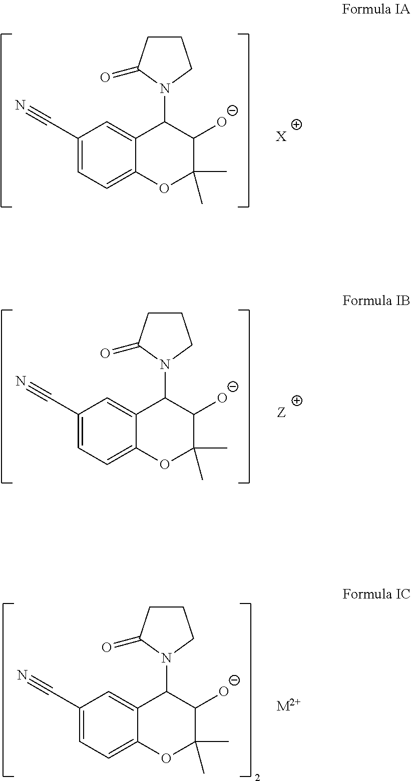

wherein X⁺ and M²⁺ can be any pharmaceutically acceptable cation that achieves the desired results and Z⁺ represents a mixed salt of X⁺.

In certain embodiments, cromakalim can be administered in an extended-release biodegradable polymeric formulation as a mixture of enantiomers and/or a mixture of salts. For example, Adderall is a paradigm example of a mixture of enantiomers and mixed salts of an approved drug. The mixture has equal parts racemic amphetamine and dextroamphetamine salt mixtures (sulfate, aspartate, and saccharate) which results in an approximately 3:1 ratio between the dextroamphetamine and levoamphetamine. The two enantiomers are different enough to give Adderall an effect profile different from the racemate or the d-enantiomer. Likewise, in the present invention cromakalim can be used as any mixture of levcromakalim and dexcromakalim or racemic cromakalim and as a ratio of salts. In Formula IB, Z⁺ is intended to represent a mixed salt cation of X⁺.

While sometimes drawn without stereochemistry at the C—OH position, the cromakalim in any of the presented formulas typically has the levcromakalim stereochemistry. In alternative embodiments, the cromakalim may be used as a mixture of enantiomers, including a racemic form.

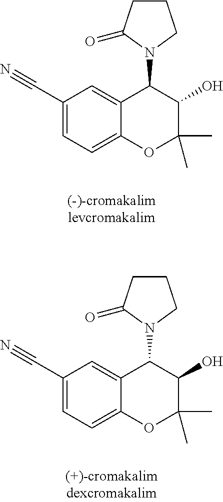 [SMILES: CC1(C)Oc2ccc(C#N)cc2[C@@H](N2CCCC2=O)[C@@H]1O ; CC1(C)Oc2ccc(C#N)cc2[C@H](N2CCCC2=O)[C@H]1O]

In certain embodiments, the cation is selected from sodium, potassium, aluminum, calcium, magnesium, lithium, iron, zinc, arginine, chloroprocaine, choline, diethanolamine, ethanolamine, lysine, histidine, meglumine, procaine, hydroxyethyl pyrrolidine, ammonium, tetrapropylammonium, tetrabutylphosphonium, methyldiethanamine, and triethylamine.

In one embodiment, X⁺ is Na⁺ or K⁺. In one embodiment, X⁺ is Li⁺. In one embodiment, X⁺ is Cs⁺. In one embodiment, X⁺ is an ammonium ion with a net positive charge of one. Non-limiting examples of ammonium ions with a net positive charge of one include:

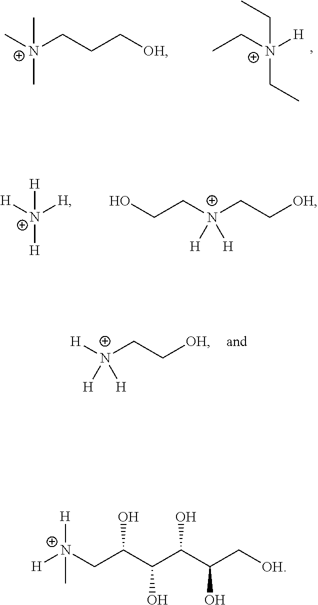

In an alternative embodiment, the ammonium ion with a net positive charge of one has the formula below:

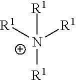

wherein R¹ is C₁-C₆alkyl, for example, but not limited to, methyl, ethyl, propyl, isopropyl, butyl, tbutyl, sec-butyl, isobutyl, —CH₂C(CH₃)₃, —CH(CH₂CH₃)₂, and —CH₂CH(CH₂CH₃)₂, cyclopropyl, CH₂-cyclopropyl, cyclobutyl, and CH₂-cyclobutyl, or aryl, for example, phenyl or napthyl wherein the C₁-C₆alkyl or aryl can be optionally substituted, for example with a hydroxyl group. In one embodiment, the ammonium ion is

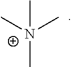

M²⁺, for example, may be, but is not limited to an alkaline earth metal cation (magnesium, calcium, or strontium), a metal cation with an oxidation state of +2 (for example, zinc or iron), or an ammonium ion with a net positive charge of two (for example, benzathine, hexamethyl diammonium, and ethylenediamine). In one embodiment, M²⁺ is Mg²⁺. In one embodiment, M²⁺ is Ca²⁺. In one embodiment, M²⁺ is Sr²⁺. In one embodiment, M²⁺ is Zn²⁺. In one embodiment, M²⁺ is Fe²⁺. In one embodiment, M²⁺ is an ammonium ion with a net positive charge of two. Non-limiting examples of ammonium ions with a net positive charge of two include:

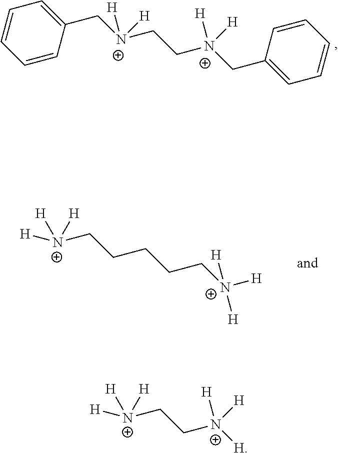

In an alternative embodiment, the ammonium ion with a net positive charge of two has the formula below:

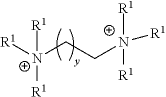

wherein
R¹ is C₁-C₆alkyl, for example, but not limited to, methyl, ethyl, propyl, isopropyl, butyl, tbutyl, sec-butyl, isobutyl, —CH₂C(CH₃)₃, —CH(CH₂CH₃)₂, and —CH₂CH(CH₂CH₃)₂, cyclopropyl, CH₂-cyclopropyl, cyclobutyl, and CH₂-cyclobutyl, or aryl, for example, phenyl or napthyl wherein the C₁-C₆alkyl or aryl can be optionally substituted, for example with a hydroxyl group; and,
y is an integer selected from 1, 2, 3, 4, 5, 6, 7, and 8.

The present invention includes solving the long felt need to enable cromakalim, for example, levcromakalim, therapy by administering a selected compound as described herein in an extended-release formulation, that includes but is not limited to polymeric controlled delivery generally, for example, a microparticle, or nanoparticle, or alternatively a hydrogel or liposome, as further described herein. The fact that cromakalim is lipophilic, which is highly disadvantageous for direct administration and has prevented positive clinical trial results, is converted into an advantage in the present invention when formulated in a lipophilic extended-release formulation such as a microparticle, nanoparticle or liposome. This can enable cromakalim to be used as a therapy for serious diseases as further described.

In certain embodiments, the extended release formulation comprises a biodegradable polymer, including but not limited to poly(lactide-co-glyolide) (PLGA), polylactic acid (PLA), polyglycolic acid (PGA), other aliphatic polyester, poly(caprolactone), polyanhydride, polyamide, polyaminoacid, poly(ester amide), poly(phosphoester), poly(orthoester), hyaluronic acid or polydioxanone. In some embodiments, the biodegradable polymer is end capped with polyethylene glycol (PEG).

The term “microparticle” as used herein means a particle whose size is measured in micrometers (μm). Typically, the microparticle has an average diameter of from about 1 μm to about 100 μm. In some embodiments, the microparticle has an average diameter of from about 0.5 μm to 80 μm, for instance from about 1 μm to 75 μm; from about 10 μm to 75 μm; from about 20 μm to 60 μm; from about 25 μm to 50 μm, and more generally, at least about 0.5, 1, 10, 20, 30, 40, 50, 60, 70, 80, 90 or 100 μm. As used herein, the term “microsphere” means a substantially spherical microparticle.

The term nanoparticle refers to a particle whose size is measured in nanometers, including but not limited to 0.5 nanometers to 100 nanometers, and including at least about 0.5, 1, 10, 20, 30, 40, 50, 60, 70, 80, 90 or 100 nm.

In some embodiment, the extended-release cromakalim formulations are delivered for storage in tissues, including ocular tissues, and are slowly released over time. This slow release from tissues leads to long-term, continuous, and controlled dosing of active cromakalim, and in one embodiment, levcromakalim, following administration of the extended-release formulation.

Therefore, in one embodiment, the present invention provides the controlled delivery of levcromakalim via the administration of an extended-release formulation of a compound Formula I, Formula II, or Formula III or a pharmaceutically acceptable salt thereof to a host, including a human, in need thereof. In one embodiment, the controlled delivery of levcromakalim to the eye is achieved by the topical administration of an extended-release formulation of a compound of the present invention. In select embodiments of the present invention, an extended-release formulation of a compound of Formula I, Formula II, or Formula III or a pharmaceutically acceptable salt thereof is administered to the eye, for example, as a topical drop, and delivers levcromakalim in the eye, for example in the sclera, optic nerve, cornea, iris, ciliary body, trabecular meshwork, and/or the retina.

Extended-release delivery that leads to long-term delivery of the active compound requires less frequent dosing, which is important for patient compliance, adherence, and better outcomes.

Furthermore, the effect of levcromakalim on selected biomarkers for hyperemia and perturbations to vessel integrity has been established. Levcromakalim has no significant impact on the expression of the measured proteins that are indicative of tissue and vessel integrity. The effect of levcromakalim was compared to Y-27632, a Rho kinase inhibitor, which is a class of drugs (exemplified by Rhopressa) that have been shown to have significant side effects caused by perturbations in vessel integrity (e.g., leakiness and vasodilation causing hyperemia, as well as vessel rupture leading to petechia and subconjunctival hemorrhages). Unlike Y-27632, levcromakalim did not significantly alter the protein expression or distribution of these proteins. Therefore, in one embodiment, the use of an extended-release formulation of a compound of Formula I, II or III or a pharmaceutically acceptable salt thereof does not cause significant hyperemia in a patient in need thereof when used during therapy as described further herein, and in some embodiments, over long-term therapy, for example at least one, two, three, four, five, six, or more months. Alternatively, the administration of an extended-release formulation of a compound of Formula I, Formula IL, or Formula III does not significantly induce the expression of at least one protein independently selected from CD31 and VE-Cadherin.

The extended-release cromakalim formulations or pharmaceutically acceptable salt of Formula I, Formula IL, or Formula III can include a cromakalim moiety that is either the (−) (3S,4R)-enantiomer (levcromakalim) or the (+) (3R,4S)-enantiomer (dexcromakalim) or any mixture thereof. The extended-release cromakalim formulations can be formulated with cromakalim as the free acid or a fully or partially neutralized acid. In one embodiment, the pH of the pharmaceutical formulation that includes the extended-release cromakalim formulations or pharmaceutically acceptable salt of Formula I, Formula II, or Formula III is adjusted using a pharmaceutically acceptable base to the desired pH level for pharmaceutical administration, often between about 5.5 or 6.5 and 8.5, and more typically between 6.5 and 8.

The present invention also provides new medical uses for extended-release cromakalim formulations, including blood vessel disorders, cardiovascular disorders, lymphatic diseases, and erectile dysfunction. Extended-release cromakalim formulations when administered systemically can induce peripheral vasodilation. This is a beneficial side effect that can treat blood vessel disorders, such as Raynaud's disease, ischemic limb syndrome, pulmonary arterial hypertension, or sexual disorders, such as erectile dysfunction. Therefore, in one embodiment, the extended-release cromakalim formulation is administered to a host in need thereof, for example a human, for the treatment of Raynaud's disease. In another embodiment, the extended-release cromakalim formulation is administered to a host in need thereof, for example a human, for the treatment of erectile dysfunction.

The invention includes at least the following aspects:
(i) New extended-release compositions of a compound of Formula I, II or III or a pharmaceutically acceptable salt thereof, wherein the extended-release formulation, in certain embodiments, is a microparticle, or nanoparticle, or alternatively a hydrogel or liposome;
(ii) New medical uses that administer an effective amount of an extended-release formulation of a compound of Formula I, II or III or a pharmaceutically acceptable salt thereof to treat a disorder in a host in need thereof,
(iii) Long term medical therapy, including but not limited to ocular therapy (i.e., for at least 6 weeks, 7 weeks, or at least 2, 3, 4, 5, or 6 months or indefinitely for the duration of the therapy) to a host in need thereof, for example, normal tension glaucoma, that includes the administration of an effective amount of an extended-release formulation of a compound of Formula I, II or III as described herein or a pharmaceutically acceptable salt thereof in a manner that does not create significant tachyphylaxis (i.e., loss of activity over time), or alternatively, which does not induce tolerance;
(iv) Extended release human dosing using an effective amount of an extended-release formulation of a compound of Formula I, II or III as described herein or a pharmaceutically acceptable salt thereof to treat glaucoma associated with elevated intraocular pressure, including but not limited to primary open angle glaucoma (POAG), primary angle closure glaucoma (also known as chronic open angle glaucoma, chronic simple glaucoma and glaucoma simplex), pediatric glaucoma, pseudo-exfoliative glaucoma, pigmentary glaucoma, traumatic glaucoma, neovascular glaucoma, irido corneal endothelial glaucoma (ICE), and in an alternative embodiment, uveitic glaucoma, steroid induced glaucoma, and acute glaucoma resulting from advanced cataracts and/or from intravitreal injections;
(v) Ocular therapy using an effective amount of an extended-release formulation of a compound of Formula I, II or III as described herein or a pharmaceutically acceptable salt thereof that does not result in significant hyperemia (which can result in “red eye”, vascular congestion, small bleeds, small punctate bleeds or microhemorrhages) to a host in need thereof,
(vi) A method of treatment that includes an effective amount of an extended-release formulation of a compound of Formula I, II or III as described herein or a pharmaceutically acceptable salt thereof either as primary or secondary or adjunctive treatment as part of the protocol for MIGS (Microinvasive Glaucoma Surgery), including but not limited to miniature versions of trabeculectomy (microtrabeculectomies), trabecular bypass surgeries, totally internal or suprachoroidal shunts, milder/gentler versions of laser cyclo photocoagulation, and in an alternative embodiment, Schlemm's canal stents that dilate Schlemm's canal, goniotomies, canaloplasties, and laser trabeculoplasties;
(vii) Formulations for systemic delivery that include an effective amount of an extended-release formulation of a compound of Formula I, II or III as described herein or a pharmaceutically acceptable salt thereof for ocular therapy to a host in need thereof,
(viii) Formulations for topical delivery that include an effective amount of an extended-release formulation of a compound of Formula I, II or III as described herein or a pharmaceutically acceptable salt thereof for ocular therapy to a host in need thereof,
(ix) Formulations for parenteral delivery that include an effective amount of an extended-release formulation of a compound of Formula I, II or III as described herein or a pharmaceutically acceptable salt thereof for ocular therapy to a host in need thereof,
(x) Formulations for localized delivery that include an effective amount of an extended-release formulation of a compound of Formula I, II or III as described herein or a pharmaceutically acceptable salt thereof for ocular therapy to a host in need thereof,
(xi) Formulations that include an effective amount of an extended-release formulation of a compound of Formula I, II or III as described herein or a pharmaceutically acceptable salt thereof for dermatological or transdermal applications for a host in need thereof;
(xii) The administration of an effective amount of an extended-release formulation of a compound of Formula I, II or III as described herein or a pharmaceutically acceptable salt thereof to treat a cardiovascular disorder in a host such as high blood pressure, congestive heart failure, transient ischemic attack, heart attack, acute myocardial infarction, acute and chronic myocardial ischemia, unstable angina or associated chest pain, arrhythmias, or pulmonary arterial hypertension (PAH), a cardioprotective agent in a host experiencing a heart attack or undergoing heart surgery, a cardioprotective agent for the preservation of heart prior to organ donation, microvascular dysfunction, or endothelial dysfunction;
(xiii) The administration of an effective amount of an extended-release formulation of a compound of Formula I, II or III as described herein or a pharmaceutically acceptable salt thereof to treat a blood vessel disorder, such as Raynaud's disease, peripheral artery disease, including chronic and acute limb ischemia as well as chronic cold hands and/or feet, in a host in need thereof,
(xiv) The administration of an effective amount of an extended-release formulation of a compound of Formula I, II or III as described herein or a pharmaceutically acceptable salt thereof to treat an endocrine disorder such as hypoglycemia, hyperinsulinism or diabetes in a host;
(xv) The administration of an effective amount of an extended-release formulation of a compound of Formula I, II or III as described herein or a pharmaceutically acceptable salt thereof to treat a skeletal muscle disorder such as skeletal muscle myopathy in a host;
(xvi) The administration of an effective amount of an extended-release formulation of a compound of Formula I, II or III as described herein or a pharmaceutically acceptable salt thereof to treat a urology disorder such as erectile dysfunction or female sexual arousal disorder;
(xvii) The administration of an effective amount of an extended-release formulation of a compound of Formula I, II or III as described herein or a pharmaceutically acceptable salt thereof to treat a dermatology disorder such as hypotrichosis (failure to have normal eyelash growth) or baldness in a host in need thereof,
(xviii) The administration of an effective amount of an extended-release formulation of a compound of Formula I, II or III as described herein or a pharmaceutically acceptable salt thereof to treat a neurological disorder such as neuropathic pain or neurodegenerative disease (for example Parkinson's disease and Huntington's disease) in a host in need thereof;
(xix) The administration of an effective amount of an extended-release formulation of a compound of Formula I, II or III as described herein or a pharmaceutically acceptable salt thereof to treat a lymphatic disease such as lymphedema, lymphangitis, lymphadenitis, lymphangiomatosis, Castleman's disease, or a cancer of the lymph system, including Hodgkin's lymphoma, non-Hodgkin's lymphoma, or lymphangiomatosis, in a host in need thereof,
(xx) The administration of an effective amount of an extended-release formulation of a compound of Formula I, II or III as described herein or a pharmaceutically acceptable salt thereof to treat an ocular lymphatic disease selected from conjunctival myxoma, dry eye, conjunctival lymphangiectasia, chemosis, mustard gas keratitis, corneal inflammation, orbital cellulitis, chalazion, dermatochalasis, and blepharochalasis;
(xxi) The administration of an effective amount of an extended-release formulation of a compound Formula I, II or III as described herein or a pharmaceutically acceptable salt thereof to treat tumor hypoperfusion or hypoxia in a host in need thereof,
(xxii) The administration of an effective amount of an extended-release formulation of a compound of Formula I, II or III as described herein or a pharmaceutically acceptable salt thereof to treat a mitochondrial disorder;
(xxiii) The administration of an effective amount of an extended-release formulation of a compound of Formula I, II or III as described herein or a pharmaceutically acceptable salt thereof to treat an ocular disorder in a host such as Graves' ophthalmopathy, thyroid-associated orbitopathy (TAO), Graves' orbitopathy (GO), retrobulbar tumors, cavernous sinus thrombosis, orbital vein thrombosis, episcleral/orbital vein vasculitis, superior vena cava obstruction, superior vena cava thrombosis, carotid cavernous sinus fistula, dural cavernous sinus shunts, orbital varices, central retinal vein occlusion (CRVO), branch retinal vein occlusion (BRVO), artery occlusive/embolic and or hypoperfusion diseases, optic nerve damage due to ischemia (posterior and anterior ischemic optic neuropathy (NAION);
(xxiv) A method of providing cellular protection and/or neuroprotection comprising administering an effective amount of an extended-release formulation of a compound of Formula I, II or III as described herein or a pharmaceutically acceptable salt thereof to a host in need thereof,
(xxv) The administration of an effective amount of an extended-release formulation of a compound of Formula I, II or III as described herein or a pharmaceutically acceptable salt thereof to treat Sturge-Weber Syndrome, including but not limited to Sturge-Weber Syndrome-induced glaucoma in a host in need thereof, and
(xxvi) A pharmaceutical composition comprising an effective amount of an extended-release formulation of a compound of Formula I, II or III as described herein or a pharmaceutically acceptable salt thereof to treat any one of the disorders or diseases described in embodiments (i)-(xxii).

### DETAILED DESCRIPTION OF THE INVENTION

### I. Extended-Release Cromakalim Formulations and their Pharmaceutically Acceptable Salts for Medical Uses as Described Herein

In one aspect, the invention is new medical uses for extended-release cromakalim formulations of a compound and pharmaceutically acceptable salts thereof of Formula I, II or III:

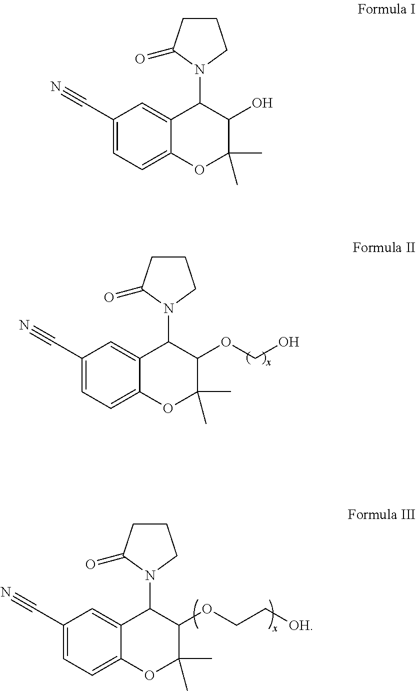

The present invention includes advantageous extended-release formulations that exhibit improved pharmacokinetic properties that lead to long-term, controlled delivery of cromakalim, and in one embodiment, levcromakalim.

Pharmaceutically acceptable salts of cromakalim (Formula I) include:

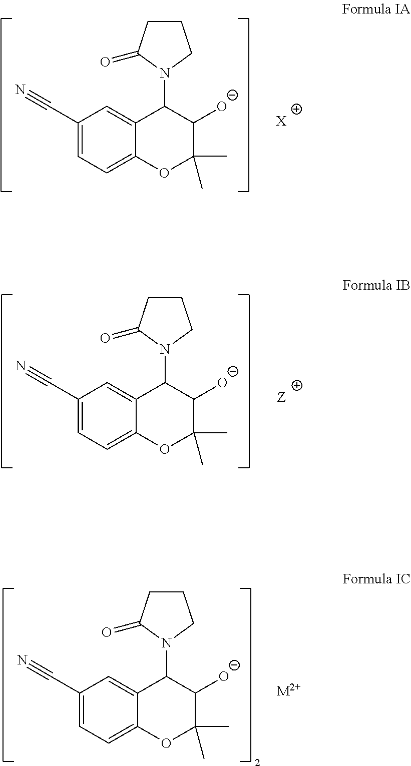

wherein X⁺ and M²⁺ can be any pharmaceutically acceptable cation that achieves the desired results.

In certain embodiments, the cation is selected from sodium, potassium, aluminum, calcium, magnesium, lithium, iron, zinc, arginine, chloroprocaine, choline, diethanolamine, ethanolamine, lysine, histidine, meglumine, procaine, hydroxyethyl pyrrolidine, ammonium, tetrapropylammonium, tetrabutylphosphonium, methyldiethanamine, and triethylamine.

In one embodiment, X⁺ is Na⁺ or K⁺. In one embodiment, X⁺ is Li⁺. In one embodiment, X⁺ is Cs⁺. In one embodiment, X⁺ is an ammonium ion with a net positive charge of one. Non-limiting examples of ammonium ions with a net positive charge of one include:

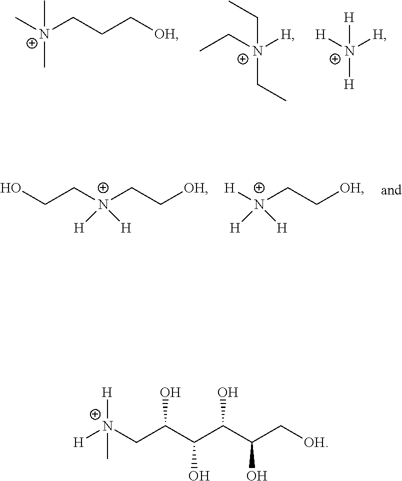

In an alternative embodiment, the ammonium ion with a net positive charge of one has the formula below:

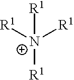

wherein R¹ is C₁-C₆alkyl, for example, but not limited to, methyl, ethyl, propyl, isopropyl, butyl, tbutyl, sec-butyl, isobutyl, —CH₂C(CH₃)₃, —CH(CH₂CH₃)₂, and —CH₂CH(CH₂CH₃)₂, cyclopropyl, CH₂-cyclopropyl, cyclobutyl, and CH₂-cyclobutyl, or aryl, for example, phenyl or napthyl wherein the C₁-C₆alkyl or aryl can be optionally substituted, for example with a hydroxyl group. In one embodiment, the ammonium ion is

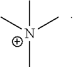

M²⁺, for example, may be, but is not limited to an alkaline earth metal cation (magnesium, calcium, or strontium), a metal cation with an oxidation state of +2 (for example, zinc or iron), or an ammonium ion with a net positive charge of two (for example, benzathine, hexamethyl diammonium, and ethylenediamine). In one embodiment, M²⁺ is Mg²⁺. In one embodiment, M²⁺ is Ca²⁺. In one embodiment, M²⁺ is Sr²⁺. In one embodiment, M²⁺ is Zn²⁺. In one embodiment, M²⁺ is Fe²⁺. In one embodiment, M²⁺ is an ammonium ion with a net positive charge of two. Non-limiting examples of ammonium ions with a net positive charge of two include:

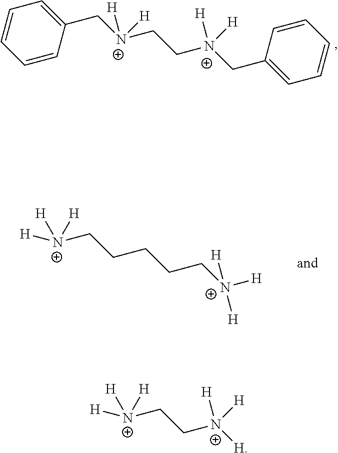

In an alternative embodiment, the ammonium ion with a net positive charge of two has the formula below:

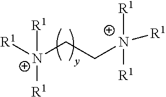

wherein
R¹ is C₁-C₆alkyl, for example, but not limited to, methyl, ethyl, propyl, isopropyl, butyl, tbutyl, sec-butyl, isobutyl, —CH₂C(CH₃)₃, —CH(CH₂CH₃)₂, and —CH₂CH(CH₂CH₃)₂, cyclopropyl, CH₂-cyclopropyl, cyclobutyl, and CH₂-cyclobutyl, or aryl, for example, phenyl or napthyl wherein the C₁-C₆alkyl or aryl can be optionally substituted, for example with a hydroxyl group; and,
y is an integer selected from 1, 2, 3, 4, 5, 6, 7, and 8.

In certain embodiments, the cation is a lipophilic cation that is more compatible with a lipophilic extended-release formulation than a metal cation. Examples are quaternary amines, including choline.

Non-limiting examples of a compound of Formula IA include:

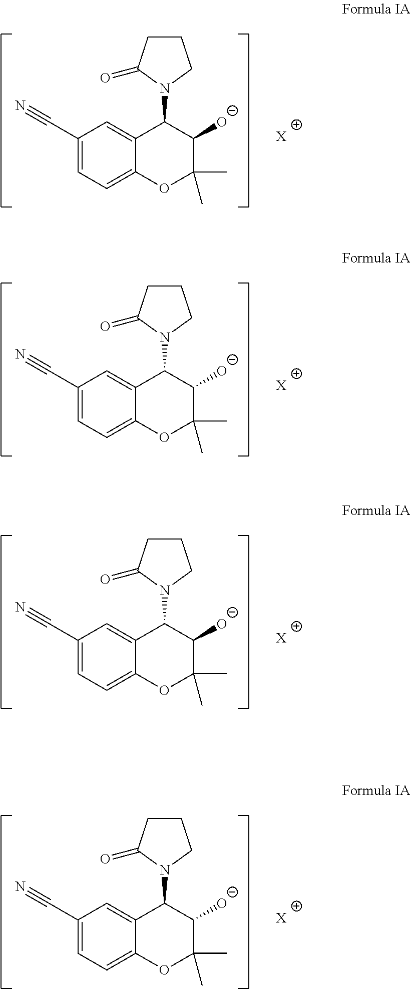

Non-limiting examples of a compound of Formula IB include:

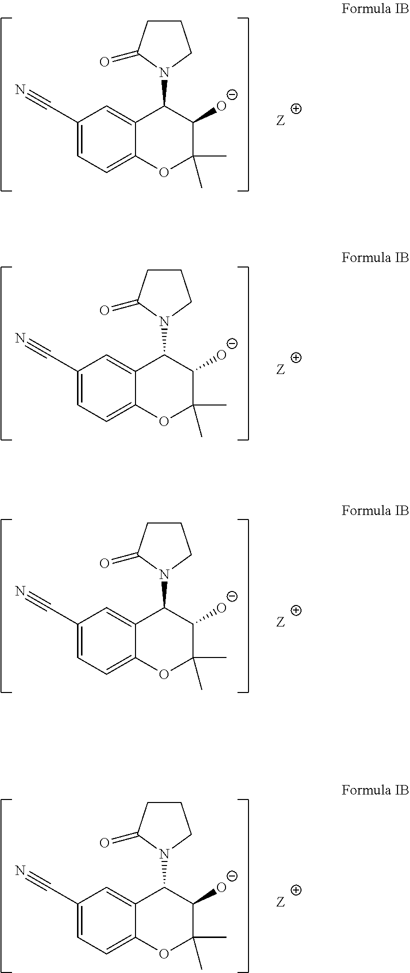

Non-limiting examples of a compound of Formula IC include:

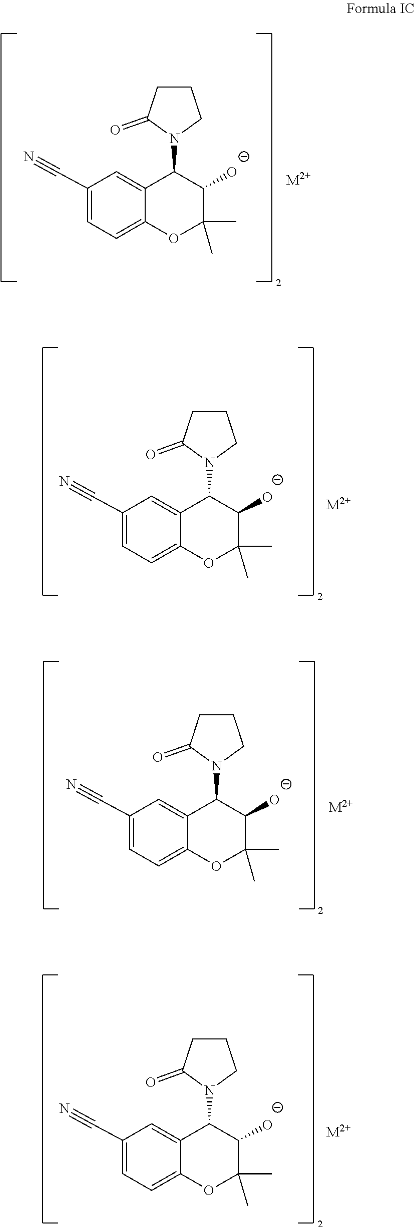

Pharmaceutically acceptable salt of Formula II include:

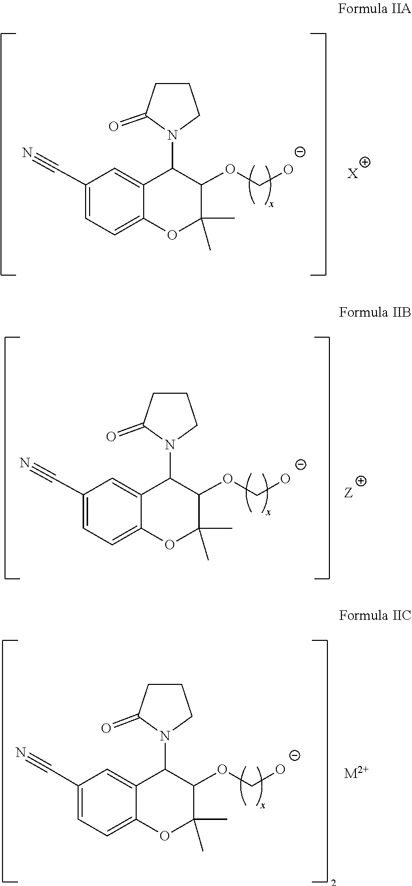

wherein X⁺ and M²⁺ are as defined above; and
x is an integer selected from 1, 2, 3, 4, or 5.

Non-limiting examples of a compound of Formula IIA include:

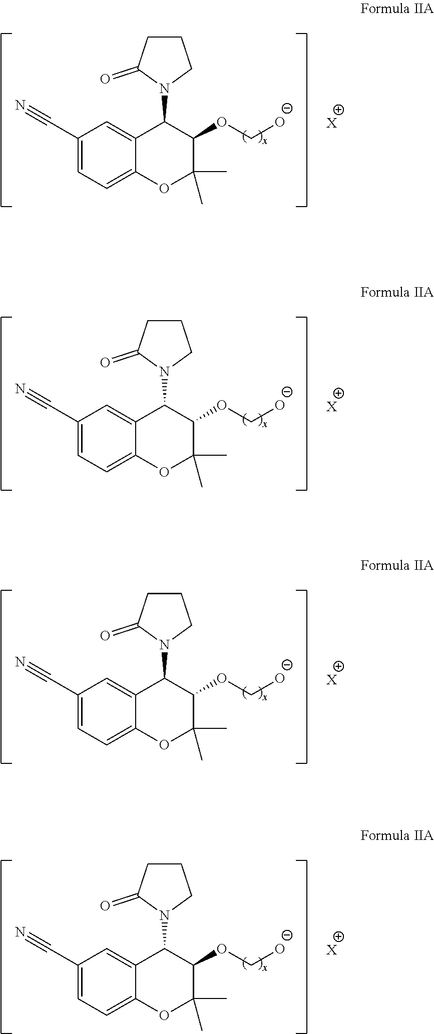

In one embodiment of Formula IIA, x is 1.

In one embodiment of Formula IIA, x is 2.

In one embodiment of Formula IIA, x is 3.

In one embodiment of Formula IIA, x is 4.

In one embodiment of Formula IIA, x is 5.

Non-limiting examples of a compound of Formula IIB include:

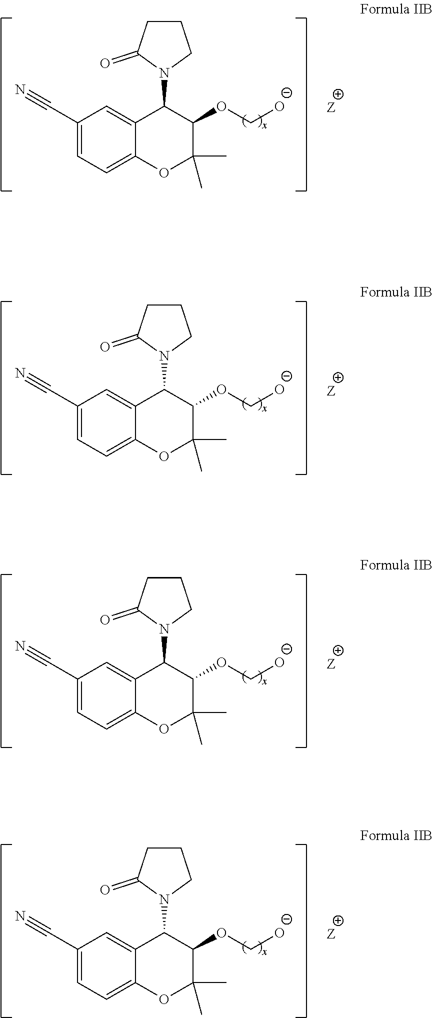

In one embodiment of Formula IIB, x is 1.

In one embodiment of Formula IIB, x is 2.

In one embodiment of Formula IIB, x is 3.

In one embodiment of Formula IIB, x is 4.

In one embodiment of Formula IIB, x is 5.

Non-limiting examples of a compound of Formula IIC include:

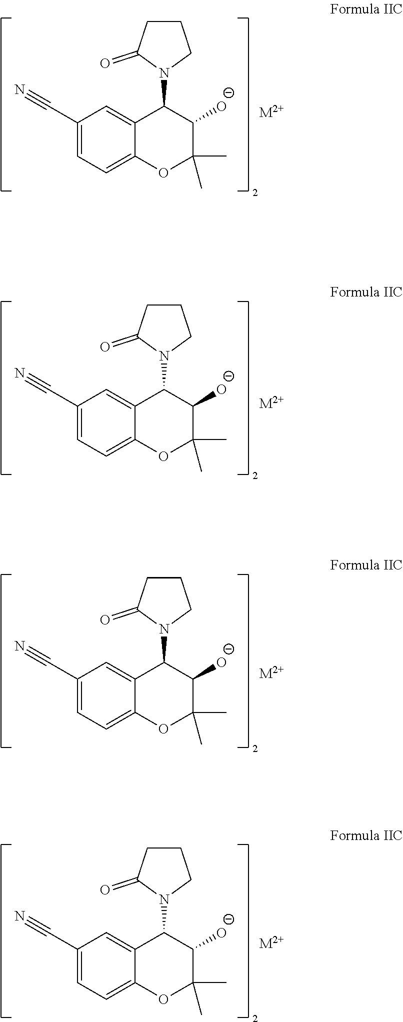

In certain embodiments x is 1.

In certain embodiments x is 2.

In certain embodiments x is 3.

In certain embodiments x is 4.

In certain embodiments x is 5.

In one embodiment of Formula IIC, x is 1.

In one embodiment of Formula IIC, x is 2.

In one embodiment of Formula IIC, x is 3.

In one embodiment of Formula IIC, x is 4.

In one embodiment of Formula IIC, x is 5.

Pharmaceutically acceptable salt of Formula III include:

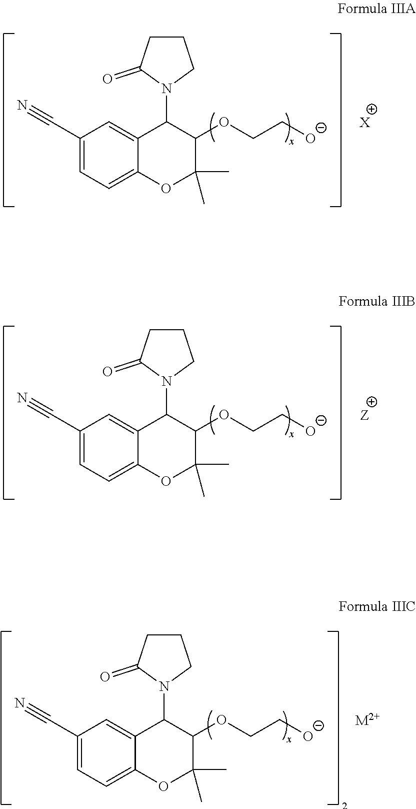

Non-limiting examples of a compound of Formula IIIA include:

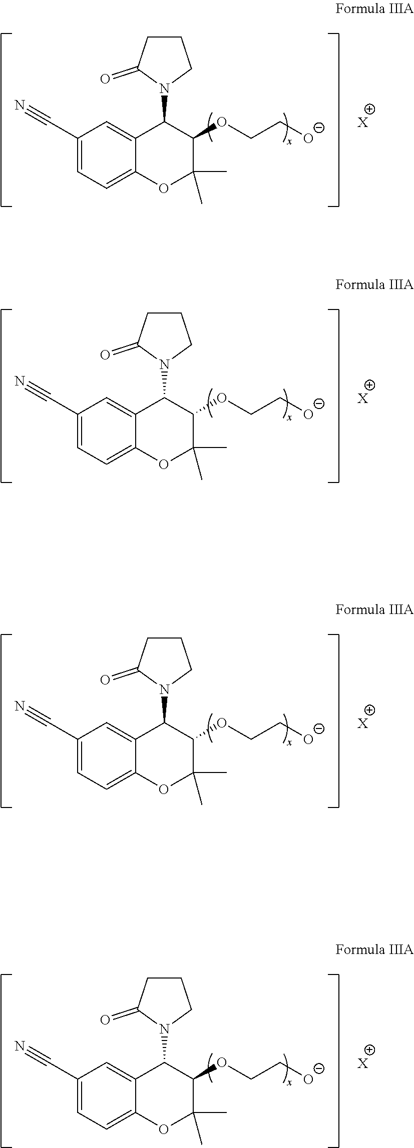

In one embodiment of Formula IIIA, x is 1.

In one embodiment of Formula IIIA, x is 2.

In one embodiment of Formula IIIA, x is 3.

In one embodiment of Formula IIIA, x is 4.

In one embodiment of Formula IIIA, x is 5.

Non-limiting examples of a compound of Formula IIIB include:

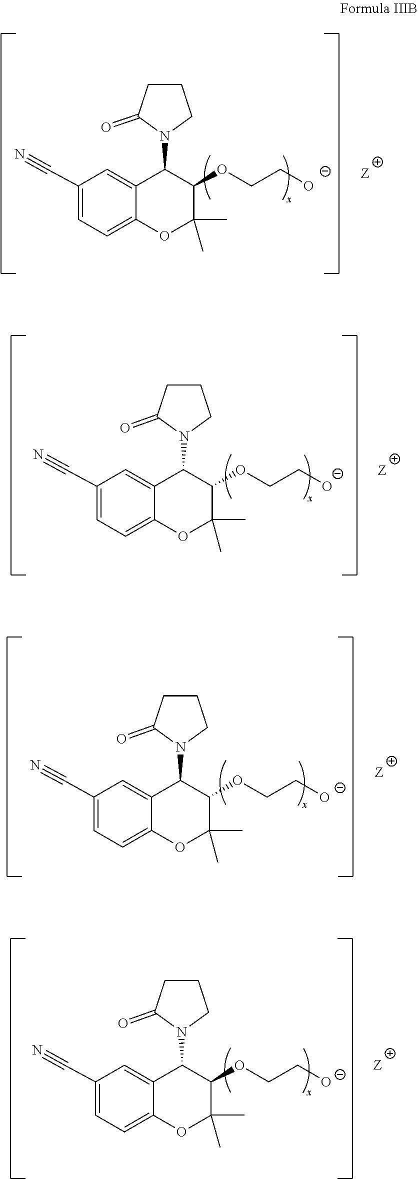

In one embodiment of Formula IIIB, x is 1.

In one embodiment of Formula IIIB, x is 2.

In one embodiment of Formula IIIB, x is 3.

In one embodiment of Formula IIIB, x is 4.

In one embodiment of Formula IIIB, x is 5.

Non-limiting examples of a compound of Formula IIIC include:

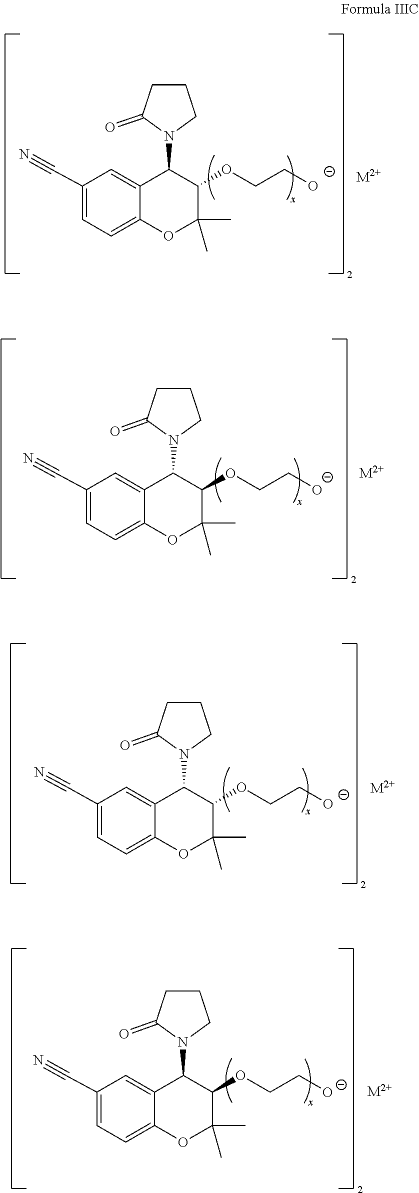

In one embodiment of Formula IIIC, x is 1.

In one embodiment of Formula IIIC, x is 2.

In one embodiment of Formula IIIC, x is 3.

In one embodiment of Formula IIIC, x is 4.

In one embodiment of Formula IIIC, x is 5.

The use of a selected pharmaceutically acceptable salt in the extended release formulation such as described above may be useful in medical treatments. In general, a pharmaceutically acceptable salt can increase or decrease the effectiveness or toxicity of a drug or can change its pharmacokinetics or its distribution in the body through tissues. For example, one pharmaceutically acceptable salt may concentrate in an organ, and another salt may concentrate in a different organ. As another example, increased water solubility alone does not guarantee that a compound will penetrate the eye after release from the controlled release formulation, reach the relevant site of action, achieve sufficient in vivo concentrations, or have a beneficial pharmacologic effect. For ocular topical dosing, after release, drugs have to reside on the surface of the eye long enough to penetrate the eye. This requires either administering the extended release formulation to the desired site or releasing the drug in a manner that allows it to traverse multiple layers of the ocular surface, including the tear film, the cornea, the conjunctiva, and the sclera, which all have varying degrees of hydrophilicity and hydrophobicity due to cell membranes, cell junctions, and the aqueous, lipid, and protein components of the tear film. Topical dosing is made more complicated by the constant renewing and washing of the ocular surface via the tear that in turn drain through the nasolacrimal (tear) ducts. For a compound to enter the eye, it must be able to penetrate before it is washed out.

An aspect of the present invention is that administration of a selected disclosed pharmaceutically acceptable salt in an extended-release formulation may achieve a more useful pharmaceutical effect, and in particular wherein the drug can enter relevant tissues or chambers of the eye in an effective amount to achieve efficacy, for example, by entering into the anterior chamber, reaching the trabecular meshwork, into the vitreous humor, or reaching the retina.

Therefore, another aspect of the present invention is that extended release formulation of the present invention allows the delivery through multiple tissues for topical, parenteral, local or systemic delivery generally, as further disclosed herein, in a therapeutic amount in a manner that is consistent over a sufficient length of time to provide a pharmacologic effect on the target tissue to modify the disorder of interest.

### Additional Embodiments of the Present Invention

1. In certain embodiments, an extended-release pharmaceutical composition comprising a biodegradable microparticle, nanoparticle or other polymeric formulation of cromakalim Formula I, Formula II, or Formula III

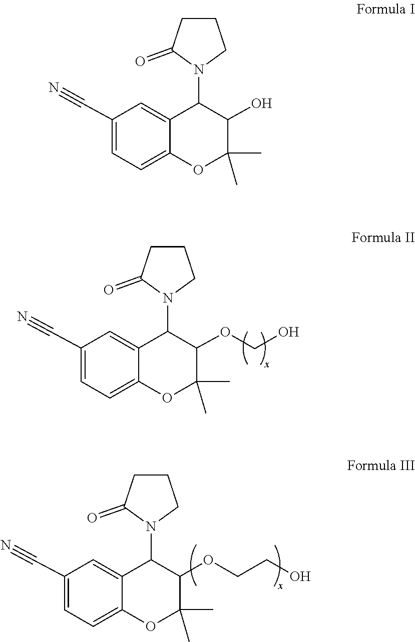

or a pharmaceutically acceptable salt thereof, wherein x is an integer selected from 1, 2, 3, 4, and 5, and wherein cromakalim can be substantially in the levo stereoconfiguration, or a mixture of the levo and dextro configurations, including a racemic mixture is provided.

2. The extended-release pharmaceutical composition of embodiment 1, wherein the compound of Formula I, II or III is formulated in biodegradable microparticles.

3. The extended-release pharmaceutical composition of embodiment 1, wherein the compound of Formula I, II or III is formulated in biodegradable nanoparticles.

4. The extended-release pharmaceutical formulation of embodiment 1, wherein the compound of Formula I, II or III is formulated in a biodegradable polymer.

5. The extended-release pharmaceutical composition of embodiments 1-4, wherein the biodegradable polymer comprises poly(lactide-co-glyolide) (PLGA), polylactic acid (PLA), polyglycolic acid (PGA), other aliphatic polyester, poly(caprolactone), polyanhydride, polyamide, polyaminoacid, poly(ester amide), poly(phosphoester), poly(orthoester), hyaluronic acid, a pluronic polymer, polyvalerolactone, poly(1,3-dioxan-2-one); poly(sebacic anhydride); or polyethylene glycol (PEG).

6. The extended-release pharmaceutical composition of embodiment 5, wherein the biodegradable polymer is end-capped with polyalkylene glycol, a carbohydrate, and or an acyclic polyacetal derived from a polysaccharide.

7. The extended-release pharmaceutical composition of embodiment 5, wherein the biodegradable polymer is not PEG but is end-capped with PEG.

8. The extended-released pharmaceutical composition of embodiments 1-7 wherein Formula I selected from Formula IA, Formula IB, or Formula IC:

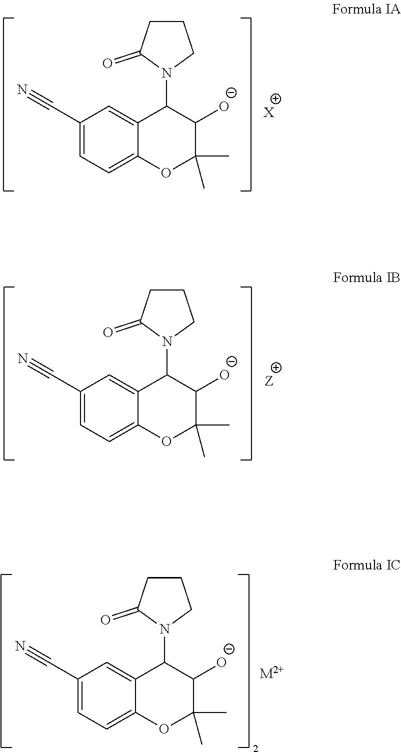

wherein X⁺ and M²⁺ are pharmaceutically acceptable cations and Z⁺ is a mixed salt cation of X⁺.

9. The extended-released pharmaceutical composition of embodiment 8, wherein the X⁺ cation is selected from sodium, potassium, aluminum, calcium, magnesium, lithium, iron, zinc, arginine, chloroprocaine, cesium, choline, diethanolamine, ethanolamine, lysine, histidine, meglumine, procaine, hydroxyethyl pyrrolidine, ammonium, tetrapropylammonium, tetrabutylphosphonium, methyldiethanamine, triethylamine, an ammonium ion of the formula:

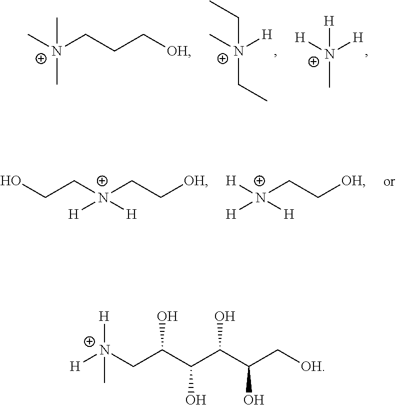

or an ammonium ion of the formula:

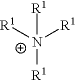

wherein R¹ is C₁-C₆ alkyl, aryl, wherein the C₁-C₆ alkyl or aryl.

10. The extended-released pharmaceutical composition of embodiment 8, wherein M²⁺ is an alkaline earth metal cation, a metal cation or an ammonium ion.

11. In certain embodiments, a method to treat an ocular disorder selected from Graves' ophthalmopathy, cavernous sinus thrombosis, orbital vein vasculitis, carotid-cavernous sinus fistula, orbital varices, central retinal vein occlusion, branch retinal vein occlusion, and non-arteritic anterior ischemic optic neuropathy in a host in need thereof comprising administering an effective amount an extended-release formulation comprising a biodegradable microparticle, nanoparticle or other polymeric formulation of a compound of cromakalim Formula I, Formula IL, or Formula III:

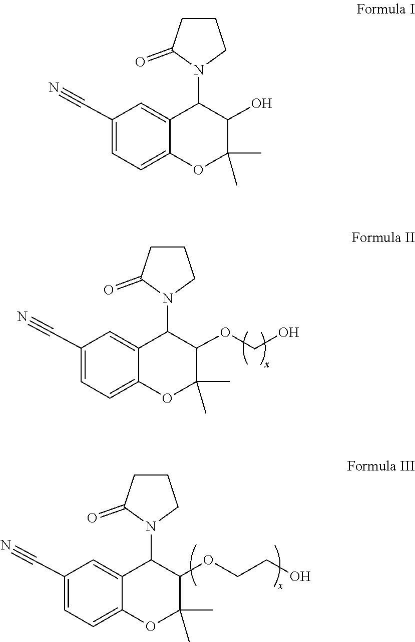

or a pharmaceutically acceptable salt, optionally in a pharmaceutically acceptable carrier, wherein x is an integer selected from 1, 2, 3, 4, and 5, and wherein cromakalim can be substantially in the levo stereoconfiguration, or a mixture of the levo and dextro configurations, including a racemic mixture is provided.

12. The method of embodiment 11, wherein the ocular disorder is non-arteritic anterior ischemic optic neuropathy.

13. In certain embodiments, a method to treat a blood vessel disorder selected from Raynaud's disease, peripheral artery disease, chronic limb ischemia, thrombophlebitis, pulmonary arterial hypertension, and chronic venous insufficiency in a host in need thereof comprising administering an effective amount of an extended-release formulation comprising a biodegradable microparticle, nanoparticle or other polymeric formulation of a compound of cromakalim Formula I, Formula IL, or Formula III:

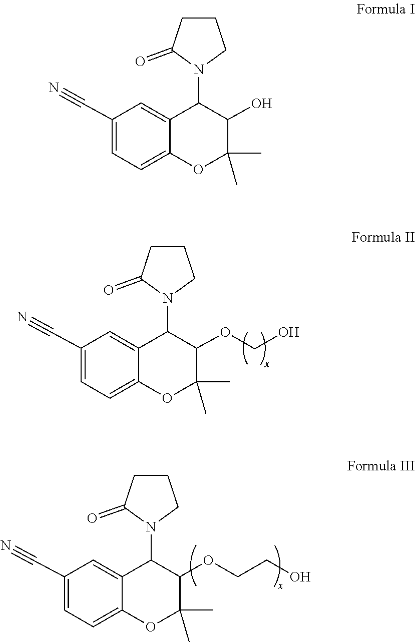

or a pharmaceutically acceptable salt, optionally in a pharmaceutically acceptable carrier, wherein x is an integer selected from 1, 2, 3, 4, and 5, and wherein cromakalim can be substantially in the levo stereoconfiguration, or a mixture of the levo and dextro configurations, including a racemic mixture is provided.

14. The method of embodiment 13, wherein the blood vessel disorder is Raynaud's disease.

15. The method of embodiment 13, wherein the blood vessel disorder is pulmonary arterial hypertension.

16. In certain embodiments, a method to treat a cardiovascular disease selected from chronic or acute myocardial ischemia, microvascular dysfunction, coronary artery disease, arrhythmia, high blood pressure, endothelial dysfunction, and a heart attack in a host in need thereof comprising administering an effective amount of an extended-release formulation comprising a biodegradable microparticle, nanoparticle or other polymeric formulation of a compound of cromakalim Formula I, Formula IL, or Formula III:

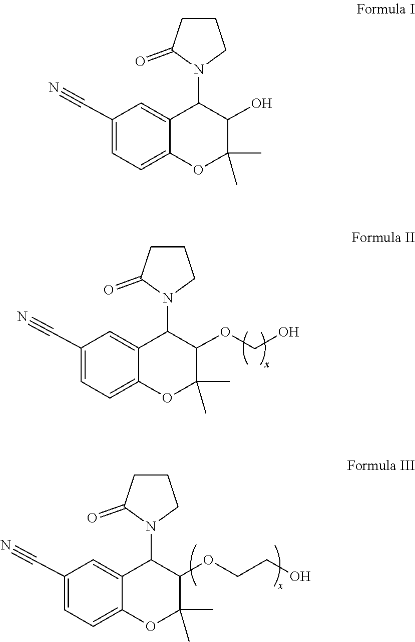

or a pharmaceutically acceptable salt, optionally in a pharmaceutically acceptable carrier, wherein x is an integer selected from 1, 2, 3, 4, and 5, and wherein cromakalim can be substantially in the levo stereoconfiguration, or a mixture of the levo and dextro configurations, including a racemic mixture is provided.

17. In certain embodiments, a method to treat erectile dysfunction or female sexual arousal disorder in a host in need thereof comprising administering an effective amount of an extended-release formulation comprising a biodegradable microparticle, nanoparticle or other polymeric formulation of a compound of cromakalim Formula I, Formula II, or Formula III:

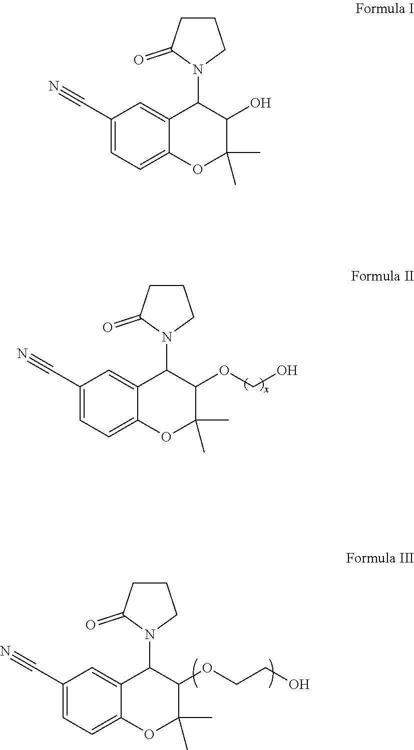

or a pharmaceutically acceptable salt, optionally in a pharmaceutically acceptable carrier, wherein x is an integer selected from 1, 2, 3, 4, and 5, and wherein cromakalim can be substantially in the levo stereoconfiguration, or a mixture of the levo and dextro configurations, including a racemic mixture is provided.

18. The method of embodiment 17 to treat erectile dysfunction.

19. In certain embodiments, a method to treat a lymphatic disease selected from lymphadenopathy, lymphangitis, lymphangiectasia, lymphadenitis, and lymphangiomatosis in a host in need thereof comprising administering an effective amount of an extended-release formulation comprising a biodegradable microparticle, nanoparticle or other polymeric formulation of a compound of cromakalim Formula I, Formula IL, or Formula III:

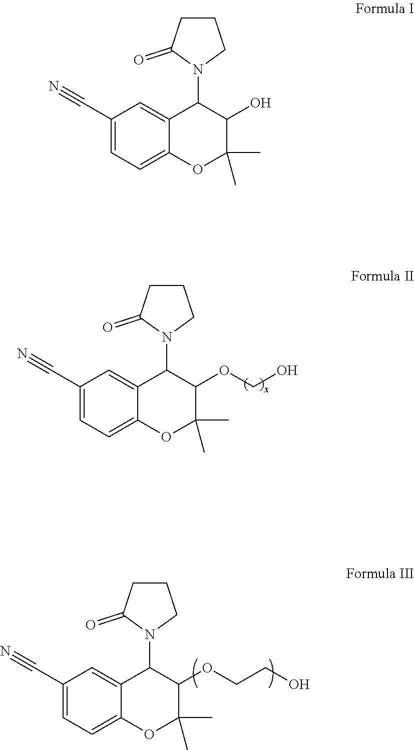

or a pharmaceutically acceptable salt, optionally in a pharmaceutically acceptable carrier, wherein x is an integer selected from 1, 2, 3, 4, and 5, and wherein cromakalim can be substantially in the levo stereoconfiguration, or a mixture of the levo and dextro configurations, including a racemic mixture is provided.

20. In certain embodiments, a method to treat an ocular lymphatic disorder selected from conjunctival myxoma, dry eye, conjunctival lymphangiectasia, chemosis, mustard gas keratitis, corneal inflammation, orbital cellulitis, chalazion, dermatochalasis, and blepharochalasis in a host in need thereof comprising administering an effective amount an extended-release formulation comprising a biodegradable microparticle, nanoparticle or other polymeric formulation of a compound of cromakalim Formula I, Formula II, or Formula III:

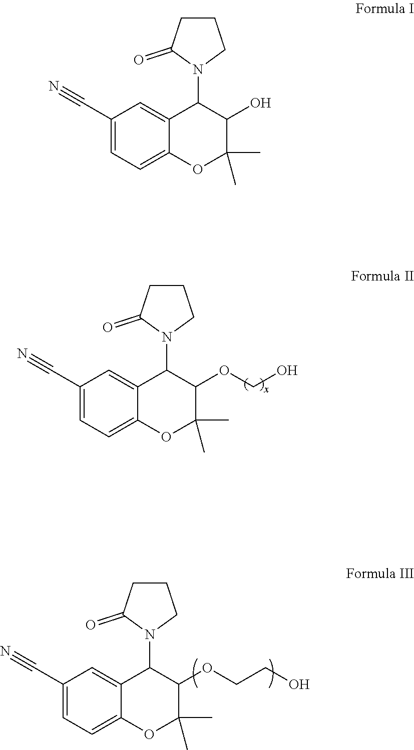

or a pharmaceutically acceptable salt, optionally in a pharmaceutically acceptable carrier, wherein x is an integer selected from 1, 2, 3, 4, and 5, and wherein cromakalim can be substantially in the levo stereoconfiguration, or a mixture of the levo and dextro configurations, including a racemic mixture is provided.

21. The methods of any one of embodiments 10-20, wherein the effective amount of an extended-release formulation of a compound of Formula I, Formula II, or Formula III does not cause significant hyperemia.

22. The methods of any one of embodiment 10-21, wherein the extended-release formulation of a compound is a pharmaceutically acceptable salt of Formula I selected from Formula IA, Formula IB, or Formula IC:

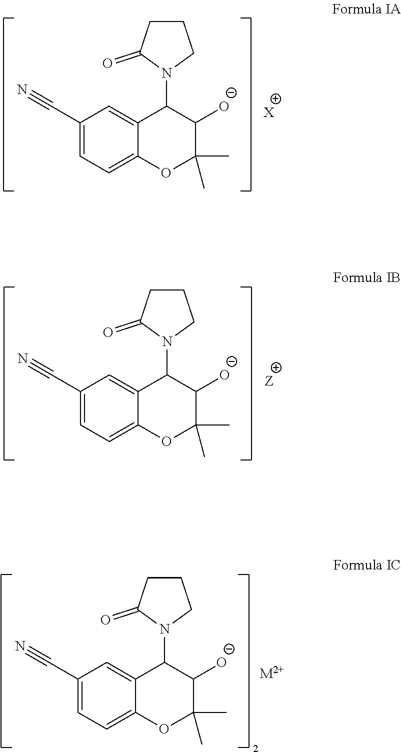

wherein X⁺ and M²⁺ are a pharmaceutically acceptable cation and Z⁺ is a mixed salt cation of X⁺.

23. The method of embodiment 22, wherein the compound of Formula IA is selected from:

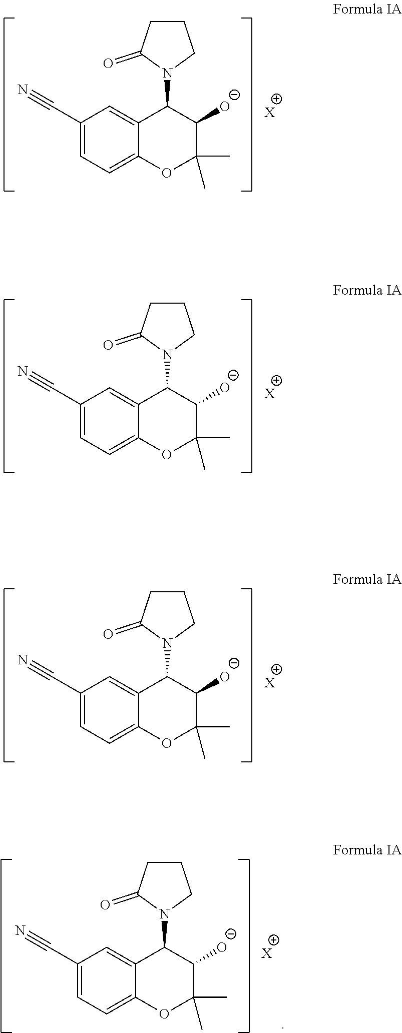

24. The method of embodiment 22, wherein the compound of Formula IB is selected from:

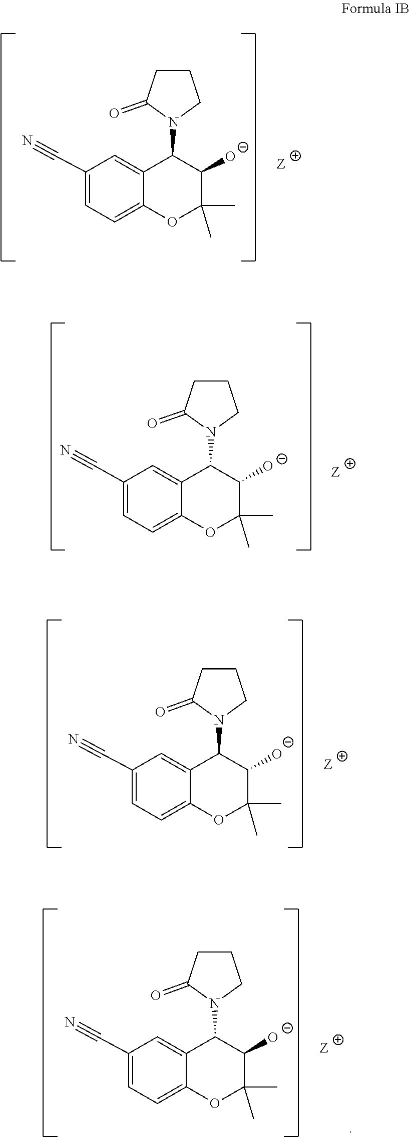

25. The method of embodiment 22, wherein the compound of Formula IC is selected from:

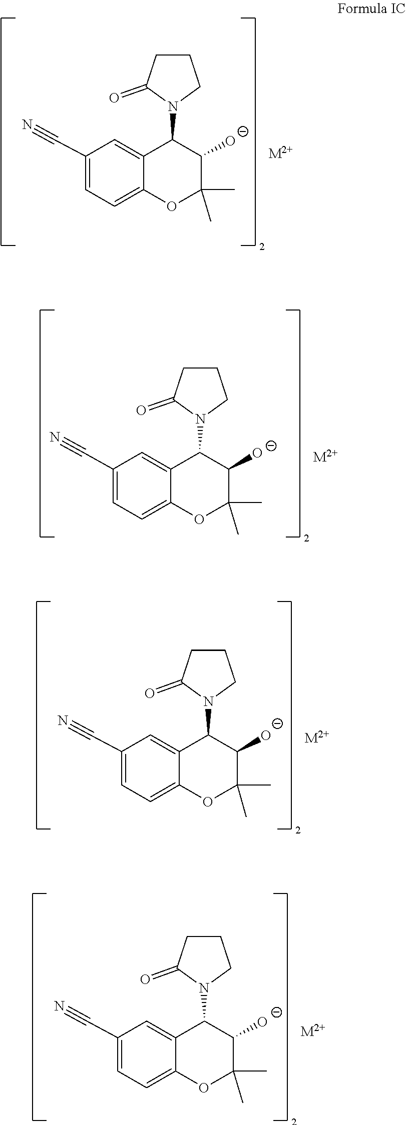

26. The method ofany one of embodiment 10-21, wherein the compound is a pharmaceutically acceptable salt of Formula II selected from Formula IIA, Formula IIB, or Formula IIC:

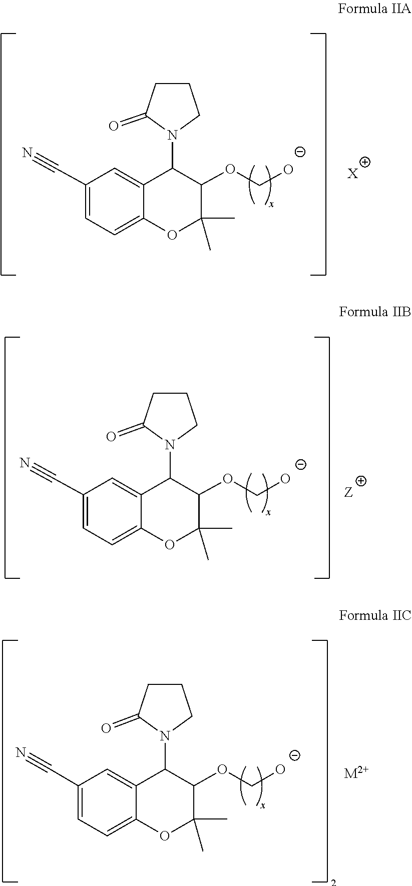

wherein X⁺ and M²⁺ are a pharmaceutically acceptable cation;
Z⁺ is a mixed salt cation of X⁺; and
x is an integer selected from 1, 2, 3, 4, or 5.

27. The method of embodiment 26, wherein the compound is a pharmaceutically acceptable salt of Formula III selected from Formula IIIA, Formula IIIB, or Formula IIIC:

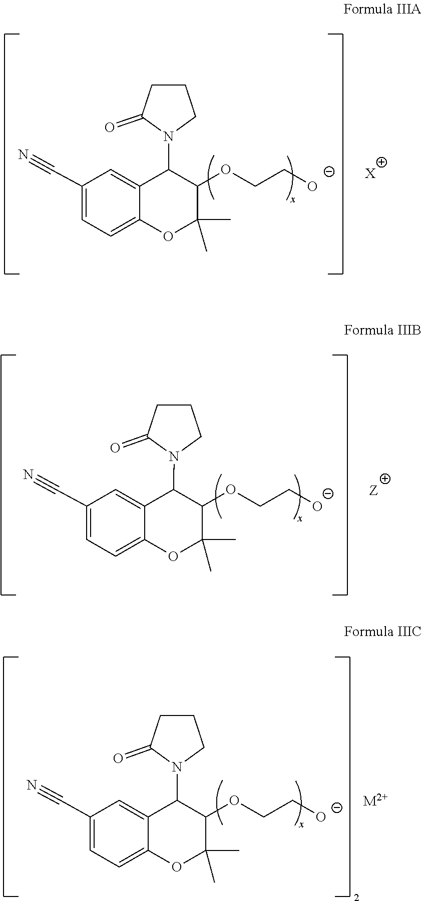

wherein X⁺ and M²⁺ are a pharmaceutically acceptable cation;
Z⁺ is a mixed salt cation of X⁺; and
x is an integer selected from 1, 2, 3, 4, and 5, and wherein the C—OH bond can be substantially in the levo stereoconfiguration, or a mixture of the levo and dextro configurations, including a racemic mixture.

28. The method of any one of embodiments 11-27, wherein the host is a human.

29. In certain embodiments, an extended-release pharmaceutical composition comprising a biodegradable microparticle, nanoparticle or other polymeric formulation of cromakalim Formula I, Formula II, or Formula III

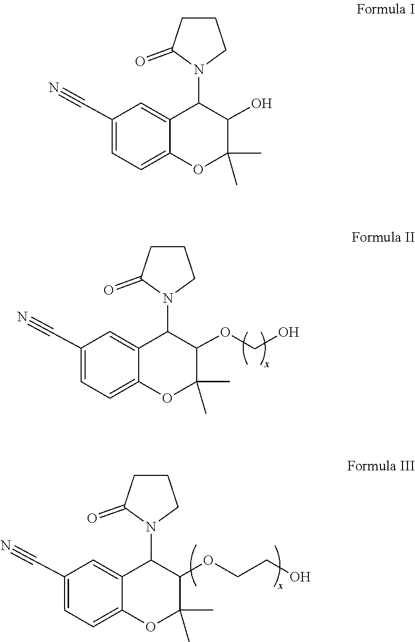

or a pharmaceutically acceptable salt, optionally in a pharmaceutically acceptable carrier, for use to treat an ocular disorder selected from Graves' ophthalmopathy, cavernous sinus thrombosis, orbital vein vasculitis, carotid-cavernous sinus fistula, orbital varices, central retinal vein occlusion, branch retinal vein occlusion, or non-arteritic anterior ischemic optic neuropathy in a host in need thereof is provided.

30. The extended-release pharmaceutical composition of embodiment 29, wherein the ocular disorder is non-arteritic anterior ischemic optic neuropathy.

31. In certain embodiments, an extended-release pharmaceutical composition comprising a biodegradable microparticle, nanoparticle or other polymeric formulation of cromakalim Formula I, Formula II, or Formula III

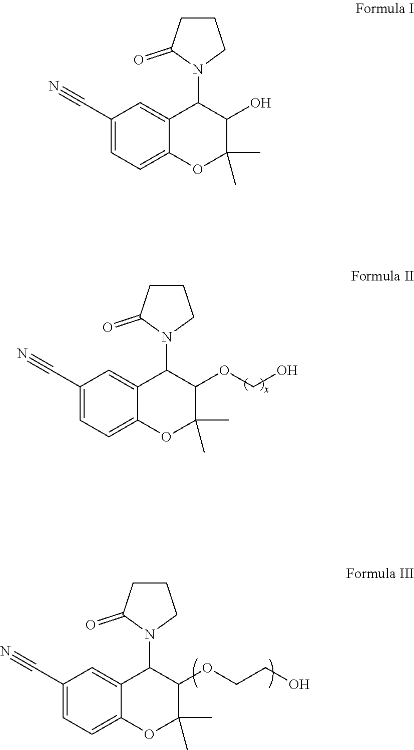

or a pharmaceutically acceptable salt, optionally in a pharmaceutically acceptable carrier, for use to treat a blood vessel disorder selected from Raynaud's disease, peripheral artery disease, chronic limb ischemia, thrombophlebitis, pulmonary arterial hypertension, and chronic venous insufficiency in a host in need thereof wherein x is an integer selected from 1, 2, 3, 4, and 5, and wherein cromakalim can be substantially in the levo stereoconfiguration, or a mixture of the levo and dextro configurations, including a racemic mixture is provided.

32. The extended-release pharmaceutical composition of embodiment 31, wherein the blood vessel disorder is Raynaud's disease.

33. The extended-release pharmaceutical composition of embodiment 31, wherein the blood vessel disorder is pulmonary arterial hypertension.

34. In certain embodiments, an extended-release pharmaceutical composition comprising a biodegradable microparticle, nanoparticle or other polymeric formulation of cromakalim Formula I, Formula II, or Formula III

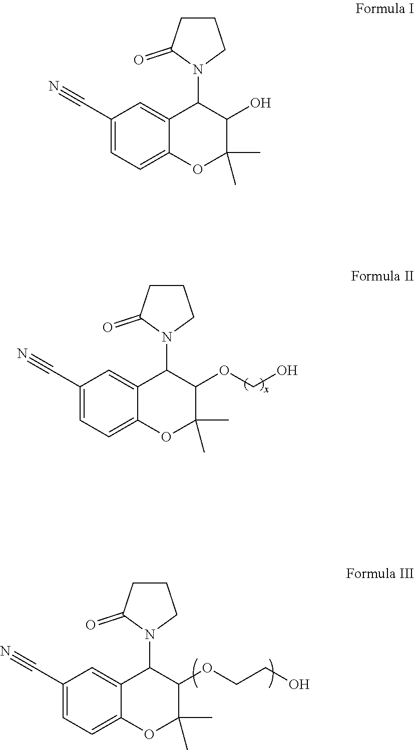

or a pharmaceutically acceptable salt, optionally in a pharmaceutically acceptable carrier, for use to treat a disorder or disease described herein in a host in need thereof wherein x is an integer selected from 1, 2, 3, 4, and 5, and wherein cromakalim can be substantially in the levo stereoconfiguration, or a mixture of the levo and dextro configurations, including a racemic mixture is provided.

35. The use of an effective amount of an extended-release formulation of a compound of cromakalim Formula I, Formula IL, or Formula III,

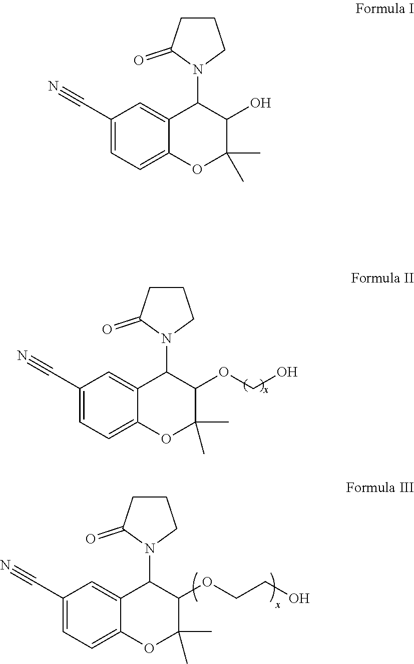

or a pharmaceutically acceptable salt, optionally in a pharmaceutically acceptable carrier, in the manufacture of a medicament for the treatment of an ocular disorder selected from Graves' ophthalmopathy, cavernous sinus thrombosis, orbital vein vasculitis, carotid-cavernous sinus fistula, orbital varices, central retinal vein occlusion, branch retinal vein occlusion, and non-arteritic anterior ischemic optic neuropathy in a host in need thereof wherein x is an integer selected from 1, 2, 3, 4, and 5, and wherein cromakalim can be substantially in the levo stereoconfiguration, or a mixture of the levo and dextro configurations, including a racemic mixture.

36. The use of embodiment 35, wherein the ocular disorder is non-arteritic anterior ischemic optic neuropathy.

37. The use of an effective amount of an extended-release formulation comprising a biodegradable microparticle, nanoparticle or other polymeric formulation of a compound of cromakalim Formula I, Formula IL, or Formula III:

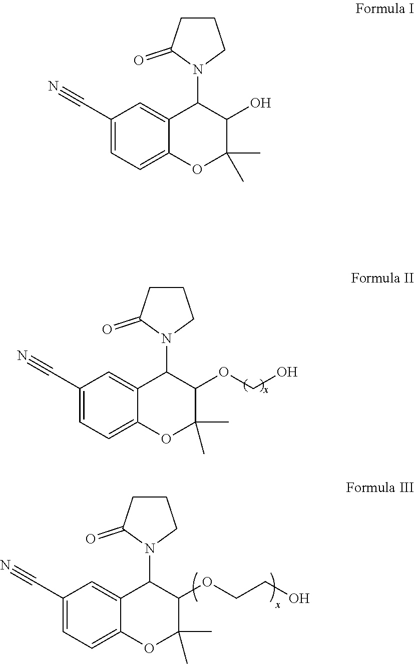

or a pharmaceutically acceptable salt, optionally in a pharmaceutically acceptable carrier, in the manufacture of medicament for the treatment of a blood vessel disorder selected from Raynaud's disease, peripheral artery disease, chronic limb ischemia, thrombophlebitis, pulmonary arterial hypertension, and chronic venous insufficiency in a host in need thereof wherein x is an integer selected from 1, 2, 3, 4, and 5, and wherein the cromakalim can be substantially in the levo stereoconfiguration, or a mixture of the levo and dextro configurations, including a racemic mixture.

38. The use of embodiment 37, wherein the blood vessel disorder is Raynaud's disease.

39. The use of embodiment 37, wherein the blood vessel disorder is pulmonary arterial hypertension.

40. In certain embodiments the use of an effective amount of an extended-release formulation comprising a biodegradable microparticle, nanoparticle or other polymeric formulation of a compound of cromakalim Formula I, Formula II, or Formula III:

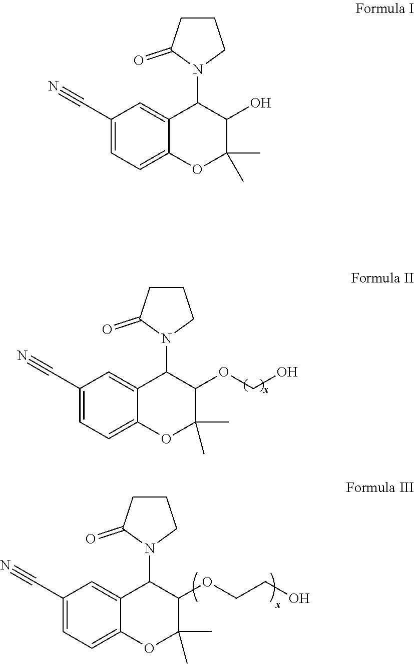

or a pharmaceutically acceptable salt, optionally in a pharmaceutically acceptable carrier, in the manufacture of a medicament for the treatment of a disorder or disease described herein in a host in need thereof wherein x is an integer selected from 1, 2, 3, 4, and 5, and wherein cromakalim can be substantially in the levo stereoconfiguration, or a mixture of the levo and dextro configurations, including a racemic mixture is provided.

41. In certain embodiments, an extended-release pharmaceutical composition comprising a biodegradable microparticle, nanoparticle or other polymeric formulation of cromakalim Formula I, Formula II, or Formula III

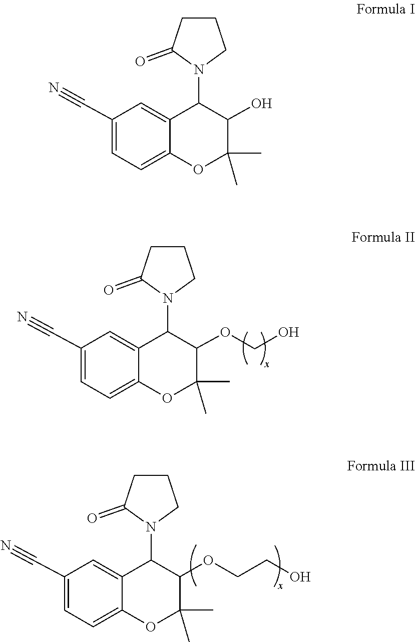

or a pharmaceutically acceptable salt thereof in an effective amount to treat a host using any of the methods described herein is provided.

### II. Medical Uses of Extended-Release Formulations of Compounds of Formulas I, II and III, and in Particular Levcromakalim, or their Pharmaceutically Acceptable Salts

The present invention provides new methods of use and compositions to deliver an effective amount of an extended-release formulation of a compound or a pharmaceutically acceptable salt thereof of Formula I-III, including levcromakalim or its salt. The invention includes at least the following aspects.

A “patient” or “host” or “subject”, as used herein, is typically a human and the method is for human therapy. In appropriate circumstances, the scope may include a non-human animal in need of treatment or prevention of any of the disorders as specifically described herein, for example, a mammal, primate (other than human), cow, sheep, goat, horse, dog, cat, rabbit, rat, mice, bird or the like.

Long Term Therapy without Significant Tachyphylaxis or Tolerance

In one embodiment, the invention includes long term medical therapy, including ocular therapy (i.e., for at least 6 weeks, 7 weeks, or at least 2, 3, 4, 5, or 6 months or indefinitely for the duration of the therapy) using an extended-release formulation of a compound or a pharmaceutically acceptable salt thereof of Formula I-III, including levcromakalim, in a manner that does not create significant tachyphylaxis (i.e., loss of activity over time) or tolerance, including but not limited to normal tension glaucoma. Tachyphylaxis is the decrease in response to a drug that occurs over time. It can occur after an initial dose or after a series of doses. Tolerance is the requirement to increase the dose of a drug to produce a given response.

The present invention provides a method for the use of an extended-release formulation of a compound of Formulas I, II or III or a pharmaceutically acceptable salt thereof, including levcromakalim or a salt thereof, for long-term therapy in a manner that does not induce significant tachyphylaxis or alternatively, tolerance. The loss of activity over time has been noted with a number of drugs, including for ocular therapy. For example, tachyphylaxis is a common effect of over-the-counter ocular allergy medications and is also observed using several drugs for other ophthalmic conditions, including glaucoma. Tachyphylaxis has a number of causes, including the increased or decreased expression of receptors or enzymes. This phenomenon has been noted in particular with beta adrenergic antagonists and with histamine.

The dose can be once a day or several times a day in the best judgement of the physician, and as further described herein. In one aspect, it is delivered as a topical drop for glaucoma, including normal tension glaucoma or for any form of high-pressure glaucoma, including as otherwise listed herein by example. It is advantageous to the patient to be able to take a stable dose of the drug over a lengthy period without having to change medications or dosage strength. While each patient is unique, and patients may exhibit different results based on their genetics or disease, in general, the long-term therapy using an effective amount of the extended-release formulation of a compound Formulas I, II or III or a pharmaceutically acceptable salt thereof in a suitable delivery system for the disorder to be treated is achievable according to this invention.

### Once Daily Dosing

In another embodiment, an extended release formulation is provided that is appropriate even for once-daily (QD) human dosing to treat elevated IOP glaucoma, including but not limited to primary open angle glaucoma (POAG), primary angle closure glaucoma, pediatric glaucoma, pseudo-exfoliative glaucoma, pigmentary glaucoma, traumatic glaucoma, neovascular glaucoma, iridocorneal endothelial glaucoma (primary open angle glaucoma is also known as chronic open angle glaucoma, chronic simple glaucoma and glaucoma simplex) is provided. In an alternative embodiment, once-daily (QD) human dosing is used to treat acute high-pressure glaucoma resulting from advanced cataracts. In a further alternative embodiment, once-daily (QD) human dosing is used to treat acute high-pressure glaucoma resulting from steroid induced glaucoma, uveitic glaucoma, or post-intravitreal injections. An aspect of the present invention is the ability to treat glaucoma with once-daily dosing in humans, with a controlled release formulation (for example, a gel or microparticle or nanoparticle). In a typical embodiment, it is administered with a controlled release formulation, including for example, in a simple formulation such as phosphate buffered saline or citrate buffer, optionally with an ocular excipient, including but not limited to, mannitol or another osmotic agent.

Patient compliance and adherence are serious issues, and the fewer times per day that dosing is required, the more likely compliance is achieved. Once-a-day human extended release dosing for glaucoma is advantageous to maintain the ocular pressure in the desired range to minimize optic nerve damage, while also optimizing compliance and adherence. The extended-release cromakalim formulation of a compound of Formula I, II or III or its pharmaceutically acceptable salt thereof, including levcromakalim, in the selected effective dosage in certain embodiments can be administered once a day in a topical drop or other convenient manner.

### Hyperemia

In yet another embodiment, ocular therapy using an effective amount of an extended-release formulation of a compound of Formula I, II or III or its pharmaceutically acceptable salt thereof, including levcromakalim, that does not result in significant hyperemia is provided.

Hyperemia is an excess and or prominence of blood in vessels supplying an organ. Ocular hyperemia, also called “red eye”, can include or result in vascular congestion, excessive vascular vasodilation, small bleeds, small punctate bleeds and/or micro hemorrhages. Ocular hyperemia can have a variety of causes, including but not limited to, exogenous irritants, contact lens, inflammation, vessel disruption, conjunctivitis (including infectious or allergic), trauma, endogenous ocular insults, subconjunctival hemorrhage, conjunctival hemorrhage, blepharitis, anterior uveitis, glaucoma, or irritating drugs and environmental irritants (i.e., sun and wind).

Certain ocular drugs either do not address hyperemia or actually cause hyperemia. According to the present invention, the use of an extended-release formulation of a compound of Formula I, II or III or its pharmaceutically acceptable salt thereof, including levcromakalim, does not cause significant hyperemia in the patient when used during therapy, and in one embodiment, over long-term therapy as described herein. Significant hyperemia in one embodiment is that which causes enough discoloration or discomfort to the patient that the patient considers it an adverse effect of the treatment, which can, if significant enough, lead to poor compliance and even discontinuation of therapy. The present invention can result in an advance in the art by assisting patient compliance and comfort. In one embodiment, the administration of an extended-release formulation of a compound of Formula I, Formula II, or Formula III does not significantly induce the expression of at least one protein independently selected from CD31 and VE-Cadherin.

In one embodiment, the administration of an extended-release formulation of a compound of Formula I, Formula IL, or Formula III does not significantly induce the expression of at least one protein independently selected from endothelin, fibronectin, α-SMA, phospho-eNOS, and total eNOS.

Another aspect of the present invention is the extended release treatment of glaucoma associated with Sturge Weber Syndrome, which is a congenital disorder that affects the skin, neurological system and sometimes the eyes. It is sometimes referred to as a neurocutaneous disorder. Sturge Weber Syndrome can result in Sturge Weber Syndrome-induced glaucoma, which affects 30-70% of the patients with ocular improvement. Managing Sturge Weber Syndrome-induced glaucoma can be complex, and a number of patients need surgery or a drainage device. According to the invention, Sturge Weber Syndrome-induced glaucoma can be treated by administering an effective amount of an extended-release formulation of a compound of Formulas I, II or III or a pharmaceutically acceptable salt thereof, including levcromakalim, optionally in a pharmaceutically acceptable carrier, as described herein. The patient can remain on long-term therapy under the care of a physician.

### Hypoglycemia, Hyperinsulinism, and Diabetes

Hypoglycemia is a condition caused by low levels of glucose in the blood. Glucose is the human body's main source of energy, and if the level of glucose in the blood is lower than what the body needs to support its energy demands, a number of symptoms occur. For example, the patients' blood sugar level may drop to 3.9 millimoles per liter or less. Initial symptoms of hypoglycemia include an irregular heart rhythm, fatigue, pale skin, shakiness, anxiety, sweating, hunger, irritability, a tingling sensation around the mouth, and/or crying out during sleep. As sugar levels get even lower these symptoms worsen to include confusion, visual disturbances, seizures, and a loss of consciousness. If sugar levels drop too low, death may result.

Hypoglycemia can be caused by a disorder of the endocrine system where the body no longer naturally regulates blood sugar levels appropriately. Treatment with an extended release formulation of a compound or a pharmaceutically acceptable salt thereof of Formula I-III, including levcromakalim, can help stabilize the endocrine system and thus reduce the onset or maintenance of hypoglycemia.

In one embodiment, the endocrine system abnormality causing hypoglycemia that is treated by an effective amount of an extended-release formulation of a compound or a pharmaceutically acceptable salt of Formula I-III, including levcromakalim, is hyperinsulinism. Hyperinsulinism occurs when the body has an above normal level of insulin in the blood, for example more than 175 picomoles per liter while fasting or more than 1600 picomoles per liter after eating. Insulin breaks down glucose so when its levels are too high hypoglycemia and the symptoms thereof may occur.

Diabetes is a condition in which a person's blood sugar level is too high. Diabetes is generally split into two types. Type 1 diabetes is a form of autoimmune disease which occurs when the patient's immune system attacks and destroys insulin-producing cells in the pancreas leaving the patient with little or no natural insulin. In Type 2 diabetes, the patient's cells become resistant to insulin and the pancreas is unable to make enough insulin to overcome this resistance.

Regardless of the type of diabetes, the possible symptoms include increased thirst, frequent urination, extreme hunger, unexplained weight loss, presence of ketones in urine, fatigue, irritability, blurred vision, slow-healing sores, and frequent infections.

An aspect of the present invention is the ability to administer an effective amount of an extended-release formulation of a compound or a pharmaceutically acceptable salt thereof of Formula I-III, including levcromakalim, to a patient in need thereof to treat diabetes. In one embodiment, the compound is used to treat Type 1 diabetes. In another embodiment the compound is used to treat Type 2 diabetes.

In one embodiment, the extended-release formulation of a compound or a pharmaceutically acceptable salt thereof of Formula I-III is administered in an effective amount in a parenteral dosage form for the treatment of hypoglycemia, hyperinsulinism, or diabetes. In one embodiment, the extended-release formulation of a compound of Formula I-III or pharmaceutically acceptable salt thereof is administered continuously throughout the day via an infusion and a pump. In an alternative embodiment, the extended-release formulation of a compound of Formula I-III or pharmaceutically acceptable salt thereof is administered via an oral dosage form, such as a pill, tablet, or capsule. In one embodiment, the extended-release formulation of a compound of Formula I-III or pharmaceutically acceptable salt thereof is administered at least once, twice, or three times a day.

In one embodiment, an extended-release formulation of a compound or a pharmaceutically acceptable salt thereof of Formula I-III, including levcromakalim, is administered in combination or alternation with a treatment for diabetes, including metformin, sulfonylureas (glyburide (DiaBeta, Glynase), glipizide (Glucotrol) and glimepiride (Amaryl)), meglitinides (repaglinide (Prandin) and nateglinide (Starlix)), DPP-4 inhibitors (sitagliptin (Januvia), saxagliptin (Onglyza) and linagliptin (Tradjenta)), GLP-1 receptor agonists (Exenatide (Byetta, Bydureon), liraglutide (Victoza) and semaglutide (Ozempic)), SGLT2 inhibitors (canagliflozin (Invokana), dapagliflozin (Farxiga) and empagliflozin (Jardiance)), or insulin.

### Skeletal Muscle Myopathy

Skeletal muscle myopathies (also known as myofibrillar myopathies) are disorders in which the skeletal muscle fibers contain defects that result in muscle weakness. For example, the muscle fibers may have defective sarcomeres, which are necessary for muscle contraction and are normally composed of rod-like structures called Z-disks. Z-disks link neighboring sarcomeres together to form myofibrils, the basic unit of muscle fibers. The defective sarcomeres may form clumps in the muscle fibers, significantly reducing muscle fiber strength.

An aspect of the present invention is the ability to administer an effective amount of an extended-release formulation of a compound or a pharmaceutically acceptable salt thereof of Formula I-III, including levcromakalim, to a patient in need thereof to treat a skeletal muscle myopathy. In one embodiment, an effective amount of the extended-release formulation of a compound of Formula I-III is administered parenterally, orally, or topically for the treatment of a skeletal muscle myopathy. In one embodiment, the extended-release formulation is administered intravenously. In one embodiment, the extended-release formulation is administered in combination or alternation with a corticosteroid drug (prednisone), immunosuppressant drugs (azathioprine, methotrexate, cyclosporine A, cyclophosphamide, mycophenolate mofetil, and tacrolimus), adrenocorticotropic hormone or other biological therapeutics such as rituximab or tumor necrosis factor (TNF) inhibitors (infliximab or etanercept).

In one embodiment the patient has a mutation in the desmin (DES) gene. In another embodiment, the patient has a mutation in the myotilin (MYOT) gene. In another embodiment, the patient has a mutation in the LIM-domain binding 3 (LDB3) gene. In another embodiment, the patient does not have a mutation in DES, MYOT, or LDB3.

In one embodiment, the myopathy is acquired. Acquired myopathies can be further subclassified as inflammatory myopathies, toxic myopathies, and myopathies associated with systemic conditions. In one embodiment, the inflammatory myopathy is selected from polymyositis, dermatomyositis, and inclusion body myositis (IBM). Toxic myopathies are myopathies that are drug-induced and are a side effect observed with the use of cholesterol-lowering drugs, HIV therapy, antiviral therapy, rheumatologic agents, and antifungal agents (Valiyil et al. Curr Rheumatol Rep. 2010, 12, 213). Therefore, in one embodiment, an effective amount of an extended-release formulation of a compound or a pharmaceutically acceptable salt thereof of Formula I-III, including levcromakalim, is administered for the treatment of a toxic myopathy induced by a medication. Non-limiting examples of medications that induce toxic myopathy include steroids, cholesterol-lowering medications (for example, statins, fibrates, niacin, and ezetimibe), propofol, amiodarone, colchicine, chloroquine, antivirals and protease inhibitors, omeprazole, and tryptophan.

In an alternative embodiment, an effective amount of an extended-release formulation of a compound or a pharmaceutically acceptable salt thereof of Formula I-III, including levcromakalim, is administered for the treatment of a myopathy associated with systemic conditions. Non-limiting examples of systemic diseases include endocrine disorders, systemic inflammatory diseases, electrolyte imbalance, critical illness myopathy, and amyloid myopathy.

In one embodiment, the myopathy is inherited. Inherited myopathies can be further subclassified as muscular dystrophies, congenital myopathies, mitochondrial myopathies, and metabolic myopathies. In one embodiment, an effective amount of an extended-release formulation of a compound or a pharmaceutically acceptable salt thereof of Formula I-III, including levcromakalim, is administered for the treatment of muscular dystrophy, including dystrophinopathy (Duchenne muscular dystrophy), myotonic dystrophy 1 and 2, facioscapulohumeral muscular dystrophy, oculopharyngeal muscular dystrophy, or limb girdle muscular dystrophy. In one embodiment, an effective amount of an extended-release formulation of a compound or a pharmaceutically acceptable salt thereof of Formula I-III, including levcromakalim, is administered for the treatment of congenital myopathy, including nemaline myopathy or central core myopathy. In one embodiment, an effective amount of an extended-release formulation of a compound or a pharmaceutically acceptable salt thereof of Formula I-III, including levcromakalim, is administered for the treatment of a metabolic myopathy, including acid maltase or acid alpha-1,4-glucosidase deficiency (Pompe's disease), glycogen storage disorders 3-11, carnitine deficiency, fatty acid oxidation defects, or carnitine palmitoyl transferase deficiency. In one embodiment, an effective amount of an extended-release formulation of a compound or a pharmaceutically acceptable salt thereof of Formula I-III, including levcromakalim, is administered for the treatment of a mitochondrial myopathy, including Kearns-Sayre syndrome (KSS), mitochondrial DNA depletion syndrome (MDS), mitochondrial encephalomyopathy lactic acidosis and stroke-like episodes (MELAS), maternally inherited deafness and diabetes (MIDD), mitochondrial neurogastrointestinal encephalomyopathy (MNGIE), myoclonus epilepsy with ragged red fibers (MERRF), neuropathy ataxia, and retinitis pigmentosa (NARP), or Pearson syndrome.

### Erectile Dysfunction and Female Sexual Arousal Disorder Due to Blood Flow

Erectile dysfunction is a disorder characterized by a persistent difficulty having and/or maintaining an erection. Erectile dysfunction can be caused by a variety of factors including psychological, emotional, and physical problems. An aspect of the present invention is the administration of an effective amount of an extended-release formulation of a compound or its pharmaceutically acceptable salt of Formula I-III, including levcromakalim, to a patient in need thereof to treat erectile dysfunction. In one embodiment, the patient with erectile dysfunction has low blood flow to their pubic area. Therefore, in one aspect an extended-release formulation of a compound or a pharmaceutically acceptable salt thereof of Formula I-III, including levcromakalim, or a pharmaceutically acceptable salt thereof increases blood flow to the pubic area.

Female sexual arousal disorder is a disorder characterized by a persistent difficulty having and/or maintaining sexual arousal. Female sexual arousal disorder can be caused by a variety of factors including psychological, emotional, and physical problems. An aspect of the present invention is the ability to administer an effective amount of an extended-release formulation of a compound or a pharmaceutically acceptable salt thereof of Formula I-III, including levcromakalim, to a patient in need thereof to treat Female sexual arousal disorder. In one embodiment, the patient with Female sexual arousal disorder has low blood flow to her pubic area. Therefore, in one embodiment, the extended-release formulation of a compound or a pharmaceutically acceptable salt thereof of Formula I-III, including levcromakalim, increases blood flow to the pubic area.

In one embodiment, the extended-release formulation of a compound of Formula I-III is administered in an effective amount orally as needed for the treatment of erectile dysfunction or Female sexual arousal disorder. In one embodiment, the extended-release formulation can be administered topically in an effective amount as a cream, gel, or ointment, taken as needed, for the treatment of erectile dysfunction or Female sexual arousal disorder. In certain embodiments the extended-release formulation of a compound of Formula I-III, for example cromakalim, is formulated as an active agent in a lubricant for treatment of erectile dysfunction and/or Female sexual arousal disorder.

In certain embodiments, the extended-release formulation of a compound of Formula I-III or its pharmaceutically acceptable salt thereof, including levcromakalim, is administered in an effective amount in combination or alternation with one or more additional treatments for erectile dysfunction, including but not limited to a phosphodiesterase inhibitor (for example, sildenafil, dildenafil citrate, vardenafil, vardenafil HCl, tadalafil, avanafil), testosterone therapy, a penile injection (for example, ICI or intracavernosal alprostadil), intraurethral medication (for example, IU or alprostadil), penile implants, a combination of therapies (for example, bimix or trimix) or surgery.

In certain embodiments, an effective amount of the extended-release formulation of a compound of Formula I-III or its pharmaceutically acceptable salt, for example cromakalim, is administered in combination with one or more additional treatments for Female sexual arousal disorder, including but not limited to estrogen therapy, an estrogen receptor modulator (for example, ospemifene), androgen therapy, an antidepressant (for example, flibanserin), or melanocortin agonist (for example, bremelanotide).

### Hypotrichosis and Baldness

Hypotrichosis of the eyebrows and eyelashes is a disorder in which there is little to no growth of hair, or an insufficient amount of hair, on the eyebrows and/or eyelashes at the edge of eyelids.

An aspect of the present invention is the ability to administer an effective amount of an extended-release formulation of a compound or a pharmaceutically acceptable salt thereof of Formula I-III, including levcromakalim, to a patient in need thereof to treat hypotrichosis. In one embodiment the patient has a genetic mutation that causes hypotrichosis. In another embodiment the patient does not have a genetic mutation that causes hypotrichosis.

In one embodiment, the extended-release formulation of a compound of Formula I-III is administered as a topical dosage form applied to the upper eyelid margin at the base of the eyelashes. In one embodiment, the extended-release formulation of a compound of Formula I-III is administered at least once a day or twice a day.

In certain embodiments, the compound of the present invention is provided in an effective amount in combination or alternation with a prostaglandin analog (for example, bimatoprost).

Baldness is hair loss or the absence of hair, most typically on the scalp. Common types of baldness include male or female pattern baldness, alopecia areata, telogen effluvium (the loss of hair after a stressful situation), and anagen effluvium (abnormal hair loss during the first phase of the hair growth cycle). In one embodiment, an effective amount of an extended-release formulation of a compound or a pharmaceutically acceptable salt thereof of Formula I-III, including levcromakalim, is administered to a patient in need thereof to treat baldness. In one embodiment, the baldness is male or female pattern baldness. In one embodiment, the baldness is alopecia areata. In one embodiment, the baldness is telogen effluvium. In one embodiment, the baldness is anagen effluvium.

### Neuropathic Pain and Neurodegenerative Diseases (for Example Parkinson's Disease and Huntington's Disease)

Neuropathic pain is a disorder in which nerve damage, or a malfunctioning nervous system causes shooting or burning pain. Neuropathic pain can be acute or chronic and may be caused by a variety of factors including alcoholism, amputation, chemotherapy, diabetes, facial nerve problems, AIDS, multiple myeloma, multiple sclerosis, nerve or spinal cord compression, herniated disk, arthritis, shingles, spine surgery, syphilis, or thyroid problems. Patients with neuropathic pain may experience a shooting and burning pain or a tingling or numbness sensation.

An aspect of the present invention is the ability to administer an effective amount of an extended-release formulation of a compound or a pharmaceutically acceptable salt thereof of Formula I-II, including levcromakalim, to a patient in need thereof to treat neuropathic pain.

In one embodiment, an effective amount of the extended-release formulation of a compound of Formula I-III or its pharmaceutically acceptable salt is administered orally, enterally, or parenterally for the treatment of neuropathic pain. The extended-release formulation can be administered once, twice, or three times a day according to the instructions of the healthcare provider, for as long as necessary.

In one embodiment, for the treatment of neuropathic pain, an effective amount of an extended-release formulation of a compound of Formula I-III or its pharmaceutically acceptable salt is used in combination or alternation with a calcium channel α2-delta ligand (for example, pregabalin or gabapentin), a tricyclic antidepressant (for example, amitriptyline, nortriptyline, or desipramine), an SNRI antidepressant (for example, duloxetine or venlafaxine), or an opioid (for example, tramadol or tapentadol).

Neurodegenerative diseases are those that cause or result from the degeneration of the patient's nerves. This cellular process includes a neuroinflammatory reaction that involves the activation of glial cells, including microglia and astrocytes. A neurodegenerative disease may make it difficult for the patient to balance, move, talk, breathe, or remember. Neurodegenerative diseases include amyotrophic lateral sclerosis (ALS), Fredreich's ataxia, Huntington's disease, Lewy body disease, Parkinson's disease, and spinal muscular atrophy.

An aspect of the present invention is the administration of an effective amount of an extended-release formulation of a compound of the present invention, for example cromakalim, or its pharmaceutically acceptable salt to a patient in need thereof to treat a neurodegenerative disease. In one embodiment the neurodegenerative disease is Parkinson's disease. In another embodiment the neurodegenerative disease is Huntington's disease. In an alternative embodiment the neurodegenerative disease is Alzheimer's disease.

The therapy for a neurodegenerative disease includes combination or alternation therapy with an effective amount of a compound as disclosed herein. Drugs for Parkinson's disease include amantadine, nilotinib, zonisamide, selegiline, methylphenidate, and salbutamol. Drugs for Huntington's disease include tetrabenazine, tiapride, clozapine, olanzapine, risperidone, quetiapine, and memantine. Drugs for amyotrophic lateral sclerosis (ALS) include mastinib, dolutegravir, abacavir, lamivudine, retigabine, and tamoxifen. Drugs for Lewy body disease include donepezil, galantamine, and rivastigmine. Drugs for spinal muscular atrophy include Nusinersen and Onasemnogene abeparvovec.

Following ischemia, stroke, convulsions, or trauma, neuroprotective drugs are often administered to prevent damage to the brain and/or spinal cord. In one embodiment, an effective amount of an extended-release formulation of a compound of Formula I-III or its pharmaceutically acceptable salt thereof, including levcromakalim, is administered as a neuroprotective agent. In one embodiment, the extended-release compound is administered following ischemia, stroke, convulsions, or trauma. In one embodiment, an effective amount of an extended-release formulation of a compound of Formula I-III or its pharmaceutically acceptable salt thereof, including levcromakalim, is administered as a cellular protective agent.

### Tumor Hypoperfusion and Hypoxia

In one aspect an effective amount of an extended-release formulation of a compound or a pharmaceutically acceptable salt of Formula I-III, including levcromakalim, is administered to a patient to treat tumor hypoperfusion or tumor hypoxia. Tumor hypoperfusion refers to reduced blood flow in the tumor. Tumor hypoxia refers to a reduced level of oxygen in the tumor cells.

There can be overlap between the two.

When a tumor is in a state of hypoperfusion, perhaps because it is growing quickly, it does not have sufficient blood flow to allow tumor therapeutics to have access to the tumor cells. This can create resistance to chemotherapeutic treatment. In one embodiment, an extended-release formulation of a compound or a pharmaceutically acceptable salt thereof of Formula I-III, including levcromakalim, is administered to a patient with hypoperfusion of a tumor so that the tumor is more easily treated with anti-tumor medication such as chemotherapy.

In another embodiment, the extended-release formulation of a compound or a pharmaceutically acceptable salt of Formula I-III, including levcromakalim, is administered to a patient with hypoperfusion of non-tumor cells, for example as a result of trauma.

When a tumor is hypoxic, it is in a low oxygen state due to the lack of oxygen in the cell.

Tumors that are hypoxic can be more likely to exhibit metastatic behavior. Therefore, in one aspect, an extended-release formulation of a compound or a pharmaceutically acceptable salt thereof of Formula I-III, including levcromakalim, is administered in an effective amount to a patient to treat tumor hypoxia, optionally in combination or alternation with chemotherapy or other anti-tumor treatment.

In another embodiment, an effective amount of an extended-release formulation of the compound of the present invention or its pharmaceutically acceptable salt is administered to treat hypoxia or hypoperfusion optionally in combination with a vascular endothelial growth factor (VEFG) therapy.

In an alternative embodiment, an effective amount of an extended-release formulation of a compound of Formula I-III or its pharmaceutically acceptable salt is used in combination or alternation with oxygen therapy (for example, an oxygen mask or a small tube clipped under the nose to provide supplemental oxygen) or an asthma medication (for example, fluticasone, budesonide, mometasone, beclomethasone, ciclesonide, montelukast, zafirlukast, zileuton, salmeterol, formoterol, vilanterol, albuterol, levalbuterol, prednisone, methylprednisone, omalizumab, mepolizumab, benralizumab, or resilzumab).

### Selected Cardiovascular Disorders

Unstable angina is a condition in which the heart does not get enough blood and oxygen from the narrowing of coronary arteries, causing unexpected chest pain and discomfort. The most common cause of the condition is coronary artery disease due to atherosclerosis. Angina can be treated with angioplasty and stent placement or enhanced external counterpulsation. Several medications can also improve symptoms, and these include aspirin, nitrates, beta blockers, statins, and calcium channel blockers. Many of these drugs have unwanted side effects. In one embodiment, an effective amount of an extended-release formulation of a compound or a pharmaceutically acceptable salt thereof of Formula I-III, including levcromakalim, is administered to a patient with unstable angina and the associated chest pains.

In one embodiment, an effective amount of an extended-release formulation of a compound or a pharmaceutically acceptable salt thereof of Formula I-III, for example cromakalim, is administered in combination with angioplasty, stent placement, and/or enhanced external counterpulsation. In another embodiment, an extended-release formulation of a compound or a pharmaceutically acceptable salt thereof of Formula I-III, for example cromakalim, is administered in combination or alternation with aspirin, nitrate, a beta blocker, a statin, or a calcium channel blocker.

Congestive heart failure (CHF) is a chronic progressive condition in which the ventricles of the heart are not capable of pumping enough blood volume to the rest of the body. The most typical form of CHF is left-sided CHF where the left ventricle does not properly pump blood, and this often progresses to the right-side. The four stages of CHF are indicative of the severity of the disease and also determine various treatment options. If left untreated, blood and other fluids can back up inside the lungs, abdomen, liver, and the lower body and can be life-threatening.

Medications for CHF include ACE inhibitors, beta-blockers, and diuretics. Each of these medications have associated side effects. For example, ACE inhibitors have the potential to raise potassium levels in the blood and cannot be tolerated in some patients. For this reason, in one embodiment, an effective amount of an extended-release formulation of a compound or a pharmaceutically acceptable salt thereof of Formula I-III, including levcromakalim, is administered to a patient with CHF. The heart failure may be in Stage 1, Stage 2, Stage 3, or Stage 4.

Chronic or acute myocardial ischemia is the inability of blood flow to reach the heart, which prevents the heart from receiving enough oxygen. Myocardial ischemia can be caused by atherosclerosis, a blood clot, or a coronary artery spasm. Myocardial ischemia can cause serious abnormal heart rhythms or even lead to a heart attack. Current treatment for myocardial ischemia may include the administration of an aspirin, nitrate, beta-blocker, ACE inhibitor, or cholesterol-lowering medication, each of which has side effects and efficacies of various degrees. In one embodiment, an effective amount of an extended-release formulation of a compound or a pharmaceutically acceptable salt of Formula I-III, including levcromakalim, is administered to a patient with chronic or acute myocardial ischemia.

Microvascular dysfunction (or coronary microvascular disease) is a type of non-obstructive coronary artery disease that causes the small blood vessels feeding the heart muscle to not work.

Patients with microvascular dysfunction do not have plaque buildup in the coronary artery blood vessels, but have damage to the inner walls of the blood vessels that can lead to spasms and decrease blood flow to the heart muscle. In an alternative embodiment of the invention, a an extended-release formulation of a compound or a pharmaceutically acceptable salt of Formula I-III, including levcromakalim, is provided in an effective amount for the treatment of microvascular dysfunction.

Coronary artery disease is the buildup of plaque in the walls of coronary arteries, vessels that supply the heart with blood. This plaque narrows the arteries, slowing blood flow, and if a piece of plaque breaks off and lodges in an artery, it can block blood flow completely. The blockage of blood flow to the heart by a plaque and/or blood clot is referred to as acute myocardial infarction, often referred to as a heart attack. Symptoms vary, but often include pressure or tightness in the chest and arms, shortness of breath, and/or sudden dizziness. Emergency medical assistance is typically required. The patient may be administered one or a variety of drugs, including aspirin, a thrombolytic, an antiplatelet agent, a blood-thinning medication, a nitroglycerin, a beta blocker, ACE inhibitor, or statin. Potential surgical procedures include angioplasty or bypass surgery. Following a heart attack, cardiac rehabilitation is required that includes medication to prevent another heart attack and subsequent complications.

Given the life-threatening nature of a heart attack, it is advantageous to have a number of potential therapeutic agents as possible treatment options. Therefore, in one embodiment, an effective amount of an extended-release formulation of a compound or a pharmaceutically acceptable salt of Formula I-III, including levcromakalim, is administered to a patient experiencing a heart attack and/or as a therapy in cardiac rehabilitation. The drug is administered for a time period determined by the health care provider, including but not limited to at least two weeks, one month, two months, three months, or more. In one embodiment, an extended-release formulation of a compound or a pharmaceutically acceptable salt of Formula I-III, including levcromakalim, acts as a cardioprotective agent during the heart attack. In one embodiment, an effective amount of an extended-release formulation of a compound or a pharmaceutically acceptable salt of Formula I-II, including levcromakalim, is used as a cardioprotective agent in a host undergoing heart surgery. In one embodiment, the host is undergoing a cauterization procedure. In one embodiment, an effective amount of an extended-release formulation of a compound or a pharmaceutically acceptable salt thereof of Formula I-III, including levcromakalim, is administered for the treatment of left ventricular failure after an acute myocardial infarction (AMI) or heart attack. In an alternative embodiment of the present invention, an effective amount of an extended-release formulation of a compound or a pharmaceutically acceptable salt thereof of Formula I-III, including levcromakalim, is administered for the treatment of coronary artery disease.

In one embodiment, an effective amount of an extended-release formulation of a compound or a pharmaceutically acceptable salt thereof of Formula I-III, for example cromakalim, is administered in combination or alternation with an ACE inhibitor, beta-blocker, aspirin, nitrate, a cholesterol-lowering medication, statin, or diuretic.

In one embodiment, an extended-release formulation of a compound or a pharmaceutically acceptable salt of Formula I-III, including levcromakalim, is provided in an effective amount for the preservation of the heart prior to organ donation.

Arrhythmia is the improper (too fast, too slow, or irregular) beating of the heart, which can be caused by a variety of medical conditions, including coronary artery disease, high blood pressure, electrolyte imbalances, or injury from a heart attack. Arrhythmia is very common, affecting 3 million people in the US every year. The majority of arrhythmia may be harmless, however very abnormal arrhythmia can cause serious or fatal symptoms. If left untreated, arrhythmia can affect the heart, the brain, and other organs because not enough blood is able to reach the organs. Implantable devices for the treatment of arrhythmias include a pacemaker or an implantable cardioverter-defibrillator (ICD). In one embodiment, an extended-release formulation of a compound or a pharmaceutically acceptable salt of Formula I-III, including levcromakalim, is administered to a patient with arrhythmia. In one embodiment, an extended-release formulation of a compound or a pharmaceutically acceptable salt of Formula I-III, including levcromakalim, is provided in an amount effective to treat or prevent arrhythmias and/or ventricular fibrillation associated with AMI in a host in need thereof.

In one embodiment, an extended-release formulation of a compound or a pharmaceutically acceptable salt of Formula I-III, for example cromakalim, is administered in combination with a pacemaker or ICD.

The endothelial layer is a layer of cells lining all blood vessels and is responsible for proper dilation and constriction of blood vessels. Endothelial tone is the balance between constriction and dilation and largely determines a person's blood pressure. Endothelial dysfunction is the failure of the endothelial layer to regulate dilation/constriction. Endothelial dysfunction is a well-established response to cardiovascular risk factors and in turn, often precedes the development of atherosclerosis. Treatments include ACE inhibitors and statin drugs, but studies for additional drugs are underway. In one embodiment, an effective amount of an extended-release formulation of a compound or a pharmaceutically acceptable salt thereof of Formula I-III, including levcromakalim, is administered to a patient with endothelial dysfunction.

A transient ischemic attack (TIA) is similar to a stroke, but only lasts a few minutes and leaves no permanent damage. Like a stroke, a clot in the blood supply travels to the brain. The signs of a TIA include weakness, numbness, paralysis, slurred speech, dizziness, blindness, and/or a sudden, severe headache. Following a diagnosis of a TIA, it is important to try to prevent another TIA or a stroke. Typical medications include anti-platelet drugs, anticoagulants, and thrombolytic agents. Alternatively, angioplasty is often recommended. Anti-platelet drugs and anticoagulants have to be taken with caution since they increase the risk of bleeding. For this reason, vasodilators represent an alternative medication for TIA. In one embodiment, an effective amount of an extended-release formulation of a compound or a pharmaceutically acceptable salt of Formula I-III, including levcromakalim, is administered to a patient diagnosed with a transient ischemic attack.

Carotid artery disease is the buildup of plaque in the carotid arteries that run along either side of the neck and supply blood to the brain, face, and neck. If a piece of plaque breaks off and causes a clot in a blood vessel leading to the brain, the clot can cause a stroke. In an alternative embodiment of the invention, an effective amount of an extended-release formulation of a compound or a pharmaceutically acceptable salt of Formula I-III, including levcromakalim, is administered to a patient diagnosed with a stoke.

In one embodiment, an extended-release formulation of a compound or a pharmaceutically acceptable salt of Formula I-III, for example cromakalim, is administered in combination or alternation with an anti-platelet drug, anticoagulant, or thrombolytic agent.

High blood pressure is a condition where the force of blood flowing through the blood vessels is consistently high. This can often lead to many conditions, including heart conditions discussed herein and stroke. In an alternative embodiment of the invention, an effective amount of an extended-release formulation of a compound or a pharmaceutically acceptable salt of Formula I-III, including levcromakalim, is administered to a patient with high blood pressure as a treatment to lower blood pressure.

Blood Vessel Disorders Raynaud's disease is a rare disorder of blood vessels in which fingers and toes feel numb in response to cold temperature or stress. This can induce a color change (usually white and then blue) of fingers and toes accompanied by a feeling of numbness. This is caused by arteries in fingers and toes undergoing vasospasms when exposed to cold or stress and this then narrows vessels and temporarily limits blood supply. In one embodiment an effective amount of an extended-release formulation of a compound or a pharmaceutically acceptable salt of Formula I-III, including levcromakalim, is administered to a patient for the treatment of Raynaud's disease, which may be via topical, enteric or parenteral delivery.

Peripheral artery disease (PAD) is a disease in which plaque builds up in arteries that carry blood to limbs, the heart, and other organs. This causes narrowed arteries that reduce blood flow from the heart. PAD can cause an embolism or thrombosis, which can lead to acute limb disease.

Acute limb disease is treatable, but if left untreated (a delay of 6-12 hours), it can result in amputation and/or death. Symptoms include pain, pallor, and/or paralysis. In one embodiment, an effective amount of an extended-release formulation of a compound or a pharmaceutically acceptable salt of Formula I-III, including levcromakalim, is administered for the treatment of acute limb ischemia.

Chronic limb ischemia is a type of advanced PAD that develops over time and includes muscular pain, patellofemoral pain, and eventual tissue loss due to poor perfusion and hypoxia.

Chronic limb ischemia is associated with diabetes, smoking, and high blood pressure. In one embodiment, an effective amount of an extended-release formulation of a compound or a pharmaceutically acceptable salt of Formula I-III, including levcromakalim, is administered for the treatment of chronic limb ischemia.

Thrombophlebitis is when a blood clot forms in a vein and slows down the blood flow in the vein. It most often affects legs but can also happen in arms or other veins in the body.

Thrombophlebitis can happen right under the skin or deeper in legs or arms. Types of thrombophlebitis include superficial phlebitis or superficial thrombophlebitis that occur just below the surface of the skin; deep vein thrombosis (DVT) that occurs deep in the body; and, migratory thrombophlebitis (Trousseau's syndrome or thrombophlebitis migrans), which is when a clot comes back in a different part of the body. In an alternative embodiment of the invention, an effective amount of an extended-release formulation of a compound or a pharmaceutically acceptable salt of Formula I-III, including levcromakalim, is administered for the treatment of thrombophlebitis. In one embodiment, the thrombophlebitis is superficial thrombophlebitis. In one embodiment, the thrombophlebitis is deep vein thrombosis. In one embodiment, the thrombophlebitis is migratory thrombophlebitis.

Chronic venous insufficiency (CVI) is a condition that occurs when the venous wall and/or valves in the leg veins are not working effectively, making it difficult for blood to return to the heart from the legs. CVI causes blood to “pool” or collect in these veins, and this pooling is called stasis. If CVI is not treated, the pressure and swelling increases until the tiniest blood vessels in the legs (capillaries) burst. When this happens, the overlying skin takes on a reddish-brown color and is very sensitive to being broken if bumped or scratched. In an alternative embodiment of the invention, an effective amount of an extended-release formulation of a compound or a pharmaceutically acceptable salt of Formula I-III, including levcromakalim, is administered for the treatment of chronic venous insufficiency.

Pulmonary arterial hypertension (PAH) is a rare disease that usually presents in young adulthood, predominantly in women. PAH is a progressive disorder of the pulmonary arteries leading to the lungs and is fatal despite currently available therapies. In one embodiment an effective amount of an extended-release formulation of a compound or a pharmaceutically acceptable salt of Formula I-III, including levcromakalim, is administered to a patient for the treatment of pulmonary arterial hypertension. In one embodiment, an extended-release formulation of a compound or a pharmaceutically acceptable salt of Formula I-III is administered in combination with a PDE-5 inhibitor (for example, sildenafil or tadalafil), a prostanoid vasodilators (for example, epoprostenol, treprostinil, or iloprost), a guanylate cyclase stimulators (for example, riociguat), or an endothelin receptor antagonist (for example, bosentan, ambrisentan, or macitentan).

Aspects of the invention include administering the drug as described herein in combination or alternation with a calcium channel blocker (for example nifedipine, afeditab, Procardia, amlodipine, felodipine, bepridil, diltiazem, nicardipine, nisoldipine, verapamil and isradipine) or another vasodilator (for example hydralazine, nitroglycerin, alprostadil, riociguat, nesiritide, nitroprusside, sildenafil, and minoxidil).

### Lymphatic Diseases

The lymphatic system acts to rid the bodies of toxins and waste and its primary role is to transport lymph, a fluid containing white blood cells, throughout the body to fight infection. The system is primarily composed of lymphatic vessels that are connected to lymph nodes, which filter lymph. K_{ATP} channels are expressed by lymphatic muscle cells and studies have shown that certain K_{ATP} channel openers dilate lymphatic vessels.

For example, as discussed in a recent study by Garner et al. (“KATP Channel Openers Inhibit Lymphatic Contractions and Lymph Flow as a Possible Mechanism of Peripheral Edema”, Journal of Pharmacology and Experimental Therapeutics, Oct. 25, 2020) rhythmic contractions of isolated rat mesenteric lymph vessels are progressively impaired when exposed to K_{ATP} channel openers, such as cromakalim, minoxidil sulfate, and diazoxide. Increasing concentrations of cromakalim ultimately abolished the contractions of the vessels and impaired flow through the vessels by attenuating the frequency and amplitude of the contractions. Similar effects were observed with minoxidil sulfate and diazoxide when administered at clinically relevant concentrations.

Inflammation of the lymph vessels is known as lymphangitis and symptoms generally include swelling, redness, and/or pain in the infected area. In one embodiment, an effective amount of an extended-release formulation of a compound or a pharmaceutically acceptable salt of Formula I-II, including levcromakalim, is administered for the treatment of lymphangitis.

The lymph nodes can also become infected with a virus, bacteria, and/or fungi and this is referred to as lymphadenitis. Symptoms of lymphadenitis also include redness or swelling around the lymph nodes. In one embodiment, an effective amount of an extended-release formulation of a compound or a pharmaceutically acceptable salt of Formula I-III, including levcromakalim, is administered for the treatment of lymphangitis, and in one embodiment, the extended-release formulation of a compound of Formula I-Formula III is administered in combination with an antibiotic or antifungal medication.

A common cancer of the lymph system is Hodgkin's lymphoma, in which cancer originates from the white blood cells called lymphocytes. The cancer can begin in any part of the body and symptoms include non-painful enlarged lymph nodes in the neck, under the arm, or in the groin.

There are two major types of Hodgkin lymphoma: classical Hodgkin lymphoma and nodular lymphocyte-predominant Hodgkin lymphoma. Treatment for Hodgkin's lymphoma includes chemotherapy and/or radiation, and the most common treatment is the monoclonal antibody rituximab (Rituxan). In one embodiment, an effective amount of an extended-release formulation of a compound or a pharmaceutically acceptable salt of Formula I-III, including levcromakalim, is administered for the treatment of Hodgkin's lymphoma, in combination with chemotherapy and/or radiation. In one embodiment, the chemotherapy is rituximab.

Non-Hodgkin's lymphoma is caused when the body produces too many abnormal white blood cells called lymphocytes, which leads to tumors. A common subtype of non-Hodgkin's lymphoma is B-Cell Non-Hodgkin's lymphoma. Symptoms include swollen lymph nodes, fever, and/or chest pain. Non-Hodgkin's lymphoma is treated with chemotherapy and/or radiation. A common treatment is a regimen known as R-CHOP that consists of cyclophosphamide, doxorubicin, vincristine, and prednisone, plus the monoclonal antibody rituximab (Rituxan). In one embodiment, an effective amount of an extended-release formulation of a compound or a pharmaceutically acceptable salt of Formula I-III, including levcromakalim, is administered for the treatment of non-Hodgkin's lymphoma, in combination with chemotherapy and/or radiation. In one embodiment, the chemotherapy consists of cyclophosphamide, doxorubicin, vincristine, prednisone, and rituximab.

Castleman's disease is a group of lymphoproliferative disorders characterized by lymph node enlargement and there are at least three distinct subtypes: unicentric Castleman disease (UCD), human herpesvirus 8 associated multicentric Castleman disease (HHV-8-associated MCD), and idiopathic multicentric Castleman disease (iMCD). In UCD, enlarged lymph nodes are present in a single region and in iMCD, enlarged lymph nodes are present in multiple regions. HHV-8-Associated MCD is similar to iMCD in that enlarged lymph nodes are present in multiple regions, but the patient is also infected with human herpesvirus 8.

In one embodiment, an effective amount of an extended-release formulation of a compound of Formula I-Formula III or its pharmaceutically acceptable salt thereof, including levcromakalim, is administered for the treatment of Castleman's disease, including unicentric Castleman disease (UCD), human herpesvirus 8 associated multicentric Castleman disease (HHV-8-associated MCD), and idiopathic multicentric Castleman disease (iMCD).

Lymphangiomatosis is a disease where cysts and/or lesions are formed from lymphatic vessels. The masses are not present in one single localized mass, but are widespread. It is a multi-system disorder where abnormally proliferating lymphatic channels expand and infiltrate surrounding tissues, bones, and organs. It is a rare disease that is most widespread in children and teenagers. There is no standard treatment and often treatments are only aimed at reducing symptoms. In one embodiment, an effective amount of an extended-release formulation of a compound of Formula I-Formula III or its pharmaceutically acceptable salt is administered for the treatment, or the reduction of symptoms associated with lymphangiomatosis.

Lymphangiectasia, also known as “lymphangiectasis”, is a pathologic dilation of lymph vessels. When it occurs in the intestines, it causes a disease known as “intestinal lymphangiectasia” that is characterized by lymphatic vessel dilation, chronic diarrhea, and loss of proteins such as serum albumin and globulin. In one embodiment, an effective amount of an extended-release formulation of a compound of Formula I-Formula III or its pharmaceutically acceptable salt thereof, including levcromakalim, is administered for the treatment or the reduction of symptoms associated with lymphangiectasia.

The eye is unique in that certain parts of the eye are lymphatic rich, while other parts of the eye of devoid of lymphatics. Parts of the eye, including the eyelids, lacrimal glands, conjunctiva, limbus, optic nerve sheath, extraocular muscles, connective tissues of the extraocular muscle cones, are lymphatic rich, while the cornea and retina are lymphatic-free. A number of lymphatic disorders have been identified in the eye. Ocular lymphatic disorders include, but are not limited to, conjunctival myxoma, dry eye, conjunctival lymphangiectasia, chemosis, mustard gas keratitis, corneal inflammation, orbital cellulitis, chalazion, dermatochalasis, and blepharochalasis. In one embodiment, an effective amount of an extended-release formulation of a compound of Formula I-Formula III or its pharmaceutically acceptable salt thereof, including levcromakalim, is administered for the treatment of an ocular lymphatic disorders. In one embodiment, the ocular lymphatic disorder is selected from conjunctival myxoma, dry eye, conjunctival lymphangiectasia, chemosis, mustard gas keratitis, corneal inflammation, orbital cellulitis, chalazion, dermatochalasis, and blepharochalasis.

There is also evidence that lymphatic vessels, but not angiogenic vessels, are important for immune rejection after corneal transplantation (T. Dietrich et al., Journal of Immunology, 2010, 184, 2, 535-539). Therefore, in one embodiment, an extended-release formulation of a compound of Formula I-Formula III or its pharmaceutically acceptable salt thereof, including levcromakalim, is administered following a corneal transplant to reduce the risk of immune rejection.

### Mitochondrial Disorders

Mitochondrial diseases are long-term, genetic, and often inherited. The diseases are a clinically heterogeneous group of disorders that result from a dysfunction in the mitochondrial respiratory chain. The mitochondrial respiratory chain is the essential final common pathway for aerobic metabolism, and tissues and organs that are highly dependent on aerobic metabolism are preferentially involved in mitochondrial disorders. While some mitochondrial disorders only affect a single organ, many involve multiple organ systems and often present with prominent neurologic and myopathic features. Mitochondria contain a potassium specific channel (mitoKATP channel) sensitive to ATP. The mitochondrial KATP channel plays an important role in the mitochondrial volume control and in regulation of the components of protonmotive force.

Mitochondria are unique in that they have their own DNA called mitochondrial DNA, or mtDNA. Mutations in this mtDNA or mutations in nuclear DNA (DNA found in the nucleus of a cell) can cause mitochondrial disorder. Environmental toxins can also trigger mitochondrial disease. In one embodiment, an effective amount of an extended-release formulation of a compound of Formula I-Formula III or its pharmaceutically acceptable salt thereof, including levcromakalim, is administered for the treatment of a mitochondrial disorder.

Inside the mitochondrion is a group of proteins that carry electrons along four chain reactions (Complexes I-IV), resulting in energy production. This chain is known as the Electron Transport Chain. A fifth group (Complex V) churns out the ATP. Together, the electron transport chain and the ATP synthase form the respiratory chain and the process is known as oxidative phosphorylation or OXPHOS. Complex I, the first step in this chain, is the most common site for mitochondrial abnormalities, representing as much as one third of the respiratory chain deficiencies. Often presenting at birth or in early childhood, a Complex I deficiency is usually a progressive neuro-degenerative disorder and is responsible for a variety of clinical symptoms, particularly in organs and tissues that require high energy levels, such as brain, heart, liver, and skeletal muscles. In one embodiment, an effective amount of an extended-release formulation of a compound of Formula I-Formula III or its pharmaceutically acceptable salt thereof, including levcromakalim, is administered for the treatment of a Complex I deficiency.

A number of specific mitochondrial disorders have been associated with Complex I deficiency including Leber's hereditary optic neuropathy, mitochondrial encephalomyopathy lactic acidosis and stroke-like episodes (MELAS), myoclonic epilepsy with ragged red fibers (MERRF), and Leigh Syndrome.

Mitochondrial encephalomyopathy lactic acidosis and stroke-like episodes (MELAS) is a progressive neurodegenerative disorder with typical onset between the ages of two and fifteen, although it may occur in infancy or as late as adulthood. Initial symptoms may include stroke-like episodes, seizures, migraine headaches, and recurrent vomiting. The stroke-like episodes, often accompanied by seizures, are the hallmark symptom of MELAS and cause partial paralysis, loss of vision, and focal neurological defects. The gradual cumulative effects of these episodes often result in the variable combinations of loss of motor skills (speech, movement, and eating), impaired sensation (vision loss and loss of body sensations), and mental impairment (dementia). MELAS patients may also suffer additional symptoms including muscle weakness, peripheral nerve dysfunction, diabetes, hearing loss, cardiac and kidney problems, and digestive abnormalities. Lactic acid usually accumulates at high levels in the blood, cerebrospinal fluid, or both. In one embodiment, an effective amount of an extended-release formulation of a compound of Formula I-Formula III or its pharmaceutically acceptable salt thereof, including levcromakalim, is administered for the treatment of mitochondrial encephalomyopathy lactic acidosis and stroke-like episodes (MELAS).

Myoclonic epilepsy with ragged red fibers (MERRF) is a multisystem disorder characterized by myoclonus, which is often the first symptom, followed by generalized epilepsy, ataxia, weakness, and dementia. Symptoms usually first appear in childhood or adolescence after normal early development. In over 80% of cases, MERRF is caused by mutations in the mitochondrial gene called MT-TK. In one embodiment, an effective amount of an extended-release formulation of a compound of Formula I-Formula III or its pharmaceutically acceptable salt thereof, including levcromakalim, is administered for the treatment of myoclonic epilepsy with ragged red fibers (MERRF).

Leigh syndrome is a rare, inherited neurodegenerative condition. It usually becomes apparent in infancy, often after a viral infection, and symptoms usually progress rapidly. Early symptoms may include poor sucking ability, loss of head control and motor skills, loss of appetite, vomiting, and seizures. As the condition progresses, symptoms may include weakness and lack of muscle tone, spasticity, movement disorders, cerebellar ataxia, and peripheral neuropathy. Leigh syndrome can be due to mutations in either mitochondrial DNA or nuclear DNA. In one embodiment, an effective amount of an extended-release formulation of a compound of Formula I-Formula III or its pharmaceutically acceptable salt thereof, including levcromakalim, is administered for the treatment of Leigh syndrome.

Complex II deficiency, which can vary greatly from severe life-threatening symptoms in infancy to muscle disease beginning in adulthood, can be caused by mutations in the SDHA, SDHB, SDHD, or SDHAF1 genes. In one embodiment, an effective amount of an extended-release formulation of a compound of Formula I-Formula III or its pharmaceutically acceptable salt thereof, including levcromakalim, is administered for the treatment of Complex II deficiency. Complex III deficiency is a severe, multisystem disorder that includes features such as lactic acidosis, hypotonia, hypoglycemia, failure to thrive, encephalopathy, and delayed psychomotor development. Involvement of internal organs, including liver disease and renal tubulopathy, may also occur. It is generally caused by mutations in nuclear DNA in the BCSIL, UQCRB and UQCRQ genes and inherited in an autosomal recessive manner. However, it may also be caused by mutations in mitochondrial DNA in the MTCYB gene, which is passed down maternally or occurs sporadically and may result in a milder form of the condition. In one embodiment, an effective amount of an extended-release formulation of a compound of Formula I-Formula III or its pharmaceutically acceptable salt thereof, including levcromakalim, is administered for the treatment of Complex III deficiency.

Complex IV deficiency, also known as Cytochrome C oxidase deficiency (COX deficiency), is a condition that can affect several parts of the body including the skeletal muscles, heart, brain and liver. There are four types of COX deficiency differentiated by symptoms and age of onset: benign infantile mitochondrial type, French-Canadian type, infantile mitochondrial myopathy type, and Leigh syndrome. Complex IV deficiency is caused by mutations in any of at least 14 genes and the inheritance pattern depends on the gene involved. In one embodiment, an effective amount of an extended-release formulation of a compound of Formula I-Formula III or its pharmaceutically acceptable salt thereof, including levcromakalim, is administered for the treatment of Complex IV deficiency.

There are many other types of mitochondrial diseases. For example, dominant optic atrophy (DOA) is an inherited optic nerve disorder characterized by degeneration of the optic nerves that typically starts during the first decade of life. Affected people usually develop moderate visual loss and color vision defects. The severity varies and visual acuity can range from normal to legal blindness. Autosomal dominant optic atrophy plus syndrome (ADOA plus) is a rare syndrome that causes vision loss, hearing loss, and symptoms affecting the muscles. The syndrome is associated with optic atrophy. Other symptoms of ADOA plus include sensorineural hearing loss and symptoms affecting the muscles such as muscle pain and weakness. ADOA plus is caused by mutations in the OPA1 gene. Both DOA and ADOA are inherited in an autosomal dominant manner. In certain embodiments, an effective amount of an extended-release formulation of a compound of Formula I-Formula III or its pharmaceutically acceptable salt thereof, including levcromakalim, is administered for the treatment of dominant optic atrophy (DOA) or autosomal dominant optic atrophy plus syndrome (ADOA plus).

Alpers syndrome is a progressive neurologic disorder that begins during childhood and is complicated in many instances by serious liver disease. Symptoms include increased muscle tone with exaggerated reflexes (spasticity), seizures, and dementia. Most often Alpers syndrome is caused by mutations in the POLG gene. In one embodiment, an effective amount of an extended-release formulation of a compound of Formula I-Formula III or its pharmaceutically acceptable salt thereof, including levcromakalim, is administered for the treatment of Alpers syndrome.

Barth syndrome is a metabolic and neuromuscular disorder occurring almost exclusively in males that primarily affects the heart, immune system, muscles, and growth. It typically becomes apparent during infancy or early childhood. The main characteristics of the condition include abnormalities of heart and skeletal muscle (cardiomyopathy and skeletal myopathy); low levels of certain white blood cells called neutrophils that help to fight bacterial infections (neutropenia); and growth retardation that potential leads to short stature. Other signs and symptoms may include increased levels of certain organic acids in the urine and blood (such as 3-methylglutaconic acid) and increased thickness of the left ventricle of the heart due to endocardial fibroelastosis, which can cause potential heart failure. Barth syndrome is caused by mutations in the TAZ gene and is inherited in an X-linked recessive manner. In one embodiment, an effective amount of an extended-release formulation of a compound of Formula I-Formula III or its pharmaceutically acceptable salt thereof, including levcromakalim, is administered for the treatment of Barth syndrome.

Mitochondrial fatty acid β-oxidation disorders (FAODs) are a heterogeneous group of defects in fatty acid transport and mitochondrial β-oxidation. They are inherited as autosomal recessive disorders and have a wide range of clinical presentations. In one embodiment, an effective amount of an extended-release formulation of a compound of Formula I-Formula III or its pharmaceutically acceptable salt thereof, including levcromakalim, is administered for the treatment of a mitochondrial fatty acid β-oxidation disorders (FAOD). FAODs include CPT I deficiency, CACT deficiency, CPT II deficiency, LCAD deficiency, LCHAD deficiency, VLCAD deficiency, MCAD deficiency, SCHAD deficiency, and SCAD deficiency.

Primary carnitine deficiency is a genetic condition that prevents the body from using certain fats for energy, particularly during periods of fasting. The nature and severity of signs and symptoms may vary, but they most often appear during infancy or early childhood and can include severe brain dysfunction (encephalopathy), cardiomyopathy, confusion, vomiting, muscle weakness, and hypoglycemia. The condition is caused by mutations in the SLC22A5 gene and is inherited in an autosomal recessive manner. In one embodiment, an effective amount of an extended-release formulation of a compound of Formula I-Formula III or its pharmaceutically acceptable salt thereof, including levcromakalim, is administered for the treatment of primary carnitine deficiency.

Guanidinoacetate methyltransferase deficiency is an inherited disease that affects the brain and muscles. People with this disease may begin showing symptoms from early infancy to age three. Signs and symptoms can vary, but may include mild to severe intellectual disability, recurrent seizures, problems with speech, and involuntary movements. GAMT deficiency is caused by mutations in the GAMT gene. The disease is inherited in an autosomal recessive manner.

In one embodiment, an effective amount of an extended-release formulation of a compound of Formula I-Formula III or its pharmaceutically acceptable salt thereof, including levcromakalim, is administered for the treatment of guanidinoacetate methyltransferase deficiency.

Primary coenzyme Q10 deficiency involves a deficiency of coenzyme Q10 and can affect many parts of the body, especially the brain, muscles, and kidneys. The mildest cases of primary coenzyme Q10 deficiency can begin as late as a person's sixties and often cause cerebellar ataxia, which refers to problems with coordination and balance due to defects in the cerebellum. In one embodiment, an effective amount of an extended-release formulation of a compound of Formula I-Formula III or its pharmaceutically acceptable salt thereof, including levcromakalim, is administered for the treatment of primary coenzyme Q10 deficiency.

Chronic progressive external ophthalmoplegia (CPEO) is a condition characterized mainly by a loss of the muscle functions involved in eye and eyelid movement. Signs and symptoms tend to begin in early adulthood and most commonly include weakness or paralysis of the muscles that move the eye (ophthalmoplegia) and drooping of the eyelids (ptosis). Some affected individuals also have myopathy, which may be especially noticeable during exercise. CPEO can be caused by mutations in any of several genes, which may be located in mitochondrial DNA or nuclear DNA. CPEO can occur as part of other underlying conditions, such as ataxia neuropathy spectrum and Kearns-Sayre syndrome (KSS). KSS is a slowly progressive multi-system mitochondrial disease that often begins with ptosis. Other eye muscles eventually become involved, resulting in paralysis of eye movement. Degeneration of the retina usually causes difficulty seeing in dimly lit environments. In certain embodiments, an effective amount of an extended-release formulation of a compound of Formula I-Formula III or its pharmaceutically acceptable salt thereof, including levcromakalim, is administered for the treatment of chronic progressive external ophthalmoplegia or Kearns-Sayre syndrome.

Congenital lactic acidosis (CLA) is caused by mutations in mitochondrial DNA (mtDNA) that cause too much lactic acid to build up in the body, a condition called lactic acidosis. Severe cases of CLA manifest in the neonatal period, while milder cases caused by mtDNA mutations may not manifest until as late as early adulthood. Symptoms in the neonatal period include hypotonia, lethargy, vomiting, and tachypnea. As the disease progresses, it causes developmental delay, cognitive disabilities, abnormal development of the face and head, and organ failure. In one embodiment, an effective amount of an extended-release formulation of a compound of Formula I-Formula III or its pharmaceutically acceptable salt thereof, including levcromakalim, is administered for the treatment of congenital lactic acidosis (CLA).

Leukoencephalopathy with brain stem and spinal cord involvement and lactate elevation (LBSL) is a rare neurological disease characterized by slowly progressive cerebellar ataxia (lack of control of the movements) and spasticity with dorsal column dysfunction (decreased position and vibration sense) in most patients. The disease usually starts in childhood or adolescence, but in some cases not until adulthood. Symptoms may include difficulty speaking, epilepsy, learning problems, cognitive decline, and reduced consciousness, neurologic deterioration, and fever following minor head trauma. In one embodiment, an effective amount of an extended-release formulation of a compound of Formula I-Formula III or its pharmaceutically acceptable salt thereof, including levcromakalim, is administered for the treatment of leukoencephalopathy with brain stem and spinal cord involvement and lactate elevation (LBSL).

Leber hereditary optic neuropathy (LHON) is a condition characterized by vision loss. Some affected individuals develop features similar to multiple sclerosis. LHON is caused by mutations in the MT-ND1, MT-ND4, MT-ND4L, and MT-ND6 genes. In one embodiment, an effective amount of an extended-release formulation of a compound of Formula I-Formula III or its pharmaceutically acceptable salt thereof, including levcromakalim, is administered for the treatment of Leber hereditary optic neuropathy.

Glutaric acidemia type II (GA2) is a disorder that interferes with the body's ability to break down proteins and fats to produce energy. Most often, GA2 first appears in infancy or early childhood as a sudden episode of a metabolic crisis that can cause weakness, behavior changes (such as poor feeding and decreased activity) and vomiting. GA2 is inherited in an autosomal recessive manner and is caused by mutations in the ETFA, ETFB, or ETFDH genes. In one embodiment, an effective amount of an extended-release formulation of a compound of Formula I-Formula III or its pharmaceutically acceptable salt thereof, including levcromakalim, is administered for the treatment of Glutaric acidemia type II (GA2).

Mitochondrial enoyl CoA reductase protein associated neurodegeneration (MEPAN) is caused by 2 mutations in the gene MECR (which encodes the protein mitochondrial trans-2-enoyl-coenzyme A-reductase). Characteristics of MEPAN include optic atrophy and childhood-onset dystonia. In one embodiment, an effective amount of an extended-release formulation of a compound of Formula I-Formula III or its pharmaceutically acceptable salt thereof, including levcromakalim, is administered for the treatment of mitochondrial enoyl CoA reductase protein associated neurodegeneration (MEPAN).

Mitochondrial DNA (mtDNA) depletion syndrome (MDS) is a clinically heterogeneous group of mitochondrial disorders characterized by a reduction of the mtDNA copy number in affected tissues without mutations or rearrangements in the mtDNA. MDS is phenotypically heterogeneous and can affect a specific organ or a combination of organs, with the main presentations described being either hepatocerebral (i.e., hepatic dysfunction, psychomotor delay), myopathic (i.e., hypotonia, muscle weakness, bulbar weakness), encephalomyopathic (i.e., hypotonia, muscle weakness, psychomotor delay) or neurogastrointestinal (i.e., gastrointestinal dysmotility, peripheral neuropathy). There are generally four classes of MDDS: 1) a form that primarily affects muscle associated with mutations in the TK2 gene; 2) a form that primarily affects the brain and muscle associated with mutations in the genes SUCL42, SUCLG1, or RRM2B; 3) a form that primarily affects the brain and the liver associated with mutations in DGUOK, MPV17, POLG, or TWNK (also called PEO1); and, 4) a form that primarily affects the brain and the gastrointestinal tract associated with mutations in ECGF1 (also called TYMP). In one embodiment, an effective amount of an extended-release formulation of a compound of Formula I-Formula III or its pharmaceutically acceptable salt thereof, including levcromakalim, is administered for the treatment of a mitochondrial DNA (mtDNA) depletion syndrome (MDS).

Mitochondrial neurogastrointestinal encephalopathy (MNGIE) disease is a condition that affects several parts of the body, particularly the digestive system and nervous system. The major features of MNGIE disease can appear at any point from infancy to adulthood, but signs and symptoms most often begin by age twenty. MNGIE disease is also characterized by abnormalities of the nervous system, although these tend to be milder than the gastrointestinal problems.

Affected individuals experience tingling, numbness, and weakness in their limbs (peripheral neuropathy), particularly in the hands and feet. Additional neurological signs and symptoms can include droopy eyelids (ptosis), weakness of the muscles that control eye movement (ophthalmoplegia), and hearing loss. Leukoencephalopathy, which is the deterioration of a type of brain tissue known as white matter, is a hallmark of MNGIE disease. In one embodiment, an effective amount of an extended-release formulation of a compound of Formula I-Formula III or its pharmaceutically acceptable salt thereof, including levcromakalim, is administered for the treatment of mitochondrial neurogastrointestinal encephalopathy (MNGIE).

Neuropathy ataxia retinitis pigmentosa (NARP) syndrome is characterized by a variety of signs and symptoms that mainly affect the nervous system. Beginning in childhood or early adulthood, most people with NARP experience numbness, tingling, or pain in the arms and legs (sensory neuropathy), muscle weakness, and problems with balance and coordination (ataxia).

Affected individuals may also have vision loss caused by a condition called retinitis pigmentosa.

Mutations in the MT-ATP6 gene cause NARP syndrome. In one embodiment, an effective amount of an extended-release formulation of a compound of Formula I-Formula III or its pharmaceutically acceptable salt thereof, including levcromakalim, is administered for the treatment of neuropathy ataxia retinitis pigmentosa (NARP) syndrome.

Pearson syndrome affects many parts of the body, but especially the bone marrow and the pancreas. Pearson syndrome affects the cells in the bone marrow (hematopoietic stem cells) that produce red blood cells, white blood cells, and platelets. Pearson syndrome also affects the pancreas, which can cause frequent diarrhea and stomach pain, trouble gaining weight, and diabetes. Some children with Person syndrome may also have problems with their liver, kidneys, heart, eyes, ears, and/or brain. Pearson syndrome is caused by a mutation in the mitochondrial DNA. In one embodiment, an effective amount of an extended-release formulation of a compound of Formula I-Formula III or its pharmaceutically acceptable salt thereof, including levcromakalim, is administered for the treatment of Pearson syndrome.

POLG-related disorders comprise a continuum of overlapping phenotypes with onset from infancy to late adulthood. Mutations in POLG can cause early childhood mitochondrial DNA (mtDNA) depletion syndromes or later-onset syndromes arising from mtDNA deletions. POLG mutations are the most common cause of inherited mitochondrial disorders, with as many as 2% of the population carrying these mutations. The six leading disorders caused by POLG mutations are Alpers-Huttenlocher syndrome, which is one of the most severe phenotypes; childhood myocerebrohepatopathy spectrum, which presents within the first three years of life; myoclonic epilepsy myopathy sensory ataxia; ataxia neuropathy spectrum (which includes the phenotypes previously referred to as mitochondrial recessive ataxia syndrome (MIRAS) and sensory ataxia neuropathy dysarthria and ophthalmoplegia (SANDO)); autosomal recessive progressive external ophthalmoplegia; and, autosomal dominant progressive external ophthalmoplegia. In one embodiment, an effective amount of an extended-release formulation of a compound of Formula I-Formula III or its pharmaceutically acceptable salt thereof, including levcromakalim, is administered for the treatment of a POLG-related disorder.

Pyruvate carboxylase deficiency is an inherited disorder that causes lactic acid and other potentially toxic compounds to accumulate in the blood. High levels of these substances can damage the body's organs and tissues, particularly in the nervous system. There are at least three types of pyruvate carboxylase deficiency, types A, B, and C, which are distinguished by the severity of their signs and symptoms. This condition is caused by mutations in the PC gene and inherited in an autosomal recessive pattern. In one embodiment, an effective amount of an extended-release formulation of a compound of Formula I-Formula III or its pharmaceutically acceptable salt thereof, including levcromakalim, is administered for the treatment of pyruvate carboxylase deficiency.

Pyruvate dehydrogenase complex (PDC) deficiency is a type of metabolic disease where the body is not able to efficiently break down nutrients in food to be used for energy. Symptoms of PDC deficiency include signs of metabolic dysfunction such as extreme tiredness (lethargy), poor feeding, and rapid breathing (tachypnea). Other symptoms may include signs of neurological dysfunction such as developmental delay, periods of uncontrolled movements (ataxia), low muscle tone (hypotonia), abnormal eye movements, and seizures. Symptoms usually begin in infancy, but signs can first appear at birth or later in childhood. The most common form of PDC deficiency is caused by genetic (mutations or pathogenic variants in the PDHA1 gene. In one embodiment, an effective amount of an extended-release formulation of a compound of Formula I-Formula III or its pharmaceutically acceptable salt thereof, including levcromakalim, is administered for the treatment of pyruvate carboxylase deficiency.

TK2-Related mitochondrial DNA depletion syndrome, myopathic form (TK2-MDS) is an inherited condition that causes progressive myopathy. The signs and symptoms of TK2-MDS typically begin in early childhood. Development is usually normal early in life, but as muscle weakness progresses, people with TK2-MDS lose motor skills such as standing, walking, eating, and talking. Some affected individuals have increasing weakness in the muscles that control eye movement, leading to droopy eyelids (progressive external ophthalmoplegia). In one embodiment, an effective amount of an extended-release formulation of a compound of Formula I-Formula III or its pharmaceutically acceptable salt thereof, including levcromakalim, is administered for the treatment of TK2-related mitochondrial DNA depletion syndrome, myopathic form (TK2-MDS).

### Selected Ocular Disorders

In additional aspects of the invention, an extended-release formulation of a compound or a pharmaceutically acceptable salt of Formula I-III, including levcromakalim, is used for the treatment of a selected ocular disorder, as described below.

Graves' ophthalmopathy or Graves' orbitopathy (or thyroid eye disease or thyroid-associated orbitopathy) are autoimmune inflammatory disorders of the orbit and periorbital tissues and typical signs of the diseases include upper eyelid retraction, lid lag, swelling, and bulging eyes.

These disorders are orbital autoimmune disorders caused by an overactive thyroid. An effective amount of an extended-release formulation of a compound of Formula I-III can be administered for the treatment of Graves' ophthalmopathy, Graves' orbitopathy, or thyroid-associated orbitopathy. The compound can be administered in any manner that achieves the desired effect, including as a topical drop taken as needed to reduce swelling and redness. In one embodiment, an extended-release formulation of a compound of Formula I-III is taken in combination with a corticosteroid drug or an immune suppression medication (rituximab or mycophenolate).

Orbital tumors are benign or malignant space-occupying lesions of the orbit, often leading to dystopia of the eyeball, motility disturbances, diplopia, visual field defects, and sometimes a complete loss of vision. Often orbital tumors are removed via surgery and therefore a medication would be an advantageous therapeutic option. In one embodiment, an effective amount of an extended-release formulation of a compound or a pharmaceutically acceptable salt of Formula I-Formula III, including levcromakalim, is administered for the treatment or reduction of orbital tumors. In one embodiment, the compound is administered topically one time, two times, three times, or more a day. In one embodiment, the compound is administered prior to or after surgery for the removal or reduction of orbital tumors.

Cavernous sinus thrombosis is the formation of a blood clot within the cavernous sinus, a cavity at the base of the brain which drains deoxygenated blood from the brain back to the heart.

This is a rare disorder and can be of two types: septic cavernous thrombosis and aseptic cavernous thrombosis. The cause is often secondary to an infection in the nose, sinuses, ears, or teeth. A common disorder secondary to cavernous sinus pathology is superior ophthalmic vein thrombosis, an uncommon orbital pathology that can present with sudden onset proptosis, conjunctival injection, and visual disturbance.

In one embodiment, an effective amount of an extended-release formulation of a compound or a pharmaceutically acceptable salt of Formula I-III, including levcromakalim, is administered for the treatment of cavernous sinus thrombosis or superior ophthalmic vein thrombosis. In one embodiment, an effective amount is administered in combination or alternation with an antibiotic, heparin, or a steroid. In one aspect, the compound is administered orally and is given at least once, twice, three, or more times a day as needed.

Episcleral/orbital vein vasculitis is inflammation of the blood vessel wall. The clinical features of the eye vasculitis can vary from conjunctivitis, episcleritis, scleritis, peripheral ulcerative keratitis, proptosis, retinal vasculitis, orbititis to uveitis, depending on the site and distribution of the vessels involved. In one embodiment, an effective amount of an extended-release formulation of a compound or a pharmaceutically acceptable salt of Formula I-III, including levcromakalim, is administered for the treatment of episcleral/orbital vein vasculitis. In one embodiment, the prodrug is administered as a topical drop.

Carotid-cavernous sinus fistula is an abnormal connection between an artery in the neck and the network of veins at the back of the eye. A fistula can raise the pressure in your cavernous sinuses, which may compress the cranial nerves located around the cavernous sinuses. This compression may damage the nerve function, which is to control your eye movements. Carotid-cavernous sinus fistula can be direct or indirect. Direct carotid-cavernous sinus fistulas are often caused by accidents or injuries that tear the carotid artery wall, while indirect carotid-cavernous sinus fistulas often arise without warning and are associated with high blood pressure, hardened arteries, pregnancy, and connective tissue disorders. In one embodiment, an effective amount of an extended-release formulation of a compound or a pharmaceutically acceptable salt of Formula I-III, including levcromakalim, is administered for the treatment of carotid-cavernous sinus fistula. In one embodiment, the extended-release formulation is administered as an oral dosage form.

Dural cavernous sinus shunts are vascular communications in which blood flows through small meningeal branches of the carotid arteries to enter the venous circulation near the cavernous sinus. Often this disorder is congenital, and the onset of clinical abnormalities may be associated with the occurrence of intracranial venous thrombosis. In one embodiment, an effective amount of an extended-release formulation of a compound or a pharmaceutically acceptable salt of Formula I-III, including levcromakalim, is administered for the treatment of dural cavernous sinus shunts.

In one embodiment, the prodrug is administered as an oral dosage form.

Orbital varices are a vascular hamartoma typified by a plexus of low pressure, low flow, thin walled and distensible vessels that intermingle with the normal orbital vessels. Most patients will experience positional proptosis with a head-down position, and intermittent proptosis that is exacerbated by coughing, straining, the Valsalva maneuver, or compression of the jugular veins.

In one embodiment, an extended-release formulation of a compound or a pharmaceutically acceptable salt of Formula I-III, including levcromakalim, is administered for the treatment of orbital varices. In one embodiment, the prodrug is administered as an oral dosage form.

Sturge-Weber Syndrome is a condition that affects the development of certain blood vessels, causing abnormalities in the brain, skin, and eyes from birth. Sturge-Weber Syndrome has three major features: a red or pink birthmark called a port-wine birthmark, a brain abnormality called a leptomeningeal angioma, and increased IOP in the eye (glaucoma). In individuals with Sturge-Weber Syndrome, glaucoma typically develops either in infancy or early adulthood and can cause vision impairment. In some affected infants, the pressure can become so great that the eyeballs appear enlarged and bulging (buphthalmos). Individuals with Sturge-Weber Syndrome can have tangles of abnormal blood vessels (hemangiomas) in various parts of the eye. When these abnormal blood vessels develop into a network of blood vessels at the back of the eye (choroid), it is called a diffuse choroidal hemangioma and occurs in about one-third of individuals with Sturge-Weber Syndrome. A diffuse choroidal hemangioma can cause vision loss. When present, the eye abnormalities typically occur on the same side of the head as the port-wine birthmark.

In one embodiment, an effective amount of an extended-release formulation of a compound or a pharmaceutically acceptable salt of Formula I-III, including levcromakalim, is administered for the treatment of Sturge-Weber Syndrome. In one embodiment, an effective amount of an extended-release formulation of a compound or a pharmaceutically acceptable salt of Formula I-III, including levcromakalim, is administered for the treatment of Sturge-Weber Syndrome-induced glaucoma. In one embodiment, the compound is administered as an oral formulation once, twice, three, or more times a day. In one embodiment, the extended-release formulation is administered as a topical ocular formulation and is administered once a day for long term therapy, as defined herein.

Central retinal vein occlusion, also known as CRVO, is a condition in which the main vein that drains blood from the retina becomes blocked partially or completely. This can cause blurred vision and other problems with the eye. Risk factors for CRVO include diabetes, elevated IOP, and high blood pressure. The macula can swell from this fluid, affecting central vision. Eventually, without blood circulation, nerve cells in the eye can die and vision loss can occur. In one embodiment, an effective amount of an extended-release formulation of a compound or a pharmaceutically acceptable salt of Formula I-III, including levcromakalim, is administered for the treatment of central retinal vein occlusion. In one embodiment, the compound is administered as a topical drop that is given once, twice, or three times a day. In one embodiment, the prodrug is given in combination with an anti-VEGF inhibitor such as bevacizumab (Avastin®), ranibizumab (Lucentis®), aflibercept (Eylea®), and brolucizumab (Beovu®).

Branch retinal vein occlusion (BRVO) is the blockage of branches of the retinal vein causing blood and fluid to spill into the retina. Risk factors for BRVO include diabetes, elevated IOP, and high blood pressure. The macula can swell from this fluid, affecting central vision.

Eventually, without blood circulation, nerve cells in the eye can die and vision loss can occur. In one embodiment, an effective amount of an extended-release formulation of a compound or a pharmaceutically acceptable salt of Formula I-III, including levcromakalim, is administered for the treatment of branch retinal vein occlusion (BRVO). In one embodiment, the prodrug is administered as a topical drop that is given once, twice, three, or more times a day.

Non-arteritic anterior ischemic optic neuropathy (NAION) refers to loss of blood flow to the optic nerve and is due to impaired circulation of blood at the optic nerve head. Non-arteritic anterior ischemic optic neuropathy is associated with diabetes, high blood pressure, atherosclerosis, a small optic nerve, elevated IOP, and sleep apnea. In one embodiment, an effective amount of an extended-release formulation of a compound or a pharmaceutically acceptable salt of Formula I-III, including levcromakalim, is administered for the treatment of non-arteritic anterior ischemic optic neuropathy. In one embodiment, the prodrug is administered as a topical drop that is given once, twice, three, or more times a day.

In some embodiments, an effective amount of an extended-release formulation of a compound or a pharmaceutically acceptable salt of Formula I-III, including levcromakalim, is used as a secondary treatment to latanoprost for the treatment of an ocular disorder as described herein.

In some embodiments, in may be useful to administer an extended-release formulation of a compound or a pharmaceutically acceptable salt of Formula I-III, including levcromakalim, to a host in need thereof in combination with, for example,
(1) a prostaglandin analog, such as latanoprost (Xalatan), bimatoprost (Lumigan), travoprost (Travatan or Travatan Z), or Tafluprost (Zioptan);
(2) an α-2 adrenergic agonist, such as brimonidine (Alphagan®), epinephrine, dipivefrin (Propine®) or apraclonidine (Lopidine®));
(3) a beta-blocker, such as timolol, levobunolol, metipranolol, or carteolol;
(4) a ROCK inhibitor, such as ripasudil, netarsudil (Rhopressa), fasudil, RKI-1447, GSK429286A, or Y-30141;
(5) a second potassium channel opener, such as minoxidil, diazoxide, nicorandil, or pinacidil;
(6) a carbonic anhydrase inhibitor, such as dorzolamide (Trusopt®), brinzolamide (Azopt®), acetazolamide (Diamox®) or methazolamide (Neptazane®);
(7) a PI3K inhibitor, such as Wortmannin, demethoxyviridin, perifosine, idelalisib, Pictilisib, Palomid 529, ZSTK474, PWT33597, CUDC-907, and AEZS-136, duvelisib, GS-9820, BKM120, GDC-0032 (Taselisib) (2-[4-[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[l-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N-[4-(4,6-di-4-morpholinyl-1,3,5-triazin-2-yl)phenyl]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl] acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422);
(8) a BTK inhibitor, such as: ibrutinib (also known as PCI-32765)(Imbruvica™)(1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (US Patent publication No 2011/0117073, incorporated herein in its entirety), Dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-ylamino]-6-oxo-1,6-dihydro-pyridin-3-yl}-phenyl)-2H-isoquinolin-1-one); or a
(9) a Syk inhibitor, such as Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306, 7-azaindole, piceatannol, ER-27319, PRT060318, luteolin, apigenin, quercetin, fisetin, myricetin, morin.

In alternative embodiments, an extended-release formulation of a compound or a pharmaceutically acceptable salt of Formula I-III, including levcromakalim, is administered to a host in need thereof in combination with a nitric oxide donor, including, but not limited to, NCX-470, NCX-1728, NCX-4251, NCX-4016, NCX-434, NCX-667, Vyzulta (latanoprostene bunod ophthalmic solution), or sodium nitroprusside (SNP).

### Ophthalmic Neuroprotection

Neuroprotection is a therapeutic strategy with the goal of maximizing the recovery of neural cells and minimizing neuronal cell death due to injury. The injury can be mechanical, ischemic, degenerative, or radiation. Many neurodegenerative disorders are associated with aging, which can be detrimental for the elderly population. For example, glaucoma is often characterized by the loss of retinal ganglion cells and is a major cause of vision loss and blindness in the elderly.

In one embodiment, an extended-release formulation of a compound or a pharmaceutically acceptable salt of Formula I-III, including levcromakalim, is administered to a host in need thereof for the treatment of an ocular-related neurodegenerative disorder. An ocular-related neurodegenerative disorder is any disorder that is associated with the dysfunction or degeneration of neurons or cells, including neural cells, such as retinal ganglion cells.

In one embodiment of the present invention, an extended-release formulation of a compound or a pharmaceutically acceptable salt of Formula I-III, including levcromakalim, is administered as a method for reducing neuronal or cellular damage in the eye of host in need thereof. In one embodiment, an extended-release formulation of a compound or a pharmaceutically acceptable salt of Formula I-III, including levcromakalim, is administered as a method for reducing neuronal or cellular damage in the eye of host in need thereof wherein the eye is glaucomatous.

In another embodiment, an extended-release formulation of a compound or a pharmaceutically acceptable salt of Formula I-III, including levcromakalim, promotes the survival, growth, regeneration, and/or neurite outgrowth of retinal ganglion cells. In another embodiment, an extended-release formulation of a compound or a pharmaceutically acceptable salt of Formula I-III, including levcromakalim, prevents the death of damaged neuronal cells.

Neuronal cell death can also be a result of retinal ischemia, and therefore in one embodiment, an extended-release formulation of a compound or a pharmaceutically acceptable salt of Formula I-III, including levcromakalim, is administered as a method of reducing neuronal or cellular damage in the eye following retinal ischemia in a host in need thereof.

Optic neuropathy, which is damage to the optic nerve often characterized by visual loss, results in the loss of retinal ganglion cells. There are many types of optic neuropathies, including ischemic optic neuropathy, optic neuritis, compressive optic neuropathy, infiltrative optic neuropathy, and traumatic optic neuropathy. Nutritional optic neuropathy can also result from under nutrition and/or a vitamin B12 deficiency. Toxic optic neuropathy can result from exposure to ethylene glycol, methanol, ethambutol, amiodarone, tobacco, or certain drugs, such as chloramphenicol or digitalis. Certain forms of optic neuropathy can be inherited, including Leber's hereditary optic neuropathy (LHON), dominant optic atrophy, Behr's syndrome, and Berk-Tabatznik syndrome. In one embodiment, an effective amount of an extended-release formulation of a compound or a pharmaceutically acceptable salt of Formula I-III, including levcromakalim, is administered as a method for reducing as a method of reducing neuronal or cellular damage in the eye of a host in need thereof with optic neuropathy.

Additional non-limiting examples of ocular-related neurodegenerative diseases include lattice dystrophy, retinitis pigmentosa, age-related macular degeneration (wet or dry), photoreceptor degeneration associated with wet- or dry-age related macular degeneration, and optic nerve drusen.

Integrated or Adjunctive Therapy with Microinvasive Glaucoma Surgery (MIGS)

Minimally (or Micro) Invasive Glaucoma Surgery (MIGS) has become an innovative procedure in the evolution of glaucoma surgery. Since glaucoma is a disease in which the optic nerve gets damaged primarily due to elevated IOP, the goal of glaucoma surgery is to lower IOP to prevent or reduce damage to the optic nerve.

Standard glaucoma surgeries are still considered a major surgery and involve trabeculectomy, ExPRESS shunts, or external tube-shunts such as the Ahmed, Molteno, and Baerveldt style valve implants. While such procedures have often been effective at lowering eye pressure and preventing progression of glaucoma, they have numerous potential complications such as double vision, devastating eye infections, exposure of a drainage implant, swelling of the cornea, and excessively low IOP.

According to Saheb and Ahmed, minimally (or micro) invasive glaucoma surgery refers to a group of procedures which share five preferable qualities:
1. an ab interno and/or ab externo approach through a clear corneal incision which may spare the conjunctiva of incision;
2. a minimally traumatic procedure to the target tissue;
3. an IOP lowering efficacy that justifies the approach;
4. a high safety profile avoiding serious complications compared to other glaucoma surgeries, and given lower likelihood of hypotony; and
5. a rapid recovery with minimal impact on the patient's quality of life.

The MIGS group of operations have been developed in recent years to reduce some of the complications of most standard glaucoma surgeries and therefore, in one embodiment, an extended-release formulation of a compound of Formula I-Formula III is used as an additive in combination with a microinvasive glaucoma surgery (MIGS).

MIGS is intended to achieve lower IOP in patients with glaucoma with a less invasive surgical procedure, and ideally to achieve a medication sparing effect. MIGS procedures work by using microscopic-sized equipment and tiny incisions, enable controlled outflow and are often conducted at the time of cataract surgery. While they reduce the incidence of complications, some degree of effectiveness is traded for the increased safety. (Pillunat, L. E., et al., Clin Ophthalmol. 2017; 11: 1583-1600)

The MIGS group of operations are divided into several categories:
1. Trabecular bypass operations (i.e., angle-based devices and or subconjunctival shunting devices);
2. Microtrabeculectomies (miniaturized versions of trabeculectomy);
3. Totally internal or suprachoroidal shunts; and,
4. Milder, gentler versions of laser photocoagulation.

Trabecular Surgery (Trabeculotomy) involves the use of a special contact lens on the eye and cutting through the trabecular meshwork with a tiny device under high power microscopic control. This is done without damaging any other tissues in the ocular drainage pathway. The trabecular meshwork can either be destroyed (Trabectome or Trab360) or bypassed using a tiny snorkel-like device (the iStent) or using a plug-shaped stent device (iStent Inject). Both procedures are FDA-approved but generally do not reduce eye pressure low enough and are thus useful in early to moderate stages of glaucoma. With these devices, the resistance of the trabecular meshwork is obviated, thus primarily leaving distal outflow facility and episcleral venous pressure as limits to further aqueous humor drainage. In certain embodiments, an extended-release formulation of a compound or a pharmaceutically acceptable salt of Formula I-III, including levcromakalim, is used as an additive in combination with Trabectome or Trab360 and/or the iStent/iStent Inject for the treatment of glaucoma by additively lowering IOP via increased distal outflow or reduced episcleral venous pressure prior to or after the procedure in an acute or chronic use setting.

Microtrabeculectomies work by inserting tiny, microscopic-sized tubes into the eye and draining the fluid from inside the eye to underneath the outer membrane of the eye (conjunctiva). The Xen Gel Stent and PRESERFLO are two new devices that can make the trabeculectomy operation safer. Results have shown excellent pressure lowering with improved safety over trabeculectomy in studies done outside the United States. In certain embodiments, the compounds of the present invention are used as part of the protocols with Xen Gel Stent and/or Preserflo for the treatment of glaucoma by additively lowering IOP via increased distal outflow or reduced episcleral venous pressure prior to or after the procedure in an acute or chronic use setting.

Suprachoroidal Shunts, including the Gold Micro-shunt, iStent Supra, Aquashunt, and STARflo, work by using tiny tubes with very small internal openings, the front of the eye is connected to the suprachoroidal space between the retina and the wall of the eye to augment the drainage of fluid from the eye. This operation has relatively few serious complications and lowers pressures enough to be useful even in moderately severe glaucoma. In certain embodiments, an extended-release formulation of a compound or a pharmaceutically acceptable salt of Formula I-III, including levcromakalim, is used in combination with Suprachoroidal Shunts procedure for the treatment of glaucoma by additively lowering IOP via increased distal outflow or reduced episcleral venous pressure prior to or after the procedure in an acute or chronic use setting.

Trabecular bypass stents and shunts are investigational devices that work to dilate Schlemm's canal. These procedures facilitate the flow of aqueous into Schlemm's canal by shunting (Eyepass Glaucoma Implant; GMP Companies, Inc., Fort Lauderdale, FL) or by stenting the canal itself (iStent; Glaukos Corp., Laguna Hills, CA). Other devices such as the Solx Gold Micro-Shunt (OccuLogix, Inc., Mississauga, Ontario, Canada) divert aqueous into the suprachoroidal space. In certain embodiments, an extended-release formulation of a compound or a pharmaceutically acceptable salt of Formula I-III, including levcromakalim, is used in combination with trabecular bypass stents or shunts procedure for the treatment of glaucoma by additively lowering IOP via increased distal outflow or reduced episcleral venous pressure prior to or after the procedure in an acute or chronic use setting.

Selective laser trabeculoplasty (SLT) is used any during the management to help lower IOP. Since the conduct of the LiGHT study, it has now been used more often as first line-treatment to help lower IOP, effectively working at the level of the trabecular meshwork to improve outflow.

In certain embodiments, an extended-release formulation of a compound or a pharmaceutically acceptable salt of Formula I-III, including levcromakalim, is used alongside and/or in addition to SLT for the treatment of glaucoma by additively lowering IOP via increased distal outflow and/or reduced episcleral venous pressure prior to or after the procedure in an acute or chronic use setting.

Laser photocoagulation was previously reserved for advanced glaucoma that could not be controlled despite trabeculectomy or tube shunts. Endocyclophotocoagulation and micropulse Diode cyclophotocoagulation are two recent advances to the use of laser photocoagulation and have proven useful in cases where glaucoma has yet to become advanced. In certain embodiments, an extended-release formulation of a compound or a pharmaceutically acceptable salt of Formula I-II, including levcromakalim, is used in the endocyclophotocoagulation and micropulse cyclophotocoagulation protocol for the treatment of glaucoma by additively lowering IOP via increased distal outflow and/or reduced episcleral venous pressure prior to or after the procedure in an acute or chronic use setting.

Endocyclophotocoagulation in recent years has become a widely accepted and popular treatment of refractory glaucoma, pediatric glaucoma, and as an adjunct to cataract surgery in both medically controlled and uncontrolled glaucoma in conjunction with phacoemulsification with intraocular lens placement. Endocyclophotocoagulation is performed following lens removal and intraocular lens implantation by inserting an endolaser unit through the cataract incision, across the anterior segment, and into the posterior chamber on the nasal side of the eye. Laser energy is applied to the ciliary processes to destroy ciliary epithelial cells that produce aqueous humor. In certain embodiments, an extended-release formulation of a compound or a pharmaceutically acceptable salt of Formula I-III, including levcromakalim, is used in the endocyclophotocoagulation protocol for the treatment of glaucoma by additively lowering IOP via increased distal outflow and/or reduced episcleral venous pressure prior to or after the procedure in an acute or chronic use setting.

Micropulse cyclophotocoagulation delivers the laser in short bursts to allow the surgeon to target specific areas of the ciliary body while giving the tissue time to cool down between bursts, minimizing damage. MicroPulse P3 probe and the new Cyclo G6 glaucoma laser system (Iridex) have both been used successfully in retinal diseases, showing excellent safety and efficacy rates.

In certain embodiments, an extended-release formulation of a compound or a pharmaceutically acceptable salt of Formula I-III, including levcromakalim, is used in the Micropulse cyclophotocoagulation surgical protocol for the treatment of glaucoma by additively lowering IOP via increased distal outflow and/or reduced episcleral venous pressure prior to or after the procedure in an acute or chronic use setting.

Other devices include Gonioscopy-assisted transluminal trabeculotomy (GATT), Kahook Dual Blade, Ab interno canaloplasty and Hydrus Microstent, iStent Supra, Xen Glaucoma Treatment System and InnFocus MicroShunt. In certain embodiments, an extended-release formulation of a compound or a pharmaceutically acceptable salt of Formula I-III, including levcromakalim, is used in the surgical protocol of these devices for the treatment of glaucoma as described above.

Laser Trabeculoplasty, including Selective Laser Trabeculoplasty (SLT), Argon Laser Trabeculoplasty (ALT), Excimer Laser Trabeculostomy and Micropulse Laser Trabeculoplasty (MLT) are surgical laser procedures that help to reduce resistance at the trabecular meshwork by ablating cells of the trabecular meshwork and improving outflow in a manner similar to other forms of trabeculoplasty and certain MIGS devices. In certain embodiments, Excimer Laser Trabeculostomy used as an additive in combination with Laser Trabeculoplasty for the treatment of glaucoma by additively lowering IOP via increased distal outflow or reduced episcleral venous pressure prior to or after the procedure in an acute or chronic use setting.

In one embodiment, an extended-release formulation of a compound or a pharmaceutically acceptable salt of Formula I-Formula III, including levcromakalim, is used as a secondary therapy to a prostaglandin analog, such as latanoprost (Xalatan), bimatoprost (Lumigan), travoprost (Travatan or Travatan Z), latanoprostene bunod (Vyzulta), or Tafluprost (Zioptan) and as an additive to a minimally (or micro) invasive glaucoma surgery (MIGS) as described herein. In a further embodiment, the MIGS is a trabeculotomy. In a further embodiment, the MIGS is a microtrabeculectomy. In a further embodiment, the MIGS is a suprachoroidal shunt. In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty.

In one embodiment, an extended-release formulation of a compound or a pharmaceutically acceptable salt of Formula I-Formula III, including levcromakalim, is used as a secondary therapy to latanoprost (Xalatan) and as an additive to a minimally (or micro) invasive glaucoma surgery as described herein. In a further embodiment, the MIGS is a trabeculotomy. In a further embodiment, the MIGS is a microtrabeculectomy. In a further embodiment, the MIGS is a suprachoroidal shunt. In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty.

In one embodiment, an extended-release formulation of a compound or a pharmaceutically acceptable salt of Formula I-Formula III, including levcromakalim, is used as a secondary therapy to an α-2 adrenergic agonist, such as brimonidine (Alphagan®), epinephrine, dipivefrin (Propine®) or apraclonidine (Lopidine®) and as an additive to a minimally (or micro) invasive glaucoma surgery (MIGS) as described herein. In a further embodiment, the MIGS is a trabeculotomy. In a further embodiment, the MIGS is a microtrabeculectomy. In a further embodiment, the MIGS is a suprachoroidal shunt. In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty.

In one embodiment, an extended-release formulation of a compound or a pharmaceutically acceptable salt of Formula I-Formula III, including levcromakalim, is used as a secondary therapy to a beta-blocker, such as timolol, levobunolol, metipranolol, or carteolol and as an additive to a minimally (or micro) invasive glaucoma surgery (MIGS) as described herein. In a further embodiment, the MIGS is a trabeculotomy. In a further embodiment, the MIGS is a microtrabeculectomy. In a further embodiment, the MIGS is a suprachoroidal shunt. In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty. In a further embodiment, the MIGS is a trabeculotomy. In a further embodiment, the MIGS is a microtrabeculectomy. In a further embodiment, the MIGS is a suprachoroidal shunt. In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty.

In one embodiment, an extended-release formulation of a compound or a pharmaceutically acceptable salt of Formula I-Formula III, including levcromakalim is used as a secondary therapy to a ROCK inhibitor, such as ripasudil, netarsudil (Rhopressa), fasudil, RKI-1447, GSK429286A, or Y-30141 and as an additive to a minimally (or micro) invasive glaucoma surgery (MIGS) as described herein. In a further embodiment, the MIGS is a trabeculotomy. In a further embodiment, the MIGS is a microtrabeculectomy. In a further embodiment, the MIGS is a suprachoroidal shunt.

In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty.

In one embodiment, an extended-release formulation of a compound or a pharmaceutically acceptable salt of Formula I-Formula III, including levcromakalim, is used as a secondary therapy to a second potassium channel opener, such as minoxidil, diazoxide, nicorandil, or pinacidil and as an additive to a minimally (or micro) invasive glaucoma surgery (MIGS) as described herein.

In a further embodiment, the MIGS is a trabeculotomy. In a further embodiment, the MIGS is a microtrabeculectomy. In a further embodiment, the MIGS is a suprachoroidal shunt. In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty.

In one embodiment, an extended-release formulation of a compound or a pharmaceutically acceptable salt of Formula I-Formula III, including levcromakalim is used as a secondary therapy to a carbonic anhydrase inhibitor, such as dorzolamide (Trusopt®), brinzolamide (Azopt®), acetazolamide (Diamox®) or methazolamide (Neptazane®) and as an additive to a minimally (or micro) invasive glaucoma surgery (MIGS) as described herein. In a further embodiment, the MIGS is a trabeculotomy. In a further embodiment, the MIGS is a microtrabeculectomy. In a further embodiment, the MIGS is a suprachoroidal shunt. In a further embodiment, the MIGS is a trabecular bypass stent or shunt. In a further embodiment, the MIGS is a selective laser trabeculoplasty (SLT). In a further embodiment, the MIGS is a laser photocoagulation. In a further embodiment, the MIGS is endocyclophotocoagulation. In a further embodiment, the MIGS is laser trabeculoplasty.

### III. Pharmaceutical Compositions and Dosage Forms

### Extended-Release Formulations

In some embodiments, extended release formulations of compounds of Formulas I, II or III, for example levcromakalim, or a pharmaceutically acceptable salt thereof of the present invention described herein, for example a microparticle or a nanoparticle, may include a biodegradable polymer for controlled delivery of the selected compound, including, but not limited to a pluronic polymer, a polyester (e.g., polylactic acid, poly(lactic-co-glycolic acid), polycaprolactone, polyvalerolactone, poly(1,3-dioxan-2-one)); polyanhydride (e.g., poly(sebacic anhydride)); polyether (e.g., polyethylene glycol); polyurethane; polymethacrylate; polyacrylate; and polycyanoacrylate. In some embodiments, the selected polymer may be modified, such as end-capped, with polyethylene glycol (PEG), with a carbohydrate, and/or with acyclic polyacetals derived from polysaccharides. See, e.g., Papisov, 2001, ACS Symposium Series, 786:301, incorporated by reference herein.

Techniques for the manufacture of microparticles and nanoparticles are well known. They include, but are not limited to, solvent evaporation, solvent removal, spray drying, phase inversion, coacervation, and low temperature casting. Suitable methods of particle formulation are briefly described below. Pharmaceutically acceptable excipients, including pH modifying agents, disintegrants, preservatives, and antioxidants, can optionally be incorporated into the particles during particle formation.

In some embodiments, the particles are derived through a solvent evaporation method. In this method, a compound described herein (or polymer matrix and one or more compounds described herein) is dissolved in a volatile organic solvent, such as methylene chloride. The organic solution containing a compound described herein is then suspended in an aqueous solution that contains a surface active agent such as poly(vinyl alcohol). The resulting emulsion is stirred until most of the organic solvent is evaporated, leaving solid nanoparticles or microparticles. The resulting nanoparticles or microparticles are washed with water and dried overnight in a lyophilizer. Micro- or nanoparticles with different sizes and morphologies can be obtained by this method.

Pharmaceutical compositions which contain labile polymers, such as certain polyanhydrides, may degrade during the fabrication process due to the presence of water. For these polymers, methods which are performed in completely or substantially anhydrous organic solvents can be used to make the particles.

Solvent removal can also be used to prepare particles from a compound that is hydrolytically unstable. In this method, the compound (or polymer matrix and one or more compounds) is dispersed or dissolved in a volatile organic solvent such as methylene chloride.

This mixture is then suspended by stirring in an organic oil (such as silicon oil) to form an emulsion. Solid particles form from the emulsion, which can subsequently be isolated from the supernatant. The external morphology of spheres produced with this technique is highly dependent on the identity of the drug.

In certain embodiments, compounds of Formulas I, II or III, including levcromakalim, or a pharmaceutically acceptable salt of the present invention described herein is administered to a patient in need thereof as particles formed by solvent removal. In another embodiment the present invention provides particles formed by solvent removal comprising a compound of the present invention and one or more pharmaceutically acceptable excipients as defined herein. In another embodiment the particles formed by solvent removal comprise a compound of the present invention and an additional therapeutic agent. In a further embodiment the particles formed by solvent removal comprise a compound of the present invention, an additional therapeutic agent, and one or more pharmaceutically acceptable excipients. In another embodiment any of the described particles formed by solvent removal can be formulated into a tablet and then coated to form a coated tablet. In an alternative embodiment the particles formed by solvent removal are formulated into a tablet, but the tablet is uncoated.

In certain embodiments, the particles are derived by spray drying. In this method, a compound (or polymer matrix and one or more compounds) is dissolved in an organic solvent such as methylene chloride. The solution is pumped through a micronizing nozzle driven by a flow of compressed gas, and the resulting aerosol is suspended in a heated cyclone of air, allowing the solvent to evaporate from the micro droplets, forming particles. Microparticles and nanoparticles can be obtained using this method.

In other embodiments, compounds of Formulas I, II or III, including levcromakalim, or a pharmaceutically acceptable salt of the present invention described herein is administered to a patient in need thereof as a spray dried dispersion (SDD). In another embodiment the present invention provides a spray dried dispersion (SDD) comprising a compound of the present invention and one or more pharmaceutically acceptable excipients as defined herein. In another embodiment the SDD comprises a compound of the present invention and an additional therapeutic agent. In a further embodiment the SDD comprises a compound of the present invention, an additional therapeutic agent, and one or more pharmaceutically acceptable excipients. In another embodiment any of the described spray dried dispersions can be coated to form a coated tablet. In an alternative embodiment the spray dried dispersion is formulated into a tablet but is uncoated.

Particles can be formed from the active compound as described herein using a phase inversion method. In this method, the compound (or polymer matrix and one or more active compounds) is dissolved in a suitable solvent, and the solution is poured into a strong non-solvent for the compound to spontaneously produce, under favorable conditions, microparticles or nanoparticles. The method can be used to produce nanoparticles in a wide range of sizes, including, for example, from nanoparticles to microparticles, typically possessing a narrow particle size distribution.

In some embodiments, an extended release formulation of a compound of Formulas I, II or III, including levcromakalim, or a pharmaceutically acceptable salt of the present invention described herein is administered to a patient in need thereof as particles formed by phase inversion.

In another embodiment the present invention provides particles formed by phase inversion comprising a compound of the present invention and one or more pharmaceutically acceptable excipients as defined herein. In another embodiment the particles formed by phase inversion comprise a compound of the present invention and an additional therapeutic agent. In a further embodiment the particles formed by phase inversion comprise a compound of the present invention, an additional therapeutic agent, and one or more pharmaceutically acceptable excipients. In another embodiment any of the described particles formed by phase inversion can be formulated into a tablet and then coated to form a coated tablet. In an alternative embodiment the particles formed by phase inversion are formulated into a tablet, but the tablet is uncoated.

Techniques for particle formation using coacervation are known in the art, for example, as described in GB-B-929 406; GB-B-929 40 1; and U.S. Pat. Nos. 3,266,987, 4,794,000, and 4,460,563. Coacervation involves the separation of a compound (or polymer matrix and one or more compounds) solution into two immiscible liquid phases. One phase is a dense coacervate phase, which contains a high concentration of the compound, while the second phase contains a low concentration of the compound. Within the dense coacervate phase, the compound forms nanoscale or microscale droplets, which harden into particles. Coacervation may be induced by a temperature change, addition of a non-solvent or addition of a micro-salt (simple coacervation), or by the addition of another polymer thereby forming an interpolymer complex (complex coacervation).

In one embodiment compounds of Formulas I, II or III, including levcromakalim, or a pharmaceutically acceptable salt of the present invention described herein is administered to a patient in need thereof as extended release particles formed by coacervation. In another embodiment the present invention provides particles formed by coacervation comprising a compound of the present invention and one or more pharmaceutically acceptable excipients as defined herein. In another embodiment the particles formed by coacervation comprise a compound of the present invention and an additional therapeutic agent. In a further embodiment the particles formed by coacervation comprise a compound of the present invention, an additional therapeutic agent, and one or more pharmaceutically acceptable excipients. In another embodiment any of the described particles formed by coacervation can be formulated into a tablet and then coated to form a coated tablet. In an alternative embodiment the particles formed by coacervation are formulated into a tablet, but the tablet is uncoated.

Methods for very low temperature casting of controlled release microspheres are described in U.S. Pat. No. 5,019,400 to Gombotz et al. In this method, the compound is dissolved in a solvent. The mixture is then atomized into a vessel containing a liquid non solvent at a temperature below the freezing point of the drug solution which freezes the compound droplets. As the droplets and non-solvent for the compound are warmed, the solvent in the droplets thaws and is extracted into the non-solvent, hardening the microspheres.

In one embodiment, a selected compound of Formulas I, II or III, including levcromakalim, or a pharmaceutically acceptable salt of the present invention described herein is administered to a patient in need thereof as particles formed by low temperature casting. In another embodiment the present invention provides particles formed by low temperature casting comprising a compound of the present invention and one or more pharmaceutically acceptable excipients as defined herein. In another embodiment the particles formed by low temperature casting comprise a compound of the present invention and an additional therapeutic agent. In a further embodiment the particles formed by low temperature casting comprise a compound of the present invention, an additional therapeutic agent, and one or more pharmaceutically acceptable excipients. In another embodiment any of the described particles formed by low temperature casting can be formulated into a tablet and then coated to form a coated tablet. In an alternative embodiment the particles formed by low temperature casting are formulated into a tablet, but the tablet is uncoated.

In one aspect of the present invention, a selected compound of Formulas I, II or III, including levcromakalim, or a pharmaceutically acceptable salt of the present invention described herein is incorporated into a nanoparticle, e.g., for convenience of delivery and/or extended release delivery. The use of materials in nanoscale provides one the ability to modify fundamental physical properties such as solubility, diffusivity, blood circulation half-life, drug release characteristics, and/or immunogenicity. A number of nanoparticle-based therapeutic and diagnostic agents have been demonstrated to be useful for the treatment of cancer, diabetes, pain, asthma, allergy, and infections. These nanoscale agents may provide more effective and/or more convenient routes of administration, lower therapeutic toxicity, extend the product life cycle, and ultimately reduce health-care costs. As therapeutic delivery systems, nanoparticles can allow targeted delivery and controlled release.

In addition, nanoparticle-based compound delivery can be used to release compounds at a sustained rate and thus lower the frequency of administration, deliver drugs in a targeted manner to minimize systemic side effects, or deliver two or more drugs simultaneously for combination therapy to generate a synergistic effect and suppress drug resistance. A number of nanotechnology-based therapeutic products have been approved for clinical use. Among these products, liposomal drugs and polymer-based conjugates account for a large proportion of the products. See, Zhang, L., et al., Nanoparticles in Medicine: Therapeutic Applications and Developments, Clin. Pharm. and Ther., 83(5):761-769, 2008.

Methods for producing nanoparticles are known in the art. For example, see Muller, R. H., et al., Solid lipid nanoparticles (SLN) for controlled drug delivery—a review of the state of the art, Eur. H. Pharm. Biopharm., 50:161-177, 2000; U.S. Pat. No. 8,691,750 to Consien et al.; WO 2012/145801 to Kanwar. U.S. Pat. No. 8,580,311 to Armes, S. et al.; Petros, R. A. and DeSimone, J. M., Strategies in the design of nanoparticles for therapeutic applications, Nature Reviews/Drug Discovery, vol. 9:615-627, 2010; U.S. Pat. Nos. 8,465,775; 8,444,899; 8,420,124; 8,263,129; 8,158,728; 8,268,446; Pellegrino et al., 2005, Small, 1:48; Murray et al., 2000, Ann. Rev. Mat. Sci., 30:545; and Trindade et al., 2001, Chem. Mat., 13:3843; all incorporated herein by reference. Additional methods have been described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755; U.S. Pat. Nos. 5,578,325 and 6,007,845; P. Paolicelli et al., “Surface-modified PLGA-based Nanoparticles that can Efficiently Associate and Deliver Virus-like Particles” Nanomedicine. 5(6):843-853 (2010)), U.S. Pat. No. 5,543,158 to Gref et al., or WO publication WO2009/051837 by Von Andrian et al. Zauner et al., 1998, Adv. Drug Del. Rev., 30:97; and Kabanov et al., 1995, Bioconjugate Chem., 6:7; (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372; Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-L-lysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633; U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181; Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732; C. Astete et al., “Synthesis and characterization of PLGA nanoparticles” J. Biomater. Sci. Polymer Edn, Vol. 17, No. 3, pp. 247-289 (2006); K. Avgoustakis “Pegylated Poly(Lactide) and Poly(Lactide-Co-Glycolide) Nanoparticles: Preparation, Properties and Possible Applications in Drug Delivery” Current Drug Delivery 1:321-333 (2004); C. Reis et al., “Nanoencapsulation I. Methods for preparation of drug-loaded polymeric nanoparticles” Nanomedicine 2:8-21 (2006); P. Paolicelli et al., “Surface-modified PLGA-based Nanoparticles that can Efficiently Associate and Deliver Virus-like Particles” Nanomedicine. 5(6):843-853 (2010); U.S. Pat. No. 6,632,671 to Unger Oct. 14, 2003, all incorporated herein by reference.

In some aspects, the polymeric particle is between about 0.1 nm to about 10000 nm, between about 1 nm to about 1000 nm, between about 10 nm and 1000 nm, between about 1 and 100 nm, between about 1 and 10 nm, between about 1 and 50 nm, between about 100 nm and 800 nm, between about 400 nm and 600 nm, or about 500 nm. In one embodiment, the micro-particles are no more than about 0.1 nm, 0.5 nm, 1.0 nm, 5.0 nm, 10 nm, 25 nm, 50 nm, 75 nm, 100 nm, 150 nm, 200 nm, 250 nm, 300 nm, 400 nm, 450 nm, 500 nm, 550 nm, 600 nm, 650 nm, 700 nm, 750 nm, 800 nm, 850 nm, 900 nm, 950 nm, 1000 nm, 1250 nm, 1500 nm, 1750 nm, or 2000 nm.

In some embodiments, a compound described herein may be covalently coupled to a polymer used in the nanoparticle, for example a PLGA particle, PLA particle, PGA, or other polymer that covalently binds to the hydroxyl in the compound of Formula I, II or III. Methods for covalent binding of active compounds to biodegradable polymers are well known and published.

### Method of Administration

Extended-release formulations of a selected compound of Formulas I, II or III, including levcromakalim, or a pharmaceutically acceptable salt of the present invention described herein can be administered in an effective amount to a host, typically a human, in need thereof for any of the indications described herein. The extended-release formulation can be provided without a carrier, but they are more typically administered as a pharmaceutical composition that includes an effective amount for a host, typically a human, in need of such treatment an extended-release formulation of a compound of Formulas I, II or III, including levcromakalim, or a pharmaceutically acceptable salt thereof in a pharmaceutically acceptable carrier. Thus, in one embodiment, the disclosure provides pharmaceutical compositions comprising an effective amount of an extended-release formulation of a compound or a pharmaceutically acceptable salt of Formula I-III, including levcromakalim, with at least one pharmaceutically acceptable carrier for any of the uses described herein. The pharmaceutical composition may contain a compound or salt thereof as the only active agent, or, in an alternative embodiment, the compound or salt thereof and at least one additional active agent.

The exact amount of the active compound in the extended-release formulation or pharmaceutical composition described herein to be delivered to the host, typically a human, in need thereof will be determined by the health care provider to achieve the desired clinical benefit.

The pharmaceutical compositions contemplated here typically include a carrier, as described further below. Carriers must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound. Representative carriers include solvents, diluents, pH modifying agents, preservatives, antioxidants, suspending agents, wetting agents, viscosity agents, tonicity agents, stabilizing agents, and combinations thereof. In some embodiments, the carrier is an aqueous carrier.

One or more viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextrin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount of buffer necessary to maintain a pH suitable for the selected administration. Suitable buffers are well known by those skilled in the art. Some examples of useful buffers are acetate, borate, carbonate, citrate, and phosphate buffers.

An extended-release formulation of a compound of Formulas I, II or III, including levcromakalim, or its pharmaceutically acceptable salt of the present invention described herein can be provided in any dosage strength that achieves the desired results and also depends on the route of administration. In certain illustrative non-limiting embodiments, the pharmaceutical composition is in a dosage form that contains from about 0.01 mg to about 2000 mg, from up to about 1, 5 or 10 mg to about 1000 mg, from up to about 100 mg to about 800 mg, or from up to about 200 mg to about 600 mg of the active compound and optionally from up to about 0.1 mg to about 2000 mg, from up to about 10 mg to about 1000 mg, from up to about 100 mg to up to about 800 mg, or from about 200 mg to about 600 mg of an additional active agent in a unit dosage form. Examples are dosage forms with at least about 0.1, 0.2, 0.25, 0.5, 1, 1.5, 2, 2.5, 3, 3.5, 4, 4.5, 5, 10, 15, 20, 25, 50, 75, 100, 150, 200, 250, 300, 350, 400, 450, 500, 550, 600, 650, 700, 750, 800, 900, 1000, 1100, 1200, 1250, 1300, 1400, 1500, or 1600 mg of active compound or its salt. In certain embodiments, the dosage form has at least about 0.1 mg, 0.25 mg, 0.5 mg, 0.75 mg, 1 mg, 5 mg, 10 mg, 25 mg, 50 mg, 75 mg, 100 mg, 200 mg, 400 mg, 500 mg, 600 mg, 1000 mg, 1200 mg, or 1600 mg of active compound or its salt. The amount of active compound in the dosage form is calculated without reference to the salt.

In alternative embodiments, the pharmaceutical composition is in a dosage form that contains from about 0.005 mg to about 5 mg, from about 0.003 mg to about 3 mg, from about 0.001 mg to about 1 mg, from about 0.05 mg to about 0.5 mg, from about 0.03 mg to about 0.3 mg, or from about 0.01 mg to about 0.1 mg, or from about 0.01 to about 0.05 mg of a compound of the extended-release formulation of a compound or a pharmaceutically acceptable salt thereof of Formula I-III, including levcromakalim. In one embodiment, the dosage form has at least about 0.01 mg, 0.02 mg, 0.025 mg, or 0.05 mg of active compound or its salt.

As non-limiting embodiments, a therapeutically effective amount of the present compounds in a pharmaceutical dosage form may range, for example, from about 0.001 mg/kg to about 100 mg/kg per day or more. An extended-release formulation of a compound of Formula I, Formula IL, or Formula III or a pharmaceutically acceptable salt thereof, may for example in non-limiting embodiments, be administered in amounts ranging from about 0.1 mg/kg to about 35 mg/kg per day of the patient, depending upon the pharmacokinetics of the agent in the patient. In an alternative embodiment, an extended-release formulation of a compound of Formula I, Formula IL, or Formula III or a pharmaceutically acceptable salt thereof, including levcromakalim, may be administered in amounts ranging from about 0.01 mg/kg to about 3.5 mg/kg per day of the patient, depending upon the pharmacokinetics of the agent in the patient.

In certain embodiments, an extended-release formulation of a compound of Formula I, Formula II, or Formula III or a pharmaceutically acceptable salt thereof, including levcromakalim, is administered for at least about one day, two days, three days, four days, five days, six days, seven days, eight days, nine days, ten days, two weeks, three weeks, one month, at least two months, at least three months, at least four months, at least five months, at least six months or more, including indefinitely during therapy. In certain embodiments, an extended-release formulation of a compound of Formula I, Formula IL, or Formula III or a pharmaceutically acceptable salt thereof, including levcromakalim, is administered once, twice, three, or more times a day.

Non-limiting examples of buffers, with or without additional excipients or other additives, that can be used as a pharmaceutically acceptable formulation for an appropriate indication as described herein include, for example (with illustrative, but not limiting concentrations and pHs), Acetate Buffer (0.1 M, pH 5.0); BES-Buffered Saline (2×) (0.05 M, pH 6.95); Bicine (1 M, pH 8.26); CAPS (1 M, pH 10.4); CHES (1 M, pH 9.5); Citrate Buffer (0.1 M, pH 6.0); Citrate-Phosphate Buffer (0.15 M, pH 5.0); Diethanolamine (1 M, pH 9.8); EBSS (magnesium, calcium, phenol red) (pH 7.0); Glycine-HCl Buffer (0.1 M, pH 3.0); Glycine-Sodium Hydroxide Buffer (0.08 M, pH 10); HBSS (Hank's Balanced Salt Solution); HEPPSO (1 M, pH 7.85); HHBS (Hank's Buffer with Hepes); Hydrochloric Acid-Potassium Chloride Buffer (0.1 M, pH 2.0); Imidazole-HCl Buffer (0.05 M, pH 7.0); MES (0.5 M, pH 6); MOPS Buffer (10×) (0.2 M, pH 7); PBS (Phosphate Buffered Saline) (1×, pH 7.4)); Sodium Borate Buffer (1 M, pH 8.5); TAE (1 M, pH 8.6); TAE Buffer (50×) (0.04 M, pH 8.5); TBS (1 M, pH 7.4); TE Buffer 10×; Tricine (1 M, pH 8.05); Tris Buffer (1 M, pH 7.2); Acetate Buffer (pH 3.6 to 5.6); Carbonate-Bicarbonate Buffer (pH 9.2 to 10.6); Citrate Buffer (pH 3.0 to 6.2); Phosphate Buffer (pH 5.8 to 8.0); Potassium Phosphate (pH 5.8 to 8.0); and, Trizma® Buffer (pH 7.0 to 9.2).

Formulations for ocular, topical, enteric and parenteral delivery are described in more detail below.

### Ocular Delivery

When used for ocular treatment, an effective amount of an extended-release formulation of a compound of Formula I, II or III, including levcromakalim, or its pharmaceutically acceptable salt of the present invention herein can be administered, for example, as a topical formulation, such as a solution, suspension, or emulsion. The topical formulation typically comprises a pharmaceutically acceptable carrier, which can be an aqueous or non-aqueous carrier.

Examples of aqueous carriers include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), simulated body fluids including simulated aqueous humor, tears, plasma platelet concentrate and tissue culture medium or an aqueous solution or suspension comprising an organic solvent. Pharmaceutical formulations for ocular administration are preferably in the form of a sterile aqueous solution. Acceptable solutions include, for example, water, Ringer's solution, phosphate buffered saline (PBS), citrate buffered saline, and isotonic sodium chloride solutions. The formulation may also be a sterile solution, suspension, or emulsion in a non-toxic diluent or solvent such as 1,3-butanediol. In one embodiment, the carrier is PBS. In one embodiment, the carrier is citrate-buffer, including citrate buffered saline. Further examples of buffers that can be used in a pharmaceutically acceptable ocular formulation for an appropriate indication are described above.

Suitable non-aqueous pharmaceutically acceptable carriers include but are not limited to oleoyl polyethyleneglycol gylcerides, linoleoyl polyethyleneglycol gylcerides, lauroyl polyethyleneglycol gylcerides, hydrocarbon vehicles like liquid paraffin (Paraffinum liquidum, mineral oil), light liquid paraffin (low viscosity paraffin, Paraffinum perliquidum, light mineral oil), soft paraffin (vaseline), hard paraffin, vegetable fatty oils like castor oil, peanut oil or sesame oil, synthetic fatty oils like middle chain trigylcerides (MCT, triglycerides with saturated fatty acids, preferably octanoic and decanoic acid), isopropyl myristate, caprylocaproyl macrogol-8 glyceride, caprylocaproyl polyoxyl-8 glycerides, wool alcohols like cetylstearylalcohols, wool fat, glycerol, propylene glycol, propylene glycol diesters of caprylic/capric acid, polyethyleneglycols (PEG), semifluorinated alkanes (e.g. as described in WO 2011/113855) or a mixture of thereof. Preferably non-aqueous pharmaceutically acceptable vehicles used for the solution are hydrophobic.

Pharmaceutically acceptable excipients used in the topical ophthalmological pharmaceutical composition according to the present invention include but are not limited to stabilizers, surfactants, polymer-based carriers like gelling agents, organic co-solvents, pH active components, osmotic active components and preservatives.

Surfactants used in the topical ophthalmological pharmaceutical composition according to the present invention include but are not limited to lipids such as phospholipids, phosphatidylcholines, lecithin, cardiolipins, fatty acids, phosphatidylethanolamines, phosphatides, tyloxapol, polyethylenglycols and derivatives like PEG 400, PEG 1500, PEG 2000, poloxamer 407, poloxamer 188, polysorbate 80, polysorbate 20, sorbitan laurate, sorbitan stearate, sorbitan palmitate or a mixture thereof, preferably polysorbate 80. Suitable polymer base carriers like gelling agents used in the topical ophthalmological pharmaceutical composition according to the present invention include but are not limited to cellulose, hydroxypropylmethylcellulose (HPMC), hydroxypropylcellulose (HPC), carboxymethyl cellulose (CMC), methylcellulose (MC), hydroxyethylcellulose (HEC), amylase and derivatives, amylopectins and derivatives, dextran and derivatives, polyvinylpyrrolidone (PVP), polyvinyl alcohol (PVA), and acrylic polymers such as derivatives of polyacrylic or polymethacrylic acid like HEMA, carbopol and derivatives of the before mentioned or a mixture thereof.

A suitable pH active component such as a buffering agent or pH-adjusting agent used in the pharmaceutical composition according to the invention include but are not limited to acetate, borate, carbonate, citrate, and phosphate buffers, including disodium phosphate, monosodium phosphate, boric acid, sodium borate, sodium citrate, hydrochloric acid, sodium hydroxide. The pH active components are chosen based on the target pH for the composition which generally ranges from pH 4-9. In certain embodiments, the extended-release formulation comprising a compound or pharmaceutically acceptable salt thereof of Formula I-III has a pH approximately between 5 and 8, between 5.5 and 7.4, between 6 and 7.5, or between 6.5 and 7. In one embodiment, the formulation comprises a citrate buffer at a pH around 6.5 to 7. In another embodiment, the formulation comprises a phosphate buffer at a pH around 6.5 to 7. Suitable osmotic active components used in the pharmaceutical composition according to the invention include but are not limited to sodium chloride, mannitol and glycerol.

Organic co-solvents used in the pharmaceutical composition according to the invention include but are not limited to ethylene glycol, propylene glycol, N-methyl pyrrolidone, 2-pyrrolidone, 3-pyrrolidinol, 1,4-butanediol, dimethylglycol monomethylether, diethyleneglycol monomethylether, solketal, glycerol, polyethylene glycol, polypropylene glycol.

Preservatives used in the pharmaceutical composition according to the invention include but are not limited to benzalkonium chloride, alkyldimethylbenzylammonium chloride, cetrimide, cetylpyridinium chloride, benzododecinium bromide, benzethonium chloride, thiomersal, chlorobutanol, benzyl alcohol, phenoxethanol, phenylethyl alcohol, sorbic acid, methyl and propyl parabens, chlorhexidine digluconate, EDTA or mixtures thereof.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextrin, polysaccharides, polyacrylamide, polyvinyl alcohol (including partially hydrolyzed polyvinyl acetate), polyvinyl acetate, derivatives thereof and mixtures thereof. In one embodiment, the viscosity agent is hyaluronic acid, and the hyaluronic acid is cross-linked. In one embodiment, the viscosity agent is hyaluronic acid and hyaluronic acid is linear.

The topical dosage form can be administered, for example, once a day (q.d.), twice a day (b.i.d.), three times a day (t.i.d.), four times a day (q.i.d.), once every other day (Q2d), once every third day (Q3d), as needed, or any dosage schedule that provides treatment of a disorder described herein. Alternatively, the extended release formulation can be prepared for long term delivery, such as every 1, 2, 3, 4, 5, 6, 7, 8, 9, 10, 11 or 12 weeks or more, or every 1, 2, 3, 4, 5 or 6 months or more or less.

In certain nonlimiting embodiments, the pharmaceutical composition is in an ocular dosage form that contains from about 0.005 mg to about 5 mg, from about 0.003 mg to about 3 mg, from about 0.001 mg to about 1 mg, from about 0.05 mg to about 0.5 mg, from about 0.03 mg to about 0.3 mg, or from about 0.01 mg to about 0.1 mg, or from about 0.01 to about 0.05 mg of an extended-release formulation of a compound or a pharmaceutically acceptable salt thereof of Formula I-II, including levcromakalim.

In certain embodiments, the ocular solution comprises approximately 0.1% to 5.0% of an extended-release formulation of a compound of Formula I-III or a pharmaceutically acceptable salt thereof as measured in mg/mL. In certain embodiments, the ocular solution comprises approximately 5% to 30% of a compound of Formula I-III as measured in mg/mL. In certain embodiments, the solution comprises approximately 0.2% to 4.5%, 0.3% to 3.0%, 0.4% to 2.0%, or 0.5% to 1.5% of a compound of Formula I-III as measured in mg/mL. In certain embodiments, the solution comprises at least 10%, at least 8%, at least 5%, at least 4%, at least 3%, at least 2%, at least 1%, at least 0.9%, at least 0.7%, at least 0.5%, at least 0.3%, or at least 0.1% of an extended-release formulation of a compound of Formula I-III. In other embodiments, the solution comprises at least 30%, at least 25%, at least 20%, or at least 15% of a compound of Formula I-III. In certain embodiments, the solution comprises approximately 0.2%, 0.4%, or 0.8% of an extended-release formulation of a compound of Formula I-III or salts thereof, including levcromakalim. In certain embodiments, the solution comprises at least approximately 0.5%, 1%, or 2% of an extended-release formulation of a compound of Formula I-III or salts thereof.

In alternative embodiments, the ocular solution comprises approximately 0.01% to 5.0% of an extended-release formulation of a compound of Formula I-III or a pharmaceutically acceptable salt thereof, including levcromakalim, as measured in mg/mL. In certain embodiments, the solution comprises approximately 0.01% to 3%, 0.01% to 1.0%, 0.01% to 0.5%, 0.01% to 0.1%, 0.01% to 0.08%, or 0.01% to 0.05% of a compound of Formula I-II as measured in mg/mL.

In other embodiments, the solution has a concentration of an extended-release formulation of a compound of Formula I-III or a pharmaceutically acceptable salt thereof, including levcromakalim, ranging from about 2.5 mM to 500 mM. In certain embodiments, the concentration is not greater than about 550 mM, 500 mM, 450 mM, 400 mM, 350 mM, 300 mM, 250 mM, 200 mM, 150 mM, 100 mM, 50 mM, 45 mM, 40 mM, 35 mM, 30 mM, 25 mM, 20 mM, 15 mM, 10 mM, 8 mM, 6 mM, 5 mM, 4 mM, 3 mM, 2.5 mM, 2.0 mM, 1.5 mM, or 1.0 mM.

In alternative embodiments, the solution has a concentration of an extended-release formulation of a compound of Formula I-III or a pharmaceutically acceptable salt thereof, including levcromakalim, ranging from about 0.1 mM to 2.5 mM. In certain embodiments, the concentration is not greater than about 1.0 mM, 0.9 mM, 0.8 mM, 0.7 mM, 0.6 mM, 0.5 mM, 0.4 mM, 0.3 mM, 0.2 mM, or 0.1 mM.

In certain embodiments, the concentration of an extended-release formulation of a compound of Formula I-III or a pharmaceutically acceptable salt thereof, including levcromakalim, is in the range of approximately 0.2%-2% (equivalent to a 5 mM to 52 mM solution). In certain embodiments, the concentration is at least 0.2% (equivalent to 5M), at least 0.4% (equivalent to 10 mM), at least 0.5% (equivalent to 12.5 mM), at least 0.8% (equivalent to 20 mM), at least 1% (equivalent to approximately 25 mM), or at least 2% (equivalent to approximately 50 mM).

In alternative embodiments, the concentration of an extended-release formulation of a compound of Formula I-III or a pharmaceutically acceptable salt thereof, including levcromakalim, is in the range of approximately 0.02%-0.2%. In one embodiment, the concentration is at least 0.02%, at least 0.04%, at least 0.05%, at least 0.08%, at least 0.1%, or at least 0.2%.

An extended-release formulation of a compound or a pharmaceutically acceptable salt thereof of Formula I-III, including levcromakalim, can also be used for ocular therapy using an alternative route: intravitreal, intrastromal, intracameral, sub-tenon, sub-retinal, retro-bulbar, peribulbar, suprachoroidal, subchoroidal, choroidal, conjunctival, subconjunctival, episcleral, periocular, transscleral, posterior juxtascleral, circumcorneal, or tear duct injections, or through a mucus, mucin, or a mucosal barrier, in an immediate or controlled release fashion or via an ocular device, or injection. In one embodiment, the ocular device is a contact lens that releases the extended-release formulation of a compound or a pharmaceutically acceptable salt thereof of Formula I-II, including levcromakalim.

In one embodiment, an extended-release formulation of a compound or a pharmaceutically acceptable salt thereof of Formula I-III, including levcromakalim, is administered via suprachoroidal injection. Suprachoroidal delivery is described in U.S. Pat. Nos. 9,636,332; 9,539,139; 10,188,550; 9,956,114; 8,197,435; 7,918,814 and PCT Applications WO 2012/051575; WO 2015/095772; WO 2018/031913; WO 2017/192565; WO 2017/190142; WO 2017/120601;
and WO 2017/120600.

A device for minimally invasive delivery of drugs to the suprachoroidal space may comprise a needle for injection of drugs or drug containing materials directly to the suprachoroidal space. The device may also comprise elements to advance the needle through the conjunctiva and sclera tissues to or just adjacent to the suprachoroidal space without perforation or trauma to the inner choroid layer. The position of the leading tip of the delivery device may be confirmed by non-invasive imaging such as ultrasound or optical coherence tomography, external depth markers or stops on the tissue-contacting portion of the device, depth or location sensors incorporated into the device or a combination of such sensors. For example, the delivery device may incorporate a sensor at the leading tip such as a light pipe or ultrasound sensor to determine depth and the location of the choroid or a pressure transducer to determine a change in local fluid pressure from entering the suprachoroidal space. In certain embodiments, the suprachoroidal injection is conducted with a thin- or regular-walled needle of 26-, 27-, 28-, 29- or 30-gauge. In alternative embodiments, the suprachoroidal injection is conducted with a thin- or regular-walled needle of 31, 32, or 33-gauge. In further alternative embodiments, the suprachoroidal injection is conducted with a thin- or regular-walled needle of 34-gauge or smaller gauge.

Additional non-limiting examples of how to deliver the active compounds are provided in WO/2015/085251 titled “Intracameral Implant for Treatment of an Ocular Condition” (Envisia Therapeutics, Inc.); WO/2011/008737 titled “Engineered Aerosol Particles, and Associated Methods”, WO/2013/082111 titled “Geometrically Engineered Particles and Methods for Modulating Macrophage or Immune Responses”, WO/2009/132265 titled “Degradable compounds and methods of use thereof, particularly with particle replication in non-wetting templates”, WO/2010/099321 titled “Interventional drug delivery system and associated methods”, WO/2008/100304 titled “Polymer particle composite having high fidelity order, size, and shape particles”, WO/2007/024323 titled “Nanoparticle fabrication methods, systems, and materials” (Liquidia Technologies, Inc. and the University of North Carolina at Chapel Hill); WO/2010/009087 titled “Iontophoretic Delivery of a Controlled-Release Formulation in the Eye”, (Liquidia Technologies, Inc. and Eyegate Pharmaceuticals, Inc.) and WO/2009/132206 titled “Compositions and Methods for Intracellular Delivery and Release of Cargo”, WO/2007/133808 titled “Nano-particles for cosmetic applications”, WO/2007/056561 titled “Medical device, materials, and methods”, WO/2010/065748 titled “Method for producing patterned materials”, WO/2007/081876 titled “Nanostructured surfaces for biomedical/biomaterial applications and processes thereof” (Liquidia Technologies, Inc.).

In one embodiment, an extended-release formulation of a compound of Formula I-Formula III is stored as a depot in tissues and then slowly released over time where it is converted to levcromakalim to induce an IOP-lowering effect. In one embodiment, an extended-release formulation of a compound of Formula I-Formula III is stored in the trabecular meshwork and then slowly released to the proximal distal outflow pathway. In one embodiment, the return to baseline IOP following a dosage form of an extended-release formulation of a compound of Formula I-Formula III in a host in need thereof, including a human, is at least about 12 hours, at least about 24 hours, at least about 36 hours, at least about 48 hours, at least about 60 hours, or at least about 72 hours.

### Topical Skin or Transdermal Delivery

Administration of an extended-release formulation of a compound or a pharmaceutically acceptable salt of Formula I-III, including levcromakalim, may also include topical or transdermal administration. Pharmaceutical compositions suitable for topical application to the skin may take the form of a gel, ointment, cream, lotion, paste, spray, aerosol, or oil, and may optionally include petroleum jelly, lanoline, polyethylene glycol, alcohol, or a combination thereof.

Pharmaceutical compositions suitable for transdermal administration may be presented as discrete patches adapted to remain in intimate contact with the epidermis of the recipient for a prolonged period of time. Pharmaceutical compositions suitable for transdermal administration may also be delivered by iontophoresis (see, for example, Pharmaceutical Research 3 (6):318 (1986)) and typically take the form of an optionally buffered aqueous solution of the active compound. In one embodiment, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

A wide variety of skin care active and inactive ingredients may be advantageously combined with the present compounds in accordance with the present invention, including, but not limited to, conditioning agents, skin protectants, other antioxidants, UV absorbing agents, sunscreen actives, cleansing agents, viscosity modifying agents, film formers, emollients, surfactants, solubilizing agents, preservatives, fragrance, chelating agents, foaming or antifoaming agents, opacifying agents, stabilizing agents, pH adjustors, absorbents, anti-caking agents, slip modifiers, various solvents, solubilizing agents, denaturants, abrasives, bulking agents, emulsion stabilizing agents, suspending agents, colorants, binders, conditioning agent-emollients, surfactant emulsifying agents, biological products, anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sebum stimulators, sebum inhibitors, humectants, and/or combinations thereof.

Conditioning agents may generally be used to improve the appearance and/or feel of the skin upon and after topical application via moisturization, hydration, plasticization, lubrication, and occlusion, or a combination thereof. Non-limiting examples of the conditioning component include, but are not limited to, mineral oil, petrolatum, C₇-C₄₀ branched chain hydrocarbons, C₁-C₃₀ alcohol esters of C₁-C₃₀carboxylic acids, C₁-C₃₀ alcohol esters of C₂-C₃₀ dicarboxylic acids, monoglycerides of C₁-C₃₀carboxylic acids, diglycerides of C₁-C₃₀ carboxylic acids, triglycerides of C₁-C₃₀ carboxylic acids, ethylene glycol monoesters of C₁-C₃₀ carboxylic acids, ethylene glycol diesters of C₁-C₃₀carboxylic acids, propylene glycol monoesters of C₁-C₃₀ carboxylic acids, propylene glycol diesters of C₁-C₃₀ carboxylic acids, C₁-C₃₀ carboxylic acid monoesters and polyesters of sugars, polydialkylsiloxanes, polydiarylsiloxanes, polyalkarylsiloxanes, cylcomethicones having 3 to 9 silicon atoms, vegetable oils, hydrogenated vegetable oils, polypropylene glycol C₄-C₂₀ alkyl ethers, di C₈-C₃₀ alkyl ethers, and mixtures thereof. Non-limiting examples of straight and branched chain hydrocarbons having from about 7 to about 40 carbon atoms include, but are not limited to, dodecane, isododecane, squalane, cholesterol, hydrogenated olyisobutylene, docosane hexadecane, isohexadecane, C₇-C₄₀ isoparaffins, monoglycerides of C₁-C₃₀ carboxylic acids, diglycerides of C₁-C₃₀ carboxylic acids, triglycerides of C₁-C₃₀ carboxylic acids, ethylene glycol monoesters of C₁-C₃₀ carboxylic acids, ethylene glycol diesters of C₁-C₃₀ carboxylic acids, propylene glycol monoesters of C₁-C₃₀ carboxylic acids, and propylene glycol diesters of C₁-C₃₀ carboxylic acids, including straight chain, branched chain and aryl carboxylic acids, and propoxylated and ethoxylated derivatives of these materials.

Non-limiting examples of sugars include sucrose, mannitol, trehalose, glucose, arabinose, fucose, mannose, rhamnose, xylose, D-xylose, glucose, fructose, ribose, D-ribose, galactose, dextrose, dextran, lactose, maltodextrin, maltose, glycerol, erythritol, threitol, arabitol, xylitol, ribitol, sorbitol, galactitol, fucitol, iditol, inositol, volemitol, isomalt, maltitol, lactitol, maltotriitol, maltotetraitol, polyglycitol, aspartame, saccharin, stevia, sucralose, acesulfame potassium, advantame, alitame, neotame, and sucralose.

Non-limiting examples of sunscreens which are useful in the compositions include 4-N,N-(2-ethylhexyl)methylaminobenzoic acid ester of 2,4-dihydroxybenzophenone, 4-N,N-(2-ethylhexyl)methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N-(2-ethylhexyl)-methylaminobenzoic acid ester of 4-(2-hydroxyethoxy)dibenzoylmethane, 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, titanium dioxide, zinc oxide, silica, iron oxide, and mixtures thereof. Other useful sunscreens include 4-aminobenzoic acid (PABA), benzylidene camphor, butyl methoxy dibenzoyl methane, diethanolamine p-methoxycinnamate, 5 dioxybenzone, ethyl dihydroxypropyl PABA, glyceryl aminobenzoate, homomenthyl salicylate, isopropyl dibenzoyl methane, lawsone and dihydroxyacetone, menthyl anthranilate, methyl anthranilate, methyl benzylidene camphor, octocrylene, octyl dimethyl PABA, octyl methoxycinnamate, oxybenzone, 2-phenylbenzimidazole-5-sulfonic acid, red petrolatum, sulisobenzone, titanium dioxide, triethanolamine salicylate, zinc oxide, and mixtures thereof.

Exact amounts of sunscreens which can be employed will vary depending upon the sunscreen chosen and the desired Sun Protection Factor (SPF) to be achieved.

Viscosity agents may be added to the topical formulation to increase the viscosity of the composition as desired. Examples of useful viscosity agents include, but are not limited to, water-soluble polyacrylic and hydrophobically modified polyacrylic resins such as Carbopol and Pemulen; starches such as corn starch, potato starch, and tapioca; gums such as guar gum and gum arabic; and cellulose ethers such as hydroxypropyl cellulose, hydroxyethyl cellulose, carboxymethyl cellulose, and the like.

A wide variety of emulsifiers are also useful and include, but are not limited to, sorbitan esters, glyceryl esters, poly glyceryl esters, methyl glucose esters, sucrose esters, ethoxylated fatty alcohols, hydrogenated castor oil ethoxylates, sorbitan ester ethoxylates, polymeric emulsifiers, silicone emulsifiers, glyceryl monoesters, preferably glyceryl monoesters of C₁₆-C₂₂ saturated, unsaturated and branched chain fatty acids such as glyceryl oleate, glyceryl monostearate, glyceryl monopalmitate, glyceryl monobehenate, and mixtures thereof, polyglyceryl esters of C₁₆-C₂₂ saturated, unsaturated and branched chain fatty acids, such as polyglyceryl-4 isostearate, polyglyceryl-3 oleate, diglycerol monooleate, tetraglycerol monooleate and mixtures thereof, methyl glucose esters, preferably methyl glucose esters of C₁₆-C₂₂ saturated, unsaturated and branched chain fatty acids such as methyl glucose dioleate, methyl glucose sesquhsostearate, and mixtures thereof; sucrose fatty acid esters, preferably sucrose esters of C₁₂-C₂₂ saturated, unsaturated and branched chain fatty acids such as sucrose stearate, sucrose laurate, sucrose distearate (e.g., CRODESTA® F10), and mixtures thereof, C₁₂-C₂₂ ethoxylated fatty 5 alcohols such as oleth-2, oleth-3, steareth-2, and mixtures thereof, hydrogenated castor oil ethoxylates such as PEG-7 hydrogenated castor oil; sorbitan ester ethoxylates such as PEG-40 sorbitan peroleate, Polysorbate-80, and mixtures thereof; polymeric emulsifiers such as ethoxylated dodecyl glycol copolymer; and silicone emulsifiers such as laurylmethicone copolyol, cetyldimethicone, dimethicone copolyol, and mixtures thereof.

### Systemic Delivery

In another embodiment, an extended-release formulation of a compound or a pharmaceutically acceptable salt thereof of Formula I-III, including levcromakalim, is administered in an effective amount via any systemic route that achieves the desired effect. Examples are enteral or parenteral administration, including via oral, buccal, sublingual, intravenous, subcutaneous, intramuscular, intrathecal, or intranasal delivery, including a solution, a suspension, emulsion, or a lyophilized powder. In some instances, the composition is distributed or packaged in a liquid form. Alternatively, formulations can be packaged as a solid, obtained, for example by lyophilization of a suitable liquid formulation. The solid can be reconstituted with an appropriate carrier or diluent prior to administration. In one embodiment, the compound is administered vaginally via a suppository, a cream, a gel, a lotion, or an ointment.

Other forms of administration include oral, rectal, sublingual, sublabial, or buccal and typical dosage forms for these routes include a pill, a tablet, a capsule, a solution, a suspension, an emulsion, or a suppository.

In one embodiment, an extended-release formulation of a compound or a pharmaceutically acceptable salt thereof of Formula I-III, including levcromakalim, is administered via the inhaled pulmonary route. Dosage forms for pulmonary drug delivery include propellants, non-aqueous inhalers, dry powder inhalers, and jet or ultrasonic nebulizers.

### Oral Delivery

In one aspect, an extended-release formulation of a compound or a pharmaceutically acceptable salt thereof of Formula I-III, including levcromakalim, is administered orally. The extended-release formulation can be formulated using any desired techniques including formulating the extended-release formulation as a neat chemical (for example a powder, morphic form, amorphous form, or oil), or mixing the extended-release formulation with a pharmaceutically acceptable excipient. The resulting pharmaceutically acceptable composition for oral delivery contains an effective amount of the extended-release formulation or a pharmaceutically acceptable salt thereof and one or more pharmaceutically acceptable excipients.

### Excipients

Pharmaceutically acceptable excipients should be of sufficiently high purity and sufficiently low toxicity to render them suitable for administration to the patient being treated. The excipient can be inert or it can possess pharmaceutical benefits of its own. The amount of excipient employed in conjunction with the compound is sufficient to provide a practical quantity of material for administration per unit dose of the compound. Classes of excipients include, but are not limited to binders, buffering agents, coloring agents, diluents, disintegrants, emulsifiers, fillers, flavorants, glidents, lubricants, pH modifiers, preservatives, stabilizers, surfactants, solubilizers, tableting agents, and wetting agents. Exemplary pharmaceutically acceptable excipients include sugars, starches, celluloses, powdered tragacanth, malt, gelatin, talc, and vegetable oils. Examples of other matrix materials, fillers, or diluents include lactose, mannitol, xylitol, microcrystalline cellulose, calcium diphosphate, and starch. Examples of surface-active agents include sodium lauryl sulfate and polysorbate 80. Examples of drug complexing agents or solubilizers include the polyethylene glycols, caffeine, xanthene, gentisic acid and cyclodextrins. Examples of disintegrants include sodium starch glycolate, sodium alginate, carboxymethyl cellulose sodium, methyl cellulose, colloidal silicon dioxide, and croscarmellose sodium. Examples of binders include methyl cellulose, microcrystalline cellulose, starch, gums, and tragacanth. Examples of lubricants include magnesium stearate and calcium stearate. Examples of pH modifiers include acids such as citric acid, acetic acid, ascorbic acid, lactic acid, aspartic acid, succinic acid, phosphoric acid, and the like; bases such as sodium acetate, potassium acetate, calcium oxide, magnesium oxide, trisodium phosphate, sodium hydroxide, calcium hydroxide, aluminum hydroxide, and the like, and buffers generally comprising mixtures of acids and the salts of said acids. Optionally, other active agents may be included in a pharmaceutical composition, so long as they do not substantially interfere with the activity of the compound of the present invention.

In certain embodiments the excipient is selected from phosphoglyceride; phosphatidylcholine; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohol, polyethylene glycol (PEG); polyoxyethylene-9-lauryl ether; a surface active fatty acid, such as palmitic acid or oleic acid; fatty acid; fatty acid monoglyceride; fatty acid diglyceride; fatty acid amide; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such as sorbitan trioleate; lecithin; lysolecithin; phosphatidylserine; phosphatidylinositol; sphingomyelin; phosphatidylethanolamine (cephalin); cardiolipin; phosphatidic acid; cerebroside; dicetylphosphate; dipalmitoylphosphatidylglycerol; stearylamine; dodecylamine; hexadecyl-amine; acetyl palmitate; glycerol ricinoleate; hexadecyl stearate; isopropyl myristate; tyloxapol; poly(ethylene glycol)5000-phosphatidylethanolamine; poly(ethylene glycol)400-monostearate; phospholipid; synthetic and/or natural detergent having high surfactant properties; deoxycholate; cyclodextrin; chaotropic salt; ion pairing agent; glucose, fructose, galactose, ribose, lactose, sucrose, maltose, trehalose, cellbiose, mannose, xylose, arabinose, glucuronic acid, galacturonic acid, mannuronic acid, glucosamine, galactosamine, and neuramic acid; pullulan, cellulose, microcrystalline cellulose, hydroxypropyl methylcellulose (HPMC), hydroxycellulose (HC), methylcellulose (MC), dextran, cyclodextrin, glycogen, hydroxyethylstarch, carageenan, glycon, amylose, chitosan, N,O-carboxylmethylchitosan, algin and alginic acid, starch, chitin, inulin, konjac, glucomannan, pustulan, heparin, hyaluronic acid, curdlan, and xanthan, mannitol, sorbitol, xylitol, erythritol, maltitol, and lactitol, a pluronic polymer, polyethylene, polycarbonate (e.g. poly(1,3-dioxan-2one)), polyanhydride (e.g. poly(sebacic anhydride)), polypropylfumerate, polyamide (e.g. polycaprolactam), polyacetal, polyether, polyester (e.g., polylactide, polyglycolide, polylactide-co-glycolide, polycaprolactone, polyhydroxyacid (e.g. poly(p-hydroxyalkanoate)), poly(orthoester), polycyanoacrylate, polyvinyl alcohol, polyurethane, polyphosphazene, polyacrylate, polymethacrylate, polyurea, polystyrene, and polyamine, polylysine, polylysine-PEG copolymer, and poly(ethyleneimine), poly(ethylene imine)-PEG copolymer, glycerol monocaprylocaprate, propylene glycol, Vitamin E TPGS (also known as d-α-Tocopheryl polyethylene glycol 1000 succinate), gelatin, titanium dioxide, polyvinylpyrrolidone (PVP), hydroxypropyl methyl cellulose (HPMC), hydroxypropyl cellulose (HPC), methyl cellulose (MC), block copolymers of ethylene oxide and propylene oxide (PEO/PPO), polyethyleneglycol (PEG), sodium carboxymethylcellulose (NaCMC), or hydroxypropylmethyl cellulose acetate succinate (HPMCAS).

### Oral Dosage Forms

Typical dosage forms for oral administration includes a pill, a tablet, a capsule, a gel cap, a solution, a suspension, or an emulsion. The dosage form may also feature compartmentalization. For example, when the dosage form is a pill, tablet, or capsule, it may have different layers of material which have different excipients or different concentrations of excipients. For example, an enteric coated oral tablet may be used to enhance bioavailability of the compounds for an oral route of administration. The enteric coating will be a layer of excipient that allows the tablet to survive stomach acid. The most effective dosage form will depend upon the bioavailability/pharmacokinetic of the particular agent chosen as well as the severity of disease in the patient. Oral dosage forms are particularly preferred, because of ease of administration and prospective favorable patient compliance.

In certain embodiments the oral dosage form contains one or more additional active agents as described herein. In certain embodiments the second active agent is administered separately from the compound of the present invention.

In another embodiment one dosage form may be converted to another to favorably improve the properties. For example, when making a solid pharmaceutically acceptable composition a suitable liquid formulation can be lyophilization. The solid can be reconstituted with an appropriate carrier or diluent prior to administration.

Oral pharmaceutical compositions can contain any amount of active compound that achieves the desired result, for example between 0.1 and 99 weight % (wt. %) of the compound and usually at least about 5 wt. % of the compound. Some embodiments contain at least about 10%, 15%, 20%, 25 wt. % to about 50 wt. % or from about 5 wt. % to about 75 wt. % of the compound.

The oral dosage form can be administered, for example, once a day (q.d.), twice a day (b.i.d.), three times a day (t.i.d.), four times a day (q.i.d.), once every other day (Q2d), once every third day (Q3d), as needed, or any dosage schedule that provides treatment of a disorder described herein.

### General Synthesis of Compounds of the Present Invention and Pharmaceutically Acceptable Salts Thereof.

The described pharmaceutically acceptable salts of the present invention can be prepared according to known methods.

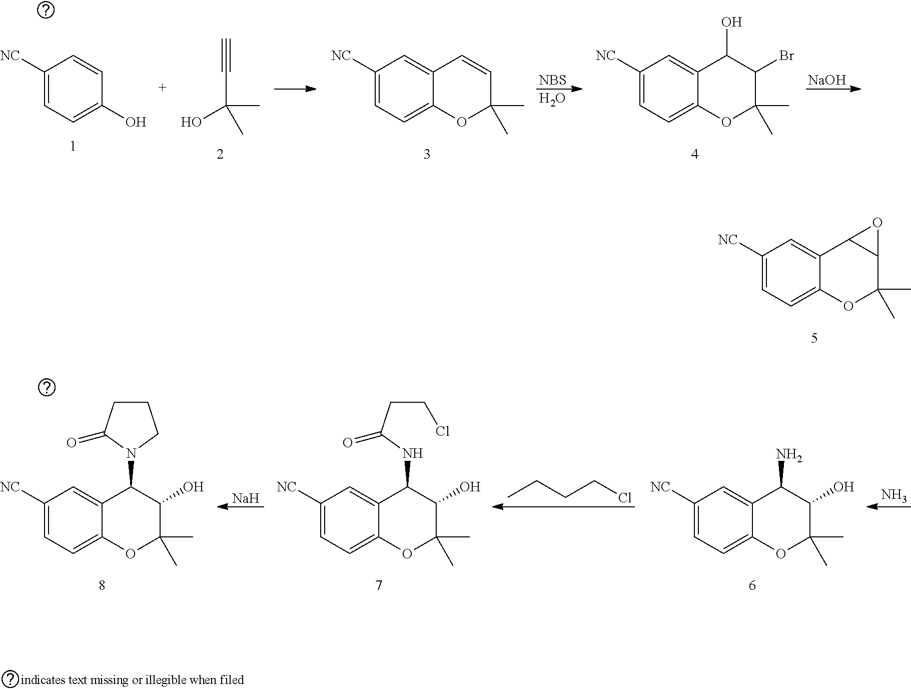

Reaction of 4-cyanophenol (4-Hydroxybenzonitrile) with 2-hydroxy-2-methyl-3-butyne under phase transfer catalyst probably proceeds to initial formation of a propargyl carbocation. The course of the reaction can be envisaged by assuming that this then attacks the aromatic ring; the resulting allylic cation can then capture the adjacent phenol oxygen and thus form the observed product (3). Treatment of that product with aqueous N-bromosuccinimide leads to the addition of the elements of hypobromous acid and formation of the bromohydrin (4) as a mixture of the trans enantiomers. This cyclizes to the epoxide 5 in the presence of sodium hydroxide (5). Ring opening of the oxirane with ammonia gives a mixture of the trans amino alcohols (6). These are probably resolved at this stage and the 3S,4R-enantiomer used in the next stage. That isomer is next acylated with 4-chrdorobutyl chloride to give the chloroamide (7). The anion from reaction of the amide with sodium hydride then displaces the chlorine on the end of the chain to form the pyrrolidine ring. There is thus obtained levcromakalim (8).

### Pharmaceutically Acceptable Salts

Salts of the compounds of Formula I, II or III described herein may be prepared by a range of known methods. These include, but are not limited to, reacting the compound with an alkali metal hydroxide or alkali metal alkoxide, such as for example, NaOH, KOH or NaOCH₃, in a variety of solvents which may be selected for example from low molecular weight ketones (e.g., acetone, methyl ethyl ketone, and the like), tetrahydrofuran (THF), dimethylformamide (DMF), and n-methylpyrrolidinone, and the like. In one embodiment the solvent is water. In another embodiment the solvent is THF.

The compounds described herein, may also form salts with organic cations that include at least one tertiary amine or ammonium cation. Organic cation compounds can have +1, +2, +3, or +4 charge per molecule by inclusion of one, two, three or four tertiary amine or ammonium ions within the compound, respectively. When a multicharged compound is used, the tertiary amine or quaternary ammonium moieties are preferably separated by a chain of at least 4 atoms, more preferably by a chain of at least 6 atoms, such as for example, hexamethyl hexamethylene diammonium dihydroxide, wherein the quaternary ammonium moieties are separated by —(CH₂)₆—.

Salts of the compounds described herein, may be prepared by reacting the compound with compounds that include at least one tertiary amine or quaternary ammonium ion (e.g., choline hydroxide, hexamethylhexamethylene diammonium dihydroxide) in a solvent selected from low molecular weight ketones (e.g., acetone, methyl ethyl ketone), tetrahydrofuran, dimethylformamide, and n-methyl pyrrolidinone. As with the preparation of salts from alkali metal hydroxides, amine and ammonium containing compounds typically do not form salts when the solvent is an alcohol.

Typically, basic addition of salts of the compounds described herein, may include those containing hexamethyl hexamethylene diammonium, choline, sodium, potassium, methyldiethyl amine, triethylamine, diethylamino-ethanol, hydroxyethyl pyrrolidine, tetrapropylammonium and tetrabutylphosphonium ions.

Typically, basic addition of salts of the compounds described herein, may be prepared using any suitable reagent, for example, hexamethyl hexamethylene diammonium dihydroxide, choline hydroxide, sodium hydroxide, sodium methoxide, potassium hydroxide, potassium methoxide, ammonium hydroxide, tetrapropylammonium hydroxide, or tetrabutylphosphonium hydroxide. The basic addition of salts can be separated into inorganic salts (e.g., sodium, potassium and the like) and organic salts (e.g., choline, hexamethyl hexamethylene diammonium hydroxide, and the like).

Salts of the compounds described herein may include organic or inorganic counter ions, including but not limited to, calcium, dimeglumine, dipotassium, disodium, meglumine, polistirex, or tromethamine. Suitable, organic cations include compounds having tertiary amines or quaternary ammonium groups.

Pharmaceutically acceptable salts of the compounds described herein may also include basic addition of salts such as those containing chloroprocaine, procaine, aluminum, calcium, lithium, magnesium, potassium, sodium, ammonium, and alkylamine. For example, see Remington's Pharmaceutical Sciences, 19th ed., Mack Publishing Co., Easton, Pa., Vol. 2, p. 1457, 1995.

Salts of the compounds described herein, may be prepared, for example, by dissolving the free-base form of a compound in a suitable solvent, such as an aqueous or aqueous-alcohol in solution containing the appropriate acid and then isolated by evaporating the solution. In another example, a salt is prepared by reacting the free base and acid in an organic solvent.

Solvents useful in the preparation of pharmaceutically acceptable salts of the compounds described herein include organic solvents, such as for example, acetonitrile, acetone, alcohols (e.g., methanol, ethanol and isopropanol), tetrahydrofuran, methyl ethyl ketone (MEK), ethers (e.g., diethyl ether), benzene, toluene, xylenes, dimethylformamide (DMF), and N-methylpyrrolidinone (NMP), and the like. In one embodiment the solvents are selected from acetonitrile and MEK.

In certain embodiments, salts described herein can be formed via ion exchange chromatography. When using ion exchange chromatography, the resulting cation is the cation that was present in the ion exchange wash solution.

For example, the sodium or potassium salt (shown below) can be generated by using NaHCO₃ for K₂CO₃, KHCO₃ or KOH, to afford the salt.

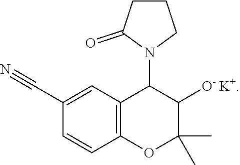

For example, the ammonium salt can be generated by using (NH₄)₂CO₃ or NH₄OH, to afford the salt.

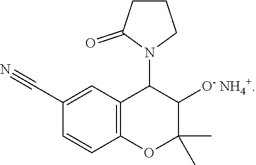

For example, the calcium salt can be generated by using CaCO₃ or Ca(OH)₂, to afford the salt.

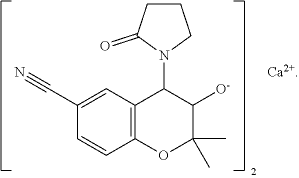

For example, the calcium salt can be generated by substituting 1M NaHCO₃ for 1M Li₂CO₃ or LiOH, to afford the salt.

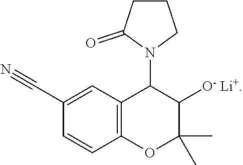

Other column material, salt washes, and concentrations can be used as desired.

### Synthetic Salt Formation

In certain embodiments, the phosphate esters described herein can be formed by direct chemical reaction as an alternative to ion exchange. For example, to generate a sodium salt of the compounds described herein, the acid version of the compound can be reacted with an aqueous solution or base solution such as NaOH, NaHCO₃, Na₂CO₃, or sodium acetate in a reaction vessel. In certain embodiments, other aqueous solutions may be used. For example, potassium hydride, lithium hydride, calcium hydride, acetate salts, sulfate salts, phosphate salts, and the like.

In certain embodiments, the chemical reaction can occur wherein the equivalence ratio is the same, for example a 1:1 ratio or wherein the equivalence ratio is different, for example, a ratio of 1:10; 1:5; 1:3; 1:2; or 1:1.5 CKLP1 to cation source. Concentrations of salt in solution can also be varied. For example, the chemical reaction can occur wherein the sample is washed with 1M aqueous NaHCO₃; however, the chemical reaction can also occur where the sample is washed with <1M or >1M aqueous NaHCO₃ as desired. This variance in equivalency is also applicable for chemical reactions involving other salts or base solutions as desired to afford a salt of the present invention.

Alternately, other salts may be prepared from the following: (a) metal hydroxides, for example any alkali metal hydroxides (e.g., NaOH and KOH), divalent metals (such as magnesium, calcium, and the like), and (b) organic hydroxides, for example organic compounds which include at least one tertiary amine, ammonium group, or at least one quaternary ammonium ion (e.g., diethylaminoethanol, triethylamine, hydroxyethylpyrrolidine, choline and hexamethylhexamethylenediammonium, and the like).

This specification has been described with reference to embodiments of the invention. However, one of ordinary skill in the art appreciates that various modifications and changes can be made without departing from the scope of the invention as set forth herein. Accordingly, the specification is to be regarded in an illustrative rather than a restrictive sense, and all such modifications are intended to be included within the scope of invention.

We claim:

1. An extended-release pharmaceutical composition comprising a biodegradable microparticle or nanoparticle formulation of cromakalim Formula I, 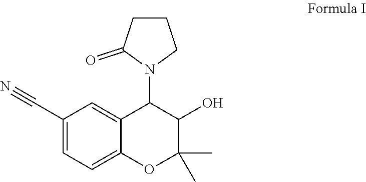 [SMILES: CC1(C)Oc2ccc(C#N)cc2C(N2CCCC2=O)C1O]
or a pharmaceutically acceptable salt thereof.

2. The extended-release pharmaceutical composition of claim 1, wherein cromakalim is substantially in the levo stereoconfiguration.

3. The extended-release pharmaceutical composition of claim 1, wherein cromakalim is substantially in the dextro stereoconfiguration.

4. The extended-release pharmaceutical composition of claim 1, wherein cromakalim is in a mixture of levo and dextro stereoconfiguration.

5. The extended release pharmaceutical composition of claim 4, wherein the mixture of levo and dextro cromakalim is racemic.

6. The extended-release pharmaceutical composition of claim 1, wherein the compound is formulated in biodegradable microparticles.

7. The extended-release pharmaceutical composition of claim 6, wherein the microparticle has an average diameter from about 1 μm to about 100 μm.

8. The extended-release pharmaceutical composition of claim 1, wherein the compound is formulated in biodegradable nanoparticles.

9. The extended-release pharmaceutical composition of claim 8, wherein the nanoparticle has a particle size from about 0.5 nm to about 100 nm.

10. The extended-release pharmaceutical composition of claim 1, wherein the microparticle or nanoparticle formulation is formed using solvent evaporation, solvent removal, spray drying, phase inversion, coacervation, or low temperature casting.

11. The extended-released pharmaceutical composition of claim 1 wherein Formula I is selected from Formula IA, Formula IB, or Formula IC: 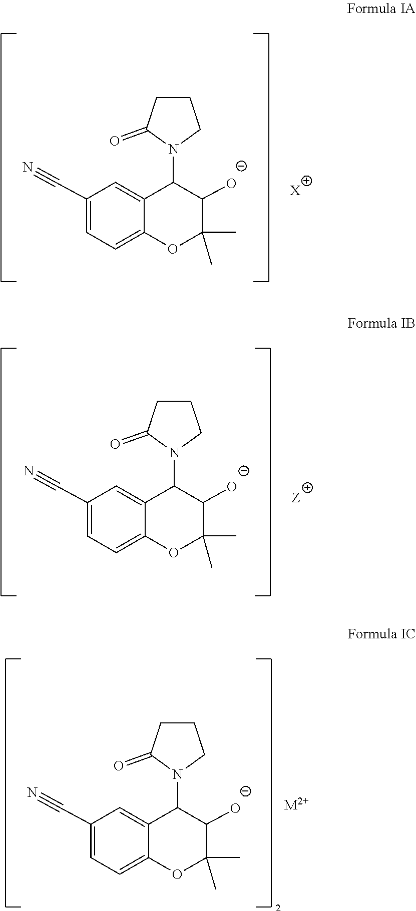
wherein X⁺ and M²⁺ are pharmaceutically acceptable cations and Z⁺ is a mixed salt cation of X⁺.

12. The extended-released pharmaceutical composition of claim 11, wherein the X⁺ cation is selected from sodium, potassium, aluminum, calcium, magnesium, lithium, iron, zinc, arginine, chloroprocaine, cesium, choline, diethanolamine, ethanolamine, lysine, histidine, meglumine, procaine, hydroxyethyl pyrrolidine, ammonium, tetrapropylammonium, tetrabutylphosphonium, methyldiethanamine, and triethylamine.

13. The extended-released pharmaceutical composition of claim 11, wherein the X⁺ cation is selected from an ammonium ion of the formula: 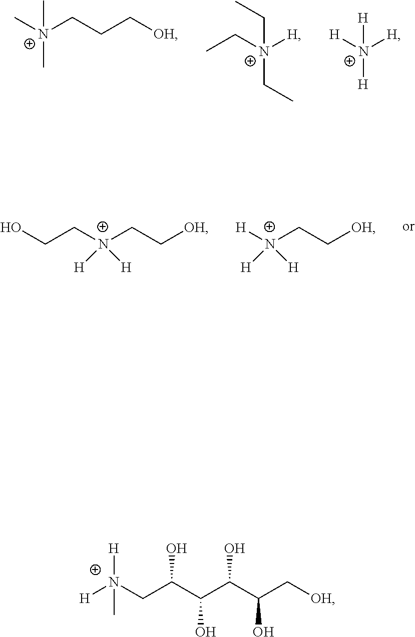
an ammonium ion of the formula: 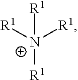
wherein R¹ is C₁-C₆ alkyl, aryl, wherein the C₁-C₆ alkyl or aryl.

14. The extended-released pharmaceutical composition of claim 11, wherein M² is an alkaline earth metal cation, a metal cation or an ammonium ion.

15. The extended-released pharmaceutical composition of claim 11, wherein the compound of Formula IA is selected from:

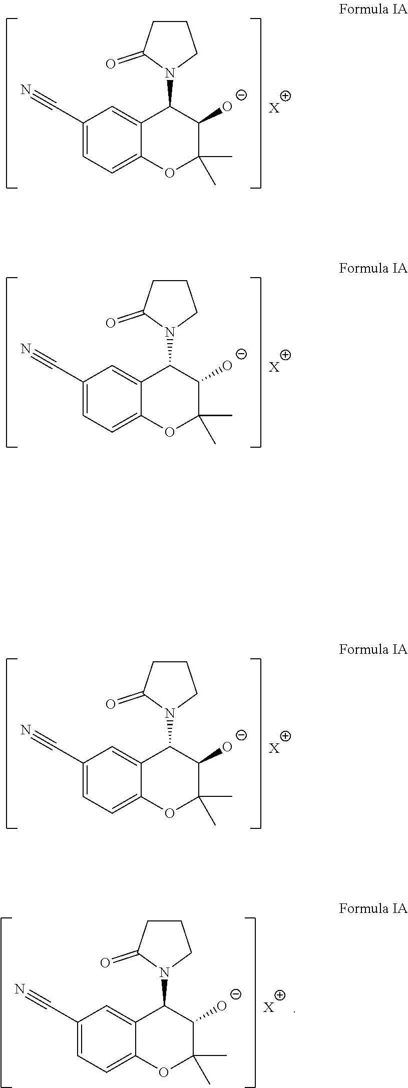

16. The extended-released pharmaceutical composition of claim 15, wherein the X⁺ cation is selected from sodium, potassium, aluminum, calcium, magnesium, lithium, iron, zinc, arginine, chloroprocaine, cesium, choline, diethanolamine, ethanolamine, lysine, histidine, meglumine, procaine, hydroxyethyl pyrrolidine, ammonium, tetrapropylammonium, tetrabutylphosphonium, methyldiethanamine, and triethylamine.

17. The extended-released pharmaceutical composition of claim 15, wherein the X⁺ cation is selected from an ammonium ion of the formula: 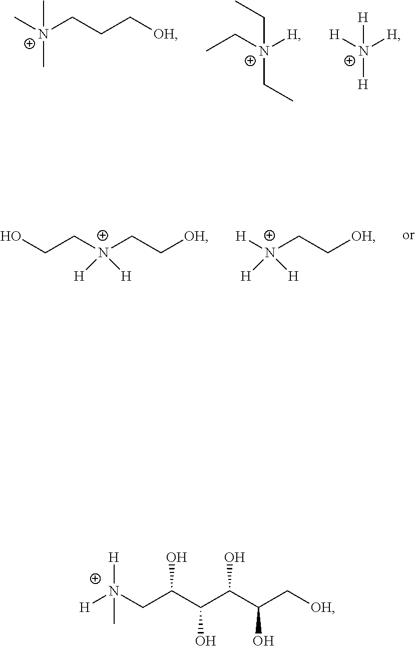
and
an ammonium ion of the formula: 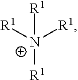
wherein R¹ is C₁-C₆ alkyl, aryl, wherein the C₁-C₆ alkyl or aryl.

18. The extended-released pharmaceutical composition of claim 11, wherein the compound of Formula B is selected from:

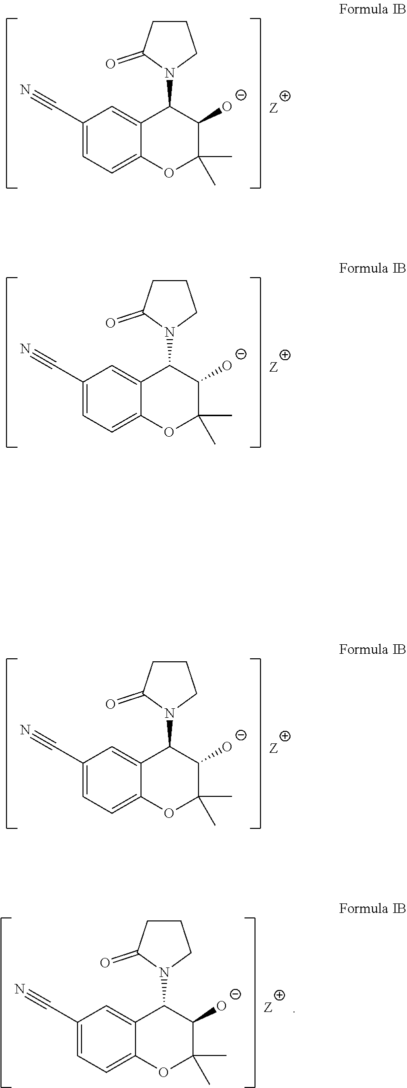

19. The extended-released pharmaceutical composition of claim 11, wherein the compound of Formula IC is selected from:

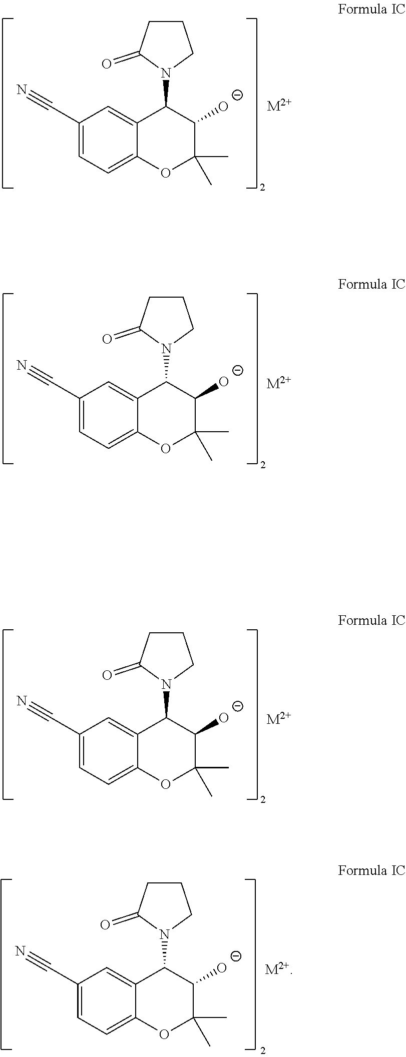

20. The extended-released pharmaceutical composition of claim 19, wherein M²⁺ is an alkaline earth metal cation, a metal cation or an ammonium ion.

21. A method of treating an ocular disorder, comprising administering an effective amount of an extended-release pharmaceutical composition of claim 1 to a host in need thereof.

22. The method of claim 21, wherein the ocular disorder is Sturge-Weber Syndrome.

23. The method of claim 21, wherein the ocular disorder is non-arteritic anterior ischemic optic neuropathy.

24. The method of claim 21, wherein the ocular disorder is Graves' opthalmopathy.

25. The method of claim 21, wherein the ocular disorder is cavernous sinus thrombosis, orbital vein vasculitis, or carotid-cavernous sinus fistula.

26. The method of claim 21, wherein the ocular disorder is orbital varices, central retinal vein occlusion, or branch retinal vein occlusion.

27. The method of claim 21, wherein the effective amount of an extended-release formulation does not cause significant hyperemia.
